SEQUENCE LISTING (57 sequences)

SEQ ID NO: 1 - DNA, 1395 bp, synthetic construct
  misc_feature 1..1395: note = psiD_PCU gene cassette
  source 1..1395: mol_type = other DNA
gctagcactagactcgagacaataatttcaaaccccgtaaggtattaaatgcatcaagttattccggcgtgcaattctgctgcaatccgctccctgtgcccgactccggaatctttccgtaacatgggttggctgtccgtgtctgacgcggtttactctgagtttatcggtgaactggcgactcgtgcttctaaccgcaattattctaacgaatttggcctgatgcagccaatccaagaatttaaagcgtttattgaatccgatccggttgtacatcaggagttcatcgatatgtttgaaggtatccaggatagcccgcgcaactaccaggagctgtgcaatatgtttaacgacatttttcgtaaagcacctgtttatggcgatctgggtccgccggtttacatgatcatggctaaactgatgaacacccgtgctggcttctccgctttcacccgtcagcgtctgaacctgcatttcaaaaagctgttcgatacgtggggtctgttcctgtctagcaaagattctcgtaacgtactggttgcggaccagttcgatgaccgtcattgtggctggctgaacgaacgtgctctgtccgcaatggttaagcactacaatggtcgtgcttttgatgaggtatttctgtgcgacaagaacgccccgtactacggttttaactcttacgacgatttcttcaaccgtcgttttcgcaaccgtgatatcgatcgcccggttgtaggtggtgtgaacaacactaccctgattagcgccgcttgtgagtctctgagctataacgtttcctacgacgtgcagtctctggataccctggtttttaaaggtgaaacttactccctgaaacatctgctgaacaacgatccgtttactccgcagtttgagcatggttccatcctgcagggcttcctgaacgttactgcttatcaccgttggcacgcaccggttaacggtactatcgttaaaatcatcaacgttccgggcacctacttcgcacaggctccgtctacgattggcgacccgatcccggataacgactacgaccctccaccgtatctgaaatctctggtttacttcagcaacattgcggcacgccagattatgtttatcgaagctgacaacaaggaaattggtctgatctttctggttttcattggcatgaccgaaatctctacgtgcgaagcaactgtgtctgaaggccagcacgttaaccgtggcgacgatctgggcatgtttcacttcggtggctctagcttcgctctgggcctgcgtaaggattgccgcgcagaaatcgtcgaaaaattcacggagccgggtaccgtcattcgtatcaatgaggttgttgcggctctgaaagcgcaccatcaccatcaccactaagtcgac

SEQ ID NO: 2 - DNA, 1565 bp, synthetic construct
  misc_feature 1..1565: note = rumD gene cassette: Ruminococcus gnavus TrpD^RG gene construct
  source 1..1565: mol_type = other DNA
gctagcactagactcgagcagggtcagtcgtcccttattttaacagttctaaggagggtatttttatgcatagccaagttattaagaagaaacgcaatacctttatgattggcaccgaatatatcctgaacagcactcagctggaagaagcaatcaaatctttcgtgcatgacttctgtgcagaaaagcatgaaatccatgaccagccggtggtagtggaggccaaagagcatcaggaggacaaaattaagcagatcaaaattccagagaaaggccgtccggttaacgaagtggtatctgagatgatgaacgaggtttaccgttaccgtggtgatgctaaccacccgcgtttcttctccttcgttccgggtccggcatctagcgtgagctggctgggcgacattatgaccagcgcgtataacattcatgctggtggcagcaaactggcgcctatggttaactgcattgaacaggaagtgctgaagtggctggctaagcaggtgggttttaccgaaaacccaggcggcgtgtttgtgtccggcggctccatggcgaacatcaccgcgctgactgctgctcgtgacaacaaactgaccgatattaacctgcacctgggtactgcctacatctccgaccagacccactcctccgtcgctaaaggcctgcgtatcatcggtattaccgatagccgcatccgtcgtatcccaaccaacagccactttcagatggacaccactaaactggaggaagctattgagactgataaaaagagcggttatatcccgttcgtagtcatcggcaccgctggtaccaccaataccggttccatcgatccactgacggaaatcagcgcgctgtgcaagaaacacgacatgtggttccatattgacggcgcttacggtgcgtctgtcctgctgagcccgaagtacaagtccctgctgaccggcaccggtctggctgatagcatctcttgggatgcacacaaatggctgtttcagacctacggttgcgcaatggtgctggttaaagacatccgcaatctgttccactctttccatgttaacccggaatatctgaaggatctggaaaacgacatcgacaacgttaacacttgggacatcggtatggaactgacccgtccagctcgcggtctgaaactgtggctgactctgcaggttctgggttccgacctgatcggttctgcgattgaacacggctttcagctggcggtttgggctgaagaggcgctgaacccaaagaaagactgggaaattgtttctccggctcagatggctatgattaactttcgctacgctccgaaagacctgactaaagaagaacaggacatcctgaacgagaaaatcagccatcgtatcctggaaagcggttatgcggctatctttactactgtgctgaacggcaaaacggtgctgcgtatttgtgctatccaccctgaagccacgcaggaagacatgcagcacactattgatctgctggaccagtacggtcgcgaaatctacacggaaatgaaaaaggcgcaccaccatcatcaccactaagtcgac

SEQ ID NO: 3 - DNA, 1331 bp, synthetic construct
  misc_feature 1..1331: note = cloD gene cassette: Clostridium sporogenes Trp^CS gene construct
  source 1..1331: mol_type = other DNA
gctagcactagactcgagtctccatagcacgaaattaaggggtcttttaaatgcataaattctggcgtaagtacactcagcaagaaatggatgagaaaatcaccgaatccctggaaaagaccctgaactacgataacaccaaaaccatcggtattccgggcactaaactggacgatactgttttctatgatgatcatagcttcgtcaaacactctccgtatctgcgtaccttcatccagaacccgaatcatatcggctgtcacacctacgataaagcggatatcctgttcggcggtaccttcgatatcgagcgcgaactgatccagctgctggctattgacgtgctgaacggtaacgacgaagagttcgacggttacgttactcaaggcggtacggaggcgaacatccaggcgatgtgggtttaccgcaactatttcaagaaggaacgtaaagctaaacacgaagaaattgcgattatcaccagcgcggacacccactatagcgcctacaaaggttccgatctgctgaacatcgatatcatcaaagtgccggtagacttctactcccgcaaaatccaggaaaacaccctggattccatcgtcaaggaagcaaaagaaatcggcaaaaagtacttcattgtcatttccaacatgggtacgaccatgtttggcagcgtggacgacccggatctgtacgcaaacatctttgacaaatataacctggagtacaaaatccacgtcgatggtgcttttggtggtttcatctacccaattgacaacaaagagtgcaaaaccgatttctctaacaaaaacgtctcctctatcacgctggacggccacaaaatgctgcaagctccgtacggtaccggtatttttgtgtctcgcaaaaacctgatccataacaccctgaccaaagaagctacctacatcgaaaatctggatgttaccctgtctggttcccgttctggtagcaacgccgtagcgatctggatggtgctggcgtcctacggcccgtatggctggatggagaaaatcaataaactgcgcaaccgtaccaaatggctgtgcaaacaactgaatgacatgcgtatcaaatactacaaggaggactccatgaacatcgtaaccattgaagagcagtatgtcaacaaagaaatcgctgagaagtacttcctggtgccagaagttcataacccgacgaataactggtacaaaattgttgtgatggaacacgtggaactggatatcctgaactctctggtgtacgatctgcgtaagttcaacaaagaacacctgaaagctatgcaccatcatcatcaccattaagtcgac

SEQ ID NO: 4 - DNA, 1166 bp, synthetic construct
  misc_feature 1..1166: note = PsiK_PCY gene cassette based on kinase from Psilocybe cyanescens
  source 1..1166: mol_type = other DNA
ctagcactagactcgagtttgacaagccaaccaaaaaccgaaggaatttttatatgcatacgttcgatctgaaaactgaagagggtctgctgagctatctgactaaacatctgtctctggatgttgcgccgaacggtgtcaaacgtctgagcggcggtttcgttaacgtcacttggcgtgttggtctgaacgcaccgtaccacggccatactagcatcattctgaaacatgcccagccgcatctgtcctccgatatcgacttcaaaatcggtgtggaacgttctgcctacgaataccaggcgctgaaaatcgttagcgcgaacagctctctgctgggttccagcgacatccgcgtgagcgtgccggaaggtctgcactacgatgtggttaacaacgcactgatcatgcaggatgtaggcacgatgaaaaccctgctggattatgttactgcgaaacctccgatctctgcggagatcgcgagcctggttggctcccaaattggtgctttcatcgcccgtctgcacaacctgggtcgtgaaaacaaagacaaagacgatttcaaattcttcagcggcaacatcgttggccgtaccaccgctgaccagctgtaccaaactatcatcccgaacgcagcaaagtatggcatcgacgacccgatcctgccgattgtggttaaagaactggttgaagaagttatgaacagcgaagaaaccctgatcatggcggatctgtggtctggcaacatcctgctgcagttcgacgagaacagcacggaactgactcgcatctggctggtagactgggaactgtgcaaatacggcccgccgtctctggatatgggctactttctgggcgactgtttcctggtcgcgcgcttccaggaccagctggtaggcaccagcatgcgtcaggcttacctgaaatcttatgctcgtaacgttaaggaaccgatcaactacgcaaaagccactgccggtatcggcgcgcacctggtcatgtggaccgatttcatgaagtggggcaacgacgaggagcgtgaagagttcgtaaagaaaggcgttgaagcgttccatgaagctaacgaagataaccgcaacggcgagatcacttccatcctggttaaagaagcatcccgtacccaccatcatcaccaccactaagtcgac

SEQ ID NO: 5 - DNA, 1168 bp, synthetic construct
  misc_feature 1..1168: note = PsiK_PCU gene cassette based on kinase from Psilocybe cubensis
  source 1..1168: mol_type = other DNA
gctagcactagactcgagctccagaagattaactagatacggacggttttagatgcatgctttcgacctgaagactgaagatggcctgattacttacctgaccaaacacctgagcctggacgtagatacctctggcgtaaaacgtctgtctggcggtttcgtgaacgttacctggcgtatcaaactgaacgcgccgtaccagggtcatacctctatcatcctgaaacacgcgcagccgcacatgtccaccgacgaagatttcaaaattggcgtcgaacgttctgtgtacgaataccaggctattaaactgatgatggcgaaccgcgaagtactgggtggtgttgacggcatcgtatccgtaccagaaggcctgaactatgacctggagaacaacgcactgatcatgcaagatgtgggtaaaatgaaaaccctgctggattatgttacggcgaaaccgccactggcaaccgatatcgcgcgtctggtcggtactgaaattggtggtttcgtagcgcgcctgcacaacatcggtcgtgaacgtcgtgatgatccggagtttaaattcttctccggtaacatcgtgggtcgtaccacctctgaccaactgtaccagaccatcattccgaacgcggcgaaatacggtgtggacgatccgctgctgccgactgttgtgaaagatctggttgacgacgtcatgcacagcgaagaaaccctggttatggccgatctgtggtctggtaacatcctgctgcaactggaagagggtaacccgtccaagctgcagaagatttacattctggactgggaactgtgtaaatacggtccggcatctctggatctgggctacttcctgggcgattgctatctgatctctcgtttccaggacgaacaggttggtactactatgcgtcaggcttacctgcagagctacgcgcgtacgtccaaacattctatcaactatgctaaagtaaccgcaggcatcgcggcgcacatcgttatgtggaccgacttcatgcaatggggctctgaagaggaacgtatcaactttgttaaaaagggtgtggcagcgttccacgacgctcgtggtaacaacgataacggcgaaatcactagcaccctgctgaaggaaagcagcaccgctcaccatcaccaccaccattaagtcgac

SEQ ID NO: 6 - DNA, 1002 bp, synthetic construct
  misc_feature 1..1002: note = PsiM-PCY gene cassette based on 4-hydroxytriptamine methylasefrom P. cyanescens
  source 1..1002: mol_type = other DNA
gaattcgctagcactagactcgagtactaaggtataaggaatctcaaaatgcatattcgtaatccgtatcgtgatggcgtagactaccaagctctggcagaagcattcccggctctgaaaccacatgtgaccgtaaactccgataacaccacttctatcgacttcgcggttcctgaagcacagcgcctgtacaccgcagctctgctgcaccgcgatttcggcctgactatcactctgccggaggatcgtctgtgcccgactgttccgaaccgtctgaactacgttctgtgggtggaagacattctgaaagttacttctgatgctctgggcctgcctgataaccgtcaggtcaaaggcattgatatcggtaccggtgcgtccgctatttacccgatgctggcttgctctcgttttaaaacttggagcatggttgcgacggaagttgaccaaaaatgtattgacaccgctcgcctgaacgtgatcgcgaacaacctgcaggaacgtctggcaatcattgcaaccagcgttgacggtcctattctggtgccgctgctgcaggcgaactctgatttcgagtacgacttcaccatgtgcaaccctccgttttacgacggtgcgtctgacatgcagactagcgatgcagctaaaggtttcggctttggcgttaacgcaccgcacaccggcaccgtgctggaaatggcaactgaaggcggcgagtctgcgttcgtagcgcagatggtgcgtgaaagcctgaacctgcagactcgttgccgttggttcacttctaacctgggtaagctgaaatccctgtacgaaatcgttggtctgctgcgtgaacatcagatttctaactatgcgatcaacgaatacgtacagggtgcaacccgtcgctatgcaatcgcgtggagcttcatcgacgtgcgtctgccggatcacctgtcccgtccgtctaacccggatctgagctccctgttccaccatcaccatcatcactaagtcgac

SEQ ID NO: 7 - DNA, 1014 bp, synthetic construct
  misc_feature 1..1014: note = PsiM_PCU gene cassette based on 4-hydroxytriptamine methylasefrom P. cubensis
  source 1..1014: mol_type = other DNA
gaattcgctagcactagactcgagcgcgccattccactttctctcttaaggcccctttttatgcatatccgcaatccttaccgtactccgatcgattatcaagcactgagcgaagccttcccgccgctgaaaccgttcgtttctgtgaacgcagacggcacttcttccgtcgatctgactattccagaagcacagcgcgccttcacggcggcgctgctgcatcgcgacttcggtctgaccatgactatcccggaggatcgcctgtgtccgaccgttccgaaccgtctgaactatgtgctgtggatcgaagacatcttcaactacaccaacaagactctgggtctgagcgacgatcgtccgatcaaaggtgtggacattggtaccggcgcctccgctatctacccaatgctggcgtgtgcgcgtttcaaggcatggtctatggttggcactgaggtggaacgcaaatgtattgacactgctcgcctgaacgtggtagcaaacaacctgcaggaccgtctgagcatcctggaaacctccatcgatggtcctattctggttccgatctttgaagcgaccgaagaatacgaatatgaatttactatgtgtaacccgccgttctacgacggcgcagccgatatgcagacgtctgacgcggctaagggtttcggcttcggcgtaggtgccccgcactctggcactgtgatcgagatgtccaccgaaggcggtgagtccgcctttgttgcccagatggtgcgtgaatctctgaaactgcgcacccgttgccgttggtatacctctaacctgggcaaactgaaatctctgaaagaaatcgtgggcctgctgaaagaactggaaatctccaactatgcgattaacgaatacgttcagggttctactcgtcgttacgcggttgcatggagcttcaccgatatccagctgccggaagaactgtcccgcccgtccaacccggaactgtcctctctgttccaccaccatcaccaccattaagtcgac

SEQ ID NO: 8 - DNA, 867 bp, synthetic construct
  misc_feature 1..867: note = rINMT gene cassette based on rabbit lung INMT enzyme
  source 1..867: mol_type = other DNA
gctagcactagactcgagtttgggctttcgagtaggaggttttagatgcataaagagggcggctttactggtggtgacgaatatcaaaaacatttcctgccgcgtgactacctgaacacctactactctttccaaagcggcccgtctccggaagcagagatgctgaaatttaacctggaatgcctgcacaaaacctttggcccgggtggcctgcagggtgacactctgatcgacattggctctggcccgactatctatcaggtactggccgcctgtgaatccttcaaagacatcaccctgagcgacttcaccgatcgtaaccgtgaagagctggccaagtggctgaagaaagagccgggtgcctatgattggactccggcactgaaatttgcctgcgaactggaaggcaacagcggtcgttggcaggaaaaagccgaaaaactgcgcgcaaccgtaaaacgcgttctgaaatgtgacgccaatctgtctaacccgctgaccccagttgttctgccaccagccgactgtgtcctgaccctgctggctatggaatgtgcttgttgttccctggatgcgtaccgcgctgccctgcgcaacctggcttctctgctgaaaccgggtggtcacctggtgaccaccgttaccctgcagctgtcttcttacatggttggcgaacgtgaattttcctgcgtcgctctggagaaagaagaagtggagcaggccgtgctggatgcaggctttgatattgaacagctgctgtacagcccgcaatcctacagcgcttctaccgccccgaatcgcggcgtttgctttctggttgcgcgtaagaagccgggctcccatcaccatcaccaccactaagtcgac

SEQ ID NO: 9 - DNA, 849 bp, synthetic construct
  misc_feature 1..849: note = rINMT-h gene cassette: rINMT gene construct with his tag removed
  source 1..849: mol_type = other DNA
gctagcactagactcgagtttgggctttcgagtaggaggttttagatgcataaagagggcggctttactggtggtgacgaatatcaaaaacatttcctgccgcgtgactacctgaacacctactactctttccaaagcggcccgtctccggaagcagagatgctgaaatttaacctggaatgcctgcacaaaacctttggcccgggtggcctgcagggtgacactctgatcgacattggctctggcccgactatctatcaggtactggccgcctgtgaatccttcaaagacatcaccctgagcgacttcaccgatcgtaaccgtgaagagctggccaagtggctgaagaaagagccgggtgcctatgattggactccggcactgaaatttgcctgcgaactggaaggcaacagcggtcgttggcaggaaaaagccgaaaaactgcgcgcaaccgtaaaacgcgttctgaaatgtgacgccaatctgtctaacccgctgaccccagttgttctgccaccagccgactgtgtcctgaccctgctggctatggaatgtgcttgttgttccctggatgcgtaccgcgctgccctgcgcaacctggcttctctgctgaaaccgggtggtcacctggtgaccaccgttaccctgcagctgtcttcttacatggttggcgaacgtgaattttcctgcgtcgctctggagaaagaagaagtggagcaggccgtgctggatgcaggctttgatattgaacagctgctgtacagcccgcaatcctacagcgcttctaccgccccgaatcgcggcgtttgctttctggttgcgcgtaagaagccgggctcctaagtcgac

SEQ ID NO: 10 - DNA, 866 bp, synthetic construct
  misc_feature 1..866: note = sINMT gene cassette based on Spider (Araneus ventricosus) INMT
  source 1..866: mol_type = other DNA
gctagcactagactcgagtgcacaccgaaatcagtaaggatatacaaatggaaaacgacgaagtgaaaatcaaggaagactacctgaaaaatctggaccctaagaaattccaggaatttaaactggaaggtctgaaggcagaaaaatctgaaaacaactacgatttttggttcaaatgcctgcatgagttctttagcagcggtaactgttccggtacccgcatgctggaaattggttccggtccgaccgtacataacatcgccaccgcatctgcctatttcccgtacatcgtgcaatccgaatttgtcgaatctaattgtgaacagctgcgcaagtggctgcgcaaggacccggattctctggattggaccattttcctgcaacgtgtagcacgtgcagaaggtaaggaagatgttgacgccgcggtcccagagatcgaagatcgtatccgttctgcggttaaagcagtggtccatgcagatgttctggacgaccaagtcatcccgaaagaatatacgcaggaaccgtatgatctgattctgagcgtactgaccttcgaaaccgcggctgttaatctggaatcctattccaagattctgggccgtgctaatcgtctgctgcgcaagggcggtaaaatgattctgatttctgcaactaaatgtacctactggcaggttggcggtaacaaattccactgcctgtccctggaagaacacgaaattgaagatgcactgaaaaagaatggttttggcgagatctgctggaaaatccgtgaacgtccggacgatgatctgccgttcaactgcaagaacgtatactttctgacctgtacgaaactgcaccaccatcaccaccactaagtcgac

SEQ ID NO: 11 - DNA, 750 bp, synthetic construct
  misc_feature 1..750: note = wINMT gene cassette based on Roundworm (Anyclostoma ceylanicum)INMT
  source 1..750: mol_type = other DNA
gctagcactagactcgagctacactttatttcctaaggatccgtttttatgtctctgttcgcactgccagtttttgcacacactattcagaagaccatgccggcggaagaacgccagaccctgctggacattggcgcaggcccgacggtgtattccgccctgtgctttcgcgaggtggtaaaacgtgtctacctgtccgactacctgtccaaaaacctggacatcctgaaagcctggcgttccaacaccagcgcttacgattggaaaccgactattaaagtcatcctgcgtaccgagggcatgctgccagcaagcgattccgaaatggaagaagtggaagaacgtgcgcgtgcagcgctgaagtgcggcggtatcctgtacgccaacgtacatgaagacccggtagttccggatatgcgcggcgaacaggttgatgtactggtgaccattttcaccctggaaagcgcgtgtaaatcttacaccgagtattgtcagtgcgtaaaaaacatgctgaagcacctgcgcccaggtggccgcctggttctgggtagcgtgctggaagaggaatcttacaactccggtaaagacgtgatcttccacctgctgcatctgtccgaagatcagattctgggcgcactgggctctgctggtattgatctgaatagcgtcaagaaatacgttctggatgaagatggcgtaatgttcctgatggctgttaagaaccaccatcaccatcatcactaagtcgac

SEQ ID NO: 12 - DNA, 865 bp, synthetic construct
  misc_feature 1..865: note = fINMT gene cassette based on American bullfrog (Lithobatescatesbeianus) INMT
  source 1..865: mol_type = other DNA
gctagcactagactcgaggtcactactcacgaaacttaaacttaggccaggttattttatggactctactacccacaaactgtatcacgttcacggtatggattcccgtggcttcctggatacgtacttttctaacaaagaggacatggttttcgccgaagactccatcaaatttccaatggccatgttccactatcagttctccagcggccgcatcgagggcactttcctgatcgacatctctctgggttccgttattcacaacctgtactctgcttccaacagcttcaaaaacatcgttattctgaaatttcaagaaaaatgtatcatggaactgaaccgttggctgcatgatcgtaccggtgcttatgactggagccacacctcctctgcggctgcggaactggagggcacccgtgatcagctgcaagaaaaggaaatgtgcctgcgtagcagcatccgccagatcctgaaatgcgacttcgagcaggaaaacaccattagcccgatgctgctgccgctggctgactgcattatcagcgcgtggattctggaggtaatcagcaaaaacgaagatgaatacatgaaaaatctggagaaaatcatcaaactgctgaaaccgggtggccagctgctgctgattgcatccctggatgcaacctattggtttgctggtgcaggtcgctttcactgcttcaaaattaacgaagaatttgtcaaaaacgcgtttggcaagctgggtctggtgatcgactattacgccgtgcagcgtcgtcgtaacgttagcgacattatcgactataaggccatgctgttcatcgttggccgcaaaggcgagcatcaccaccatcaccattaagtcgac

SEQ ID NO: 13 - DNA, 775 bp, synthetic construct
  misc_feature 1..775: note = E. coli mtnN gene cassette
  source 1..775: mol_type = other DNA
gctagcactagactcgagaaagaatttgatttaagtaggagtaaggagtatgcataagattggtatcattggcgcgatggaagaggaagtaaccctgctgcgcgacaagatcgaaaaccgtcagactatttctctgggtggttgcgaaatctacaccggccaactgaacggcacggaagttgctctgctgaaatccggtattggtaaagtcgcggcagcactgggtgcgactctgctgctggaacactgcaaaccggacgtgattatcaacaccggcagcgcaggtggcctggcaccgaccctgaaagtgggcgacatcgtggtgtctgacgaagcgcgttatcacgatgctgacgtgaccgctttcggttatgagtatggtcaactgcctggctgcccggcaggtttcaaagcagacgataaactgattgcggcggctgaagcctgtatcgcagaactgaacctgaacgctgtgcgcggcctgattgtttctggtgacgcttttattaatggtagcgttggcctggccaaaattcgtcacaacttcccgcaggcaatcgctgtagaaatggaagctaccgcaattgcgcacgtatgtcacaacttcaacgttccgtttgtagtggtgcgcgccatctccgatgtcgctgatcagcagtctcacctgagcttcgacgaatttctggcagtagctgctaaacagagctccctgatggttgagtctctggtacagaaactggcccacggccaccaccatcaccaccactaagtcgac

SEQ ID NO: 14 - DNA, 1594 bp, synthetic construct
  misc_feature 1..1594: note = psiH_PCY gene cassette: araP_mRNA_psiH_PSICY_50820.23=>4314.79.Biobasic code: H-PSICY2. Cloning restriction sites: EcoRI, SalI;Vector: pUC57-Km
  source 1..1594: mol_type = other DNA
gctagcactagactcgagggatatttctcaaaggaccttttttatgcatattgtactgctggtatctctggttctggctggctgtatttactacgcaaacgcacgccgtgtgcgtcgcagccgcctgccaccgggccctccgggcatcccgctgccgtttattggcaacatgttcgatatgccttccgagtctccgtggctgcgtttcctgcagtggggtcgtgactaccacactgacatcctgtacctgaacgcaggtggtaccgaaatcattatcctgaacacgctggatgcaatcaccgatctgctggaaaagcgtggctctatgtattccggtcgtctggaatccactatggttaacgagctgatgggctgggagttcgacctgggtttcattacctatggtgaacgttggcgcgaagaacgtcgtatgttcgctaaagagttcagcgaaaagaacattcgtcagttccgtcatgcgcagattaaagcggcaaaccagctggtgcgtcagctgatcaaaactccggaccgttggtcccagcatattcgtcaccagattgccgcaatgtctctggatatcggttatggcatcgacctggcagaagatgatccgtggatcgcggcaacccagctggctaacgaaggcctggcggaagcctctgtaccgggcagcttctgggtcgatagcttcccggctctgaaatatctgccgtcctggctgccgggtgcgggtttcaaacgcaaagcaaaagtgtggaaagaaggcgcagaccacatggtaaatatgccatacgaaactatgaagaaactgactgttcagggtctggctcgcccaagctacgcaagcgctcgtctgcaagcgatggacccagatggcgatctggaacaccaggaacacgttattcgtaacacggctaccgaggttaatgtgggtggtggtgatactaccgtttctgccgtgtccgccttcattctggcaatggtcaaatacccggaagtgcagcgtcaggttcaggctgaactggacgctctgaccagcaaaggtgttgtacctaactatgacgaagaagacgacagcctgccgtacctgaccgcctgtgttaaagagatttttcgctggaatcagattgcaccgctggctatcccgcaccgtctgatcaaagacgacgtttatcgcggttatctgatcccgaaaaacgccctggtgtacgctaacagctgggcggttctgaatgatccggaagaatacccgaacccgagcgagtttcgtccggaacgttacctgtcttctgacggtaaaccggacccaactgtgcgtgacccgcgcaaagcggcgttcggttatggtcgtcgtaactgtccgggtatccacctggcacagtctaccgtttggattgctggcgcaactctgctgtctgtgtttaacatcgaacgcccggttgatggcaacggcaaaccgatcgacattccggctaccttcaccaccggtttcttccgtcatccggaaccgtttcaatgtcgtttcgtgccgcgcacccaagagatcctgaaatccgtgagcggtcatcaccatcatcaccactaagtcgac

SEQ ID NO: 15 - DNA, 2057 bp, synthetic construct
  misc_feature 1..2057: note = chuA gene cassette: encoding hemin receptor from Escherichia coliO157H7 str. EDL933; Biobasic code: CHUA; Cloning: EcoRI, SalI;Vector: pUC57-Km
  source 1..2057: mol_type = other DNA
gctagcactagactcgagatttctgaacatcaaaggagcatataatgcattctcgtccacaatttacttctctgcgtctgtctctgctggcactggctgtgtccgccactctgcctacgtttgcgttcgctactgaaaccatgactgttactgcgactggtaatgcgcgctcttccttcgaagccccgatgatggtttctgtaattgacacctctgctccggaaaaccagaccgctacttccgcgaccgacctgctgcgccacgttccgggcattaccctggatggcacgggtcgtactaacggtcaagatgtgaacatgcgtggttacgaccaccgcggcgtactggtgctggttgatggtgttcgccagggtacggatactggccacctgaacggtaccttcctggacccggctctgatcaagcgcgttgagatcgtacgtggcccgagcgcgctgctgtacggttctggcgcgctgggcggcgttatcagctacgatactgttgacgcgaaggatctgctgcaggaaggtcagtccagcggtttccgtgtattcggcacgggtggtaccggcgatcactccctgggcctgggtgcttctgctttcggccgcactgaaaacctggatggcatcgttgcatggtcttctcgcgatcgcggtgacctgcgtcagtccaatggcgaaactgcaccgaacgacgaatctattaacaacatgctggcaaaaggtacgtggcagatcgactccgcgcagagcctgtctggcctggtgcgctattataacaacgatgctcgtgaaccgaaaaacccgcagacggttggcgcgtccgaaagcagcaatccgatggttgaccgttccactatccagcgtgacgcgcagctgtcctacaaactggcaccgcaaggtaacgattggctgaacgctgatgccaagatttactggtccgaagtacgtatcaatgcacaaaacacgggctcttctggcgagtaccgcgaacaaattaccaaaggcgctcgtctggagaatcgctctaccctgttcgccgacagcttcgcttctcacctgctgacttacggtggtgaatattaccgtcaggaacagcaccctggtggcgctaccaccggcttccctcaggcaaagatcgacttcagcagcggttggctgcaggatgaaatcaccctgcgcgacctgccgatcaccctgctgggcggtacccgttatgattcttaccgtggttcctccgatggctacaaagacgttgacgcggataaatggtcttcccgtgctggtatgaccattaacccgacgaactggctgatgctgttcggttcctacgcgcaagccttccgtgctccgaccatgggcgaaatgtacaatgactctaaacacttctctatcggtcgcttctacaccaactattgggtgccgaacccgaatctgcgtccggagactaacgaaacccaagagtacggcttcggtctgcgcttcgatgacctgatgctgtccaacgacgccctggaatttaaagcctcttacttcgataccaaagcaaaagactatatctccaccactgtcgatttcgcggcagcaaccactatgtcttacaacgttccgaacgcgaaaatctggggttgggatgttatgaccaagtacaccactgacctgttctctctggacgtagcgtataatcgcactcgtggcaaagacaccgataccggcgaatacatcagctccattaacccggacaccgtaacttctaccctgaacatcccaatcgcgcactccggcttctctgttggttgggtgggcacgttcgcggatcgttccactcacatttcctcttcttacagcaaacagccgggttatggtgtgaacgacttctatgttagctaccagggtcagcaggctctgaagggcatgaccactactctggtcctgggtaacgcgttcgataaggaatactggagccctcagggtattccgcaggatggccgtaacggcaaaattttcgtgtcttatcaatggtggcaccatcatcaccaccattaagtcgac

SEQ ID NO: 16 - DNA, 1594 bp, synthetic construct; identical to SEQ ID NO: 14
  misc_feature 1..1594: note = rCPR gene cassette: araP mRNA rat P450 reductase NsiI-SalI47892.89=>5863.21=>5039.93. Biobasic code: rCPR; Cloningrestriction sites: EcoRI, SalI; Vector: pUC57-Km
  source 1..1594: mol_type = other DNA

SEQ ID NO: 17 - DNA, 2332 bp, synthetic construct
  misc_feature 1..2332: note = pCPR gene cassette; Psilocybe cyanescens uncharacterized geneCVT25_015047 with homology to NADPH cytochrome P450 reductase.araP_mRNA_pCPR; Biobasic code: pCPR; Cloning: EcoRI, SalI;Vector: pUC57-Km
  source 1..2332: mol_type = other DNA
gctagcactagactcgagagacctcacgccctaacccatcctccggacgttttttatgcataccgatcctaaccgtactactttctcttctgctctgcacccgctggcagttgtatctatggcgtccagctccagcgacgttttcgtgctgggcctgggcgttgttctggcggcgctgtatattttccgtgatcagctgtttgcagcatctaaaccaaaggtggccccggtttccaccaccaaaccggcgaacggcagcgcgaacccgcgtgacttcatcgctaaaatgaaacaaggtaagaaacgtatcgttatcttctacggcagccagaccggtactgctgaagagtatgctatccgtctggcgaaagaagcgaaacaaaaattcggtctggcgtctctggtttgtgatccggaggaatatgatttcgagaaactggatcagctgccggaagactccattgcgttcttcgtggttgcgacctacggcgaaggcgagcctacggataacgcggtgcaactgctgcagaacctgcaggacgactccttcgaattttctaacggtgaacgcaaactgtctggcctgaaatacgttgtattcggtctgggcaataagacttacgagcactataacctgatcggtcgtaccgtagacgctcagctggcgaaaatgggcgcagtgcgtgtgggtgaacgcggcgaaggtgacgatgacaaaagcatggaagaagattatctggagtggaaagatggtatgtgggacgcgttcgcagcagcaatgggtgtcgaagaaggtcagggtggtgacagcgctgatttcgttgtgtccgaactggaaagccacccgccagaaaaagtttacctgggtgaatactctgcgcgcgctctgactaaaaccaaaggtattcatgatgccaaaaatccgctggctgccccgatcactgtggcacgcgaactgtttcagagcgtagtggaccgcaactgtgttcacgtggagttcaacattgaaggttctggcattacgtaccaacacggcgatcacgttggtctgtggccgctgaacccggatgttgaagttgaacgtctgctgtgtgtgctgggtctgaccgagaaacgcgatgcggttattagcatcgaatctctggacccggcactggcaaaagttccgtttccggtgccgaccacctacgcggcagttctgcgtcactatattgatgtttctgcggtcgcaggccgtcagatcctgggtactctgtccaaattcgcgccgaccccggaagcggaagcgtttctgaaaaacctgaataccaacaaagaggaataccataatgtggttgcgaacggttgtctgaaactgggtgaaatcctgcaggttgctacgggtaacgatatcactgtagctccaacccctggtaacactaccaaatggccgattcctttcgacattatcgtaagcgccattccgcgtctgcagccgcgttattacagcatcagctcttcccctaaagttcacccgaacactatccatgcgaccgtggttgtcctgaaatacgagaacgtaccgaccgatcctatcccgcgtaagtgggtgtatggtgtaggtagcaatttcctgctgaacctgaaacatgcgattaacaaagagccggtcccgttcatcacgcagaatggtgaacagcgcgttggtgttccggaatatctgattgctggtccgcgtggcagctacaaaactgaatctcacttcaaagcgccgattcacgtacgtcgcagcacgttccgcctgccgactaaccctaaaagcccggttattatgattggtccgggtactggtgttgcaccgttccgtggtttcgtgcaggaacgtgttgcgctggctcgtcgttctgtggagaaaaacggtccggaatctctgaatgactggggccgcatctccctgttctacggctgccgccgtagcgacgaagatttcctgtacaaagacgaatggccgcagtaccaagaagaactgaaaggtaaattcaaactgcattgcgcattctctcgcgaaaactacaaaccggatggtagcaaaatctatgtgcaggacctgatctgggaggatcgcgagcatatcgcggacgccatcctgaatggcaagggttacgtatacatttgcggtgaggcgaaatctatgagcaaacaagtcgaggaggttctggcacgtatcctgggcgaggcgaaaggcggctccggtgctgttgaaggcgtggctgaaatcaaactgctgaaagagcgttcccgtctgatgctggattacgaactggcccaccaccatcaccaccattaagtcgac

SEQ ID NO: 18 - DNA, 2319 bp, synthetic construct
  misc_feature 1..2319: note = aCPR gene cassette: Agrocybe pediades uncharacterized gene withhomology to NADPH cytochrome P450 reductase. araP_mRNA_aCRP;Biobasic code: aCPR; Cloning: EcoRI, SalI; Vector: pUC57-Km
  source 1..2319: mol_type = other DNA
gctagcactagactcgagattcttgtcgtcagtattcaaaggataaccattatgcattcctattctaaccattctaccgcctttccaaatccgctgtctgcgtacaacatggcatccagcagctccgacgtcttcattctgggcctgggcatcgtactggcagcactgtacatcttccgtgatcagctgtttgcggccagcaaaccgaaagtggcgcctgtagccgcctccaaagcggcgaacggtagcggcaacccgcgtgatttcatcgccaaaatgaaagaaggcaagaaacgcatcgtgatcttctacggttcccagaccggcactgctgaagaatacgcgatccgtctggctaaagaggctaaacagaaatttggtctggcgtctctggtttgcgatccagaagaatacgattttgagaacctggatcaactgccggaagactgcgctgctttcttcgtgatggcaacgtacggcgaaggtgagccgactgataacgcagtacaactgatgcagaacctgcaggatgaatccttcgaattttctaacggtgaacgtaaactggaaggcctgaagtacgtggtattcggcctgggtaacaaaacctatgaacactacaacctgattggtcgtaatgtagatgctgccctgtccaaaatgggtgccatccgcatcggcgaacgtggtgaaggtgatgatgataaaagcatggaggaggactacctggaatggaaagacggcatgtgggaggcattcgcaaccgccatgaacgtggaggaaggtcagggcggtgatagcgcagacttcgtagtgtctgaactggaaagccaccctgaagataaagtgtacctgggtgaatactctgctcgcgccctgacgcgcaccaaaggtatccacgacgctaaaaacccgtatgctgcaccgatcgtcgttgcacgcgaactgttctctgctaacgccgatcgtaactgcgttcatatcgaatttaacacggaaggttctggtattacgtaccagcacggcgaccacgttggtctgtggccgctgaacccggatgtagaagtcgagcgtctgctgtgcgttctgggcctgacccagaaaaaggacaccgttatcggcatcgaaagcctggacccggcgctggctaaggtaccattcccggttcctactacctacggcaccgttctgcgccactacattgatatcagcgctgttgcgggccgtcagatcctgggcaccatgtccaaattcgctcctactcctgaagcggaagccttcctgaaaaacctgaacaccaacaaagaagaataccaccgtgttgtccacgacggttgcctgaaactgggtgaggttctgcaactggcagcgggtaacaacatcaaagcgattccgacttccgagaataccactcagtggaagatcccgttcgatattatcgtgtccgcaatccctcgtctgcagccgcgttactactccatttcttcttccccgaaactgcatccgaacagcattcatgcgacggctgttgtgctgaaatatcaaaatgttccgtccgagctggtaacttctaagtgggtgtacggtgtgggctccaacttcctgctgagcctgaaacatgcaagcaacaacgaaccgatcccagtgatctccgaaaacggcgaagagcgtgttgcagttccggcctatctgatcgaaggcccgcgtggcagctataaaaccgaaacccactacaaagcaccgatccacgtgcgtcgttctacctttcgtctgccgaccaacccgaagagcccggtaatcatggtgggtccgggcaccggcgtggcaccgtttcgtggcttcgtacaggaacgcgttgcactggcacgccgttccatcgaaaagaacggtccggatgcgctgaatgactggggccgcatctctctgttctatggttgccgtcgtgaggacgaggactttctgtataaagacgaatgggcaaaatacgaagaagaactgaaaggtaaatttaaaatgcactgtgcatttagccgtcagaactacaaaccggatggttctaagatctacgtacaggacctgatttgggaggaccgtgaacatatcgcggacgctatcctgaacggcaaaggttatgtctacatttgcggtgaggcaaaaagcatgtctaaacaggttgaagaggtactggctcgtatcctgggtgaagctaaaggcggctctgccgaagtcgaaggtgcggctgaaattaaactgctgaaagaacgctctcgtctgatgctggatgtttggagccaccatcaccaccatcactaagtcgac

SEQ ID NO: 19 - DNA, 1177 bp, synthetic construct
  misc_feature 1..1177: note = malE' gene cassette: araP_mRNA_malE'-6xhis_14755.50=>14746.21.Biobasic code: MALE. Cloning restriction sites: EcoRI, SalI;Vector: pUC57-Km
  source 1..1177: mol_type = other DNA
gctagcactagactcgagtttaatttataaaggagattagcatatgcataaaatcgaagaaggtaaactggttatctggatcaatggtgataaaggttataacggcctggcggaagtaggtaaaaagttcgaaaaagataccggcatcaaggtaaccgtggaacatccagacaaactggaagaaaagttcccgcaagttgctgctactggtgacggcccggatatcatcttctgggcgcacgaccgtttcggcggctacgcacaatctggcctgctggctgaaatcaccccggataaagcattccaggataaactgtacccgttcacctgggacgcggtgcgctacaatggcaaactgatcgcctacccgatcgccgttgaagcgctgtctctgatctacaacaaggacctgctgccgaacccgccgaagacctgggaagaaatccctgccctggacaaggaactgaaggctaagggtaaaagcgcgctgatgttcaacctgcaggaaccgtacttcacttggccactgattgctgctgatggcggttatgctttcaaatacgagaacggcaagtacgacattaaagacgtgggtgtggacaacgcaggcgcaaaagcaggtctgaccttcctggtggatctgatcaagaacaagcacatgaatgccgatactgactactccattgccgaggcggcttttaacaaaggcgaaactgctatgacgatcaacggtccttgggcttggagcaatatcgatacttccaaagttaactacggtgtaaccgttctgccaacgttcaagggccagccgtctaaaccgtttgtcggtgtgctgtctgctggtatcaacgcagcttctccgaacaaagaactggcaaaagaatttctggaaaattatctgctgaccgacgaaggtctggaagcagttaacaaagataaaccgctgggcgcggtagcgctgaaaagctacgaagaggagctggctaaggacccgcgtatcgccgcaaccatggagaacgcgcagaaaggcgaaattatgcctaacattccgcagatgtctgccttttggtacgcggtgcgtaccgccgttatcaacgccgcgtccggtcgtcagacggttgatgaagctctgaaagatgcacagactgtgcaccaccaccatcaccattaagtcgac

SEQ ID NO: 20 - DNA, 1320 bp, unidentified
  misc_feature 1..1320: note = psiD
  source 1..1320: mol_type = genomic DNA
atgcaagttattccggcgtgcaattctgctgcaatccgctccctgtgcccgactccggaatctttccgtaacatgggttggctgtccgtgtctgacgcggtttactctgagtttatcggtgaactggcgactcgtgcttctaaccgcaattattctaacgaatttggcctgatgcagccaatccaagaatttaaagcgtttattgaatccgatccggttgtacatcaggagttcatcgatatgtttgaaggtatccaggatagcccgcgcaactaccaggagctgtgcaatatgtttaacgacatttttcgtaaagcacctgtttatggcgatctgggtccgccggtttacatgatcatggctaaactgatgaacacccgtgctggcttctccgctttcacccgtcagcgtctgaacctgcatttcaaaaagctgttcgatacgtggggtctgttcctgtctagcaaagattctcgtaacgtactggttgcggaccagttcgatgaccgtcattgtggctggctgaacgaacgtgctctgtccgcaatggttaagcactacaatggtcgtgcttttgatgaggtatttctgtgcgacaagaacgccccgtactacggttttaactcttacgacgatttcttcaaccgtcgttttcgcaaccgtgatatcgatcgcccggttgtaggtggtgtgaacaacactaccctgattagcgccgcttgtgagtctctgagctataacgtttcctacgacgtgcagtctctggataccctggtttttaaaggtgaaacttactccctgaaacatctgctgaacaacgatccgtttactccgcagtttgagcatggttccatcctgcagggcttcctgaacgttactgcttatcaccgttggcacgcaccggttaacggtactatcgttaaaatcatcaacgttccgggcacctacttcgcacaggctccgtctacgattggcgacccgatcccggataacgactacgaccctccaccgtatctgaaatctctggtttacttcagcaacattgcggcacgccagattatgtttatcgaagctgacaacaaggaaattggtctgatctttctggttttcattggcatgaccgaaatctctacgtgcgaagcaactgtgtctgaaggccagcacgttaaccgtggcgacgatctgggcatgtttcacttcggtggctctagcttcgctctgggcctgcgtaaggattgccgcgcagaaatcgtcgaaaaattcacggagccgggtaccgtcattcgtatcaatgaggttgttgcggctctgaaagcgtaa

SEQ ID NO: 21 - DNA, 1473 bp, unidentified
  misc_feature 1..1473: note = rumD; Ruminococcus gnavus
  source 1..1473: mol_type = genomic DNA
atgagccaagttattaagaagaaacgcaatacctttatgattggcaccgaatatatcctgaacagcactcagctggaagaagcaatcaaatctttcgtgcatgacttctgtgcagaaaagcatgaaatccatgaccagccggtggtagtggaggccaaagagcatcaggaggacaaaattaagcagatcaaaattccagagaaaggccgtccggttaacgaagtggtatctgagatgatgaacgaggtttaccgttaccgtggtgatgctaaccacccgcgtttcttctccttcgttccgggtccggcatctagcgtgagctggctgggcgacattatgaccagcgcgtataacattcatgctggtggcagcaaactggcgcctatggttaactgcattgaacaggaagtgctgaagtggctggctaagcaggtgggttttaccgaaaacccaggcggcgtgtttgtgtccggcggctccatggcgaacatcaccgcgctgactgctgctcgtgacaacaaactgaccgatattaacctgcacctgggtactgcctacatctccgaccagacccactcctccgtcgctaaaggcctgcgtatcatcggtattaccgatagccgcatccgtcgtatcccaaccaacagccactttcagatggacaccactaaactggaggaagctattgagactgataaaaagagcggttatatcccgttcgtagtcatcggcaccgctggtaccaccaataccggttccatcgatccactgacggaaatcagcgcgctgtgcaagaaacacgacatgtggttccatattgacggcgcttacggtgcgtctgtcctgctgagcccgaagtacaagtccctgctgaccggcaccggtctggctgatagcatctcttgggatgcacacaaatggctgtttcagacctacggttgcgcaatggtgctggttaaagacatccgcaatctgttccactctttccatgttaacccggaatatctgaaggatctggaaaacgacatcgacaacgttaacacttgggacatcggtatggaactgacccgtccagctcgcggtctgaaactgtggctgactctgcaggttctgggttccgacctgatcggttctgcgattgaacacggctttcagctggcggtttgggctgaagaggcgctgaacccaaagaaagactgggaaattgtttctccggctcagatggctatgattaactttcgctacgctccgaaagacctgactaaagaagaacaggacatcctgaacgagaaaatcagccatcgtatcctggaaagcggttatgcggctatctttactactgtgctgaacggcaaaacggtgctgcgtatttgtgctatccaccctgaagccacgcaggaagacatgcagcacactattgatctgctggaccagtacggtcgcgaaatctacacggaaatgaaaaaggcgtaa

SEQ ID NO: 22 - DNA, 1254 bp, unidentified
  misc_feature 1..1254: note = cloD; Clostridium sporogenes
  source 1..1254: mol_type = genomic DNA
atgaaattctggcgtaagtacactcagcaagaaatggatgagaaaatcaccgaatccctggaaaagaccctgaactacgataacaccaaaaccatcggtattccgggcactaaactggacgatactgttttctatgatgatcatagcttcgtcaaacactctccgtatctgcgtaccttcatccagaacccgaatcatatcggctgtcacacctacgataaagcggatatcctgttcggcggtaccttcgatatcgagcgcgaactgatccagctgctggctattgacgtgctgaacggtaacgacgaagagttcgacggttacgttactcaaggcggtacggaggcgaacatccaggcgatgtgggtttaccgcaactatttcaagaaggaacgtaaagctaaacacgaagaaattgcgattatcaccagcgcggacacccactatagcgcctacaaaggttccgatctgctgaacatcgatatcatcaaagtgccggtagacttctactcccgcaaaatccaggaaaacaccctggattccatcgtcaaggaagcaaaagaaatcggcaaaaagtacttcattgtcatttccaacatgggtacgaccatgtttggcagcgtggacgacccggatctgtacgcaaacatctttgacaaatataacctggagtacaaaatccacgtcgatggtgcttttggtggtttcatctacccaattgacaacaaagagtgcaaaaccgatttctctaacaaaaacgtctcctctatcacgctggacggccacaaaatgctgcaagctccgtacggtaccggtatttttgtgtctcgcaaaaacctgatccataacaccctgaccaaagaagctacctacatcgaaaatctggatgttaccctgtctggttcccgttctggtagcaacgccgtagcgatctggatggtgctggcgtcctacggcccgtatggctggatggagaaaatcaataaactgcgcaaccgtaccaaatggctgtgcaaacaactgaatgacatgcgtatcaaatactacaaggaggactccatgaacatcgtaaccattgaagagcagtatgtcaacaaagaaatcgctgagaagtacttcctggtgccagaagttcataacccgacgaataactggtacaaaattgttgtgatggaacacgtggaactggatatcctgaactctctggtgtacgatctgcgtaagttcaacaaagaacacctgaaagctatgtaa

SEQ ID NO: 23 - DNA, 1086 bp, unidentified
  misc_feature 1..1086: note = PsiK; kinase from Psilocybe cyanescens
  source 1..1086: mol_type = genomic DNA
atgacgttcgatctgaaaactgaagagggtctgctgagctatctgactaaacatctgtctctggatgttgcgccgaacggtgtcaaacgtctgagcggcggtttcgttaacgtcacttggcgtgttggtctgaacgcaccgtaccacggccatactagcatcattctgaaacatgcccagccgcatctgtcctccgatatcgacttcaaaatcggtgtggaacgttctgcctacgaataccaggcgctgaaaatcgttagcgcgaacagctctctgctgggttccagcgacatccgcgtgagcgtgccggaaggtctgcactacgatgtggttaacaacgcactgatcatgcaggatgtaggcacgatgaaaaccctgctggattatgttactgcgaaacctccgatctctgcggagatcgcgagcctggttggctcccaaattggtgctttcatcgcccgtctgcacaacctgggtcgtgaaaacaaagacaaagacgatttcaaattcttcagcggcaacatcgttggccgtaccaccgctgaccagctgtaccaaactatcatcccgaacgcagcaaagtatggcatcgacgacccgatcctgccgattgtggttaaagaactggttgaagaagttatgaacagcgaagaaaccctgatcatggcggatctgtggtctggcaacatcctgctgcagttcgacgagaacagcacggaactgactcgcatctggctggtagactgggaactgtgcaaatacggcccgccgtctctggatatgggctactttctgggcgactgtttcctggtcgcgcgcttccaggaccagctggtaggcaccagcatgcgtcaggcttacctgaaatcttatgctcgtaacgttaaggaaccgatcaactacgcaaaagccactgccggtatcggcgcgcacctggtcatgtggaccgatttcatgaagtggggcaacgacgaggagcgtgaagagttcgtaaagaaaggcgttgaagcgttccatgaagctaacgaagataaccgcaacggcgagatcacttccatcctggttaaagaagcatcccgtacctaa

SEQ ID NO: 24 - DNA, 1089 bp, unidentified
  misc_feature 1..1089: note = PsiK; based on kinase from Psilocybe cubensis
  source 1..1089: mol_type = genomic DNA
atggctttcgacctgaagactgaagatggcctgattacttacctgaccaaacacctgagcctggacgtagatacctctggcgtaaaacgtctgtctggcggtttcgtgaacgttacctggcgtatcaaactgaacgcgccgtaccagggtcatacctctatcatcctgaaacacgcgcagccgcacatgtccaccgacgaagatttcaaaattggcgtcgaacgttctgtgtacgaataccaggctattaaactgatgatggcgaaccgcgaagtactgggtggtgttgacggcatcgtatccgtaccagaaggcctgaactatgacctggagaacaacgcactgatcatgcaagatgtgggtaaaatgaaaaccctgctggattatgttacggcgaaaccgccactggcaaccgatatcgcgcgtctggtcggtactgaaattggtggtttcgtagcgcgcctgcacaacatcggtcgtgaacgtcgtgatgatccggagtttaaattcttctccggtaacatcgtgggtcgtaccacctctgaccaactgtaccagaccatcattccgaacgcggcgaaatacggtgtggacgatccgctgctgccgactgttgtgaaagatctggttgacgacgtcatgcacagcgaagaaaccctggttatggccgatctgtggtctggtaacatcctgctgcaactggaagagggtaacccgtccaagctgcagaagatttacattctggactgggaactgtgtaaatacggtccggcatctctggatctgggctacttcctgggcgattgctatctgatctctcgtttccaggacgaacaggttggtactactatgcgtcaggcttacctgcagagctacgcgcgtacgtccaaacattctatcaactatgctaaagtaaccgcaggcatcgcggcgcacatcgttatgtggaccgacttcatgcaatggggctctgaagaggaacgtatcaactttgttaaaaagggtgtggcagcgttccacgacgctcgtggtaacaacgataacggcgaaatcactagcaccctgctgaaggaaagcagcaccgcttaa

SEQ ID NO: 25 - DNA, 927 bp, unidentified
  misc_feature 1..927: note = PsiM; based on 4-hydroxytriptamine methylase from P. cyanescens
  source 1..927: mol_type = genomic DNA
atgattcgtaatccgtatcgtgatggcgtagactaccaagctctggcagaagcattcccggctctgaaaccacatgtgaccgtaaactccgataacaccacttctatcgacttcgcggttcctgaagcacagcgcctgtacaccgcagctctgctgcaccgcgatttcggcctgactatcactctgccggaggatcgtctgtgcccgactgttccgaaccgtctgaactacgttctgtgggtggaagacattctgaaagttacttctgatgctctgggcctgcctgataaccgtcaggtcaaaggcattgatatcggtaccggtgcgtccgctatttacccgatgctggcttgctctcgttttaaaacttggagcatggttgcgacggaagttgaccaaaaatgtattgacaccgctcgcctgaacgtgatcgcgaacaacctgcaggaacgtctggcaatcattgcaaccagcgttgacggtcctattctggtgccgctgctgcaggcgaactctgatttcgagtacgacttcaccatgtgcaaccctccgttttacgacggtgcgtctgacatgcagactagcgatgcagctaaaggtttcggctttggcgttaacgcaccgcacaccggcaccgtgctggaaatggcaactgaaggcggcgagtctgcgttcgtagcgcagatggtgcgtgaaagcctgaacctgcagactcgttgccgttggttcacttctaacctgggtaagctgaaatccctgtacgaaatcgttggtctgctgcgtgaacatcagatttctaactatgcgatcaacgaatacgtacagggtgcaacccgtcgctatgcaatcgcgtggagcttcatcgacgtgcgtctgccggatcacctgtcccgtccgtctaacccggatctgagctccctgttctaa

SEQ ID NO: 26 - DNA, 927 bp, unidentified
  misc_feature 1..927: note = PsiM; based on 4-hydroxytriptamine methylase from P. cubensis
  source 1..927: mol_type = genomic DNA
atgatccgcaatccttaccgtactccgatcgattatcaagcactgagcgaagccttcccgccgctgaaaccgttcgtttctgtgaacgcagacggcacttcttccgtcgatctgactattccagaagcacagcgcgccttcacggcggcgctgctgcatcgcgacttcggtctgaccatgactatcccggaggatcgcctgtgtccgaccgttccgaaccgtctgaactatgtgctgtggatcgaagacatcttcaactacaccaacaagactctgggtctgagcgacgatcgtccgatcaaaggtgtggacattggtaccggcgcctccgctatctacccaatgctggcgtgtgcgcgtttcaaggcatggtctatggttggcactgaggtggaacgcaaatgtattgacactgctcgcctgaacgtggtagcaaacaacctgcaggaccgtctgagcatcctggaaacctccatcgatggtcctattctggttccgatctttgaagcgaccgaagaatacgaatatgaatttactatgtgtaacccgccgttctacgacggcgcagccgatatgcagacgtctgacgcggctaagggtttcggcttcggcgtaggtgccccgcactctggcactgtgatcgagatgtccaccgaaggcggtgagtccgcctttgttgcccagatggtgcgtgaatctctgaaactgcgcacccgttgccgttggtatacctctaacctgggcaaactgaaatctctgaaagaaatcgtgggcctgctgaaagaactggaaatctccaactatgcgattaacgaatacgttcagggttctactcgtcgttacgcggttgcatggagcttcaccgatatccagctgccggaagaactgtcccgcccgtccaacccggaactgtcctctctgttctaa

SEQ ID NO: 27 - DNA, 792 bp, unidentified
  misc_feature 1..792: note = rINMT; based on rabbit lung INMT enzyme
  source 1..792: mol_type = genomic DNA
atggagggcggctttactggtggtgacgaatatcaaaaacatttcctgccgcgtgactacctgaacacctactactctttccaaagcggcccgtctccggaagcagagatgctgaaatttaacctggaatgcctgcacaaaacctttggcccgggtggcctgcagggtgacactctgatcgacattggctctggcccgactatctatcaggtactggccgcctgtgaatccttcaaagacatcaccctgagcgacttcaccgatcgtaaccgtgaagagctggccaagtggctgaagaaagagccgggtgcctatgattggactccggcactgaaatttgcctgcgaactggaaggcaacagcggtcgttggcaggaaaaagccgaaaaactgcgcgcaaccgtaaaacgcgttctgaaatgtgacgccaatctgtctaacccgctgaccccagttgttctgccaccagccgactgtgtcctgaccctgctggctatggaatgtgcttgttgttccctggatgcgtaccgcgctgccctgcgcaacctggcttctctgctgaaaccgggtggtcacctggtgaccaccgttaccctgcagctgtcttcttacatggttggcgaacgtgaattttcctgcgtcgctctggagaaagaagaagtggagcaggccgtgctggatgcaggctttgatattgaacagctgctgtacagcccgcaatcctacagcgcttctaccgccccgaatcgcggcgtttgctttctggttgcgcgtaagaagccgggctcctaa

SEQ ID NO: 28 - DNA, 792 bp, unidentified; identical to SEQ ID NO: 27
  misc_feature 1..792: note = rINMT-h; with his tag removed
  source 1..792: mol_type = genomic DNA

SEQ ID NO: 29 - DNA, 795 bp, unidentified
  misc_feature 1..795: note = sINMT; based on Spider (Araneus ventricosus) INMT
  source 1..795: mol_type = genomic DNA
atggaaaacgacgaagtgaaaatcaaggaagactacctgaaaaatctggaccctaagaaattccaggaatttaaactggaaggtctgaaggcagaaaaatctgaaaacaactacgatttttggttcaaatgcctgcatgagttctttagcagcggtaactgttccggtacccgcatgctggaaattggttccggtccgaccgtacataacatcgccaccgcatctgcctatttcccgtacatcgtgcaatccgaatttgtcgaatctaattgtgaacagctgcgcaagtggctgcgcaaggacccggattctctggattggaccattttcctgcaacgtgtagcacgtgcagaaggtaaggaagatgttgacgccgcggtcccagagatcgaagatcgtatccgttctgcggttaaagcagtggtccatgcagatgttctggacgaccaagtcatcccgaaagaatatacgcaggaaccgtatgatctgattctgagcgtactgaccttcgaaaccgcggctgttaatctggaatcctattccaagattctgggccgtgctaatcgtctgctgcgcaagggcggtaaaatgattctgatttctgcaactaaatgtacctactggcaggttggcggtaacaaattccactgcctgtccctggaagaacacgaaattgaagatgcactgaaaaagaatggttttggcgagatctgctggaaaatccgtgaacgtccggacgatgatctgccgttcaactgcaagaacgtatactttctgacctgtacgaaactgtaa

SEQ ID NO: 30 - DNA, 678 bp, unidentified
  misc_feature 1..678: note = wINMT; based on Roundworm (Anyclostoma ceylanicum) INMT
  source 1..678: mol_type = genomic DNA
atgtctctgttcgcactgccagtttttgcacacactattcagaagaccatgccggcggaagaacgccagaccctgctggacattggcgcaggcccgacggtgtattccgccctgtgctttcgcgaggtggtaaaacgtgtctacctgtccgactacctgtccaaaaacctggacatcctgaaagcctggcgttccaacaccagcgcttacgattggaaaccgactattaaagtcatcctgcgtaccgagggcatgctgccagcaagcgattccgaaatggaagaagtggaagaacgtgcgcgtgcagcgctgaagtgcggcggtatcctgtacgccaacgtacatgaagacccggtagttccggatatgcgcggcgaacaggttgatgtactggtgaccattttcaccctggaaagcgcgtgtaaatcttacaccgagtattgtcagtgcgtaaaaaacatgctgaagcacctgcgcccaggtggccgcctggttctgggtagcgtgctggaagaggaatcttacaactccggtaaagacgtgatcttccacctgctgcatctgtccgaagatcagattctgggcgcactgggctctgctggtattgatctgaatagcgtcaagaaatacgttctggatgaagatggcgtaatgttcctgatggctgttaagaactaa

SEQ ID NO: 31 - DNA, 783 bp, unidentified
  misc_feature 1..783: note = fINMT; based on American bullfrog (Lithobates catesbeianus) INMT
  source 1..783: mol_type = genomic DNA
atggactctactacccacaaactgtatcacgttcacggtatggattcccgtggcttcctggatacgtacttttctaacaaagaggacatggttttcgccgaagactccatcaaatttccaatggccatgttccactatcagttctccagcggccgcatcgagggcactttcctgatcgacatctctctgggttccgttattcacaacctgtactctgcttccaacagcttcaaaaacatcgttattctgaaatttcaagaaaaatgtatcatggaactgaaccgttggctgcatgatcgtaccggtgcttatgactggagccacacctcctctgcggctgcggaactggagggcacccgtgatcagctgcaagaaaaggaaatgtgcctgcgtagcagcatccgccagatcctgaaatgcgacttcgagcaggaaaacaccattagcccgatgctgctgccgctggctgactgcattatcagcgcgtggattctggaggtaatcagcaaaaacgaagatgaatacatgaaaaatctggagaaaatcatcaaactgctgaaaccgggtggccagctgctgctgattgcatccctggatgcaacctattggtttgctggtgcaggtcgctttcactgcttcaaaattaacgaagaatttgtcaaaaacgcgtttggcaagctgggtctggtgatcgactattacgccgtgcagcgtcgtcgtaacgttagcgacattatcgactataaggccatgctgttcatcgttggccgcaaaggcgagtaa

SEQ ID NO: 32 - DNA, 699 bp, unidentified
  misc_feature 1..699: note = E. coli mtnN
  source 1..699: mol_type = genomic DNA
atgaagattggtatcattggcgcgatggaagaggaagtaaccctgctgcgcgacaagatcgaaaaccgtcagactatttctctgggtggttgcgaaatctacaccggccaactgaacggcacggaagttgctctgctgaaatccggtattggtaaagtcgcggcagcactgggtgcgactctgctgctggaacactgcaaaccggacgtgattatcaacaccggcagcgcaggtggcctggcaccgaccctgaaagtgggcgacatcgtggtgtctgacgaagcgcgttatcacgatgctgacgtgaccgctttcggttatgagtatggtcaactgcctggctgcccggcaggtttcaaagcagacgataaactgattgcggcggctgaagcctgtatcgcagaactgaacctgaacgctgtgcgcggcctgattgtttctggtgacgcttttattaatggtagcgttggcctggccaaaattcgtcacaacttcccgcaggcaatcgctgtagaaatggaagctaccgcaattgcgcacgtatgtcacaacttcaacgttccgtttgtagtggtgcgcgccatctccgatgtcgctgatcagcagtctcacctgagcttcgacgaatttctggcagtagctgctaaacagagctccctgatggttgagtctctggtacagaaactggcccacggctaa

SEQ ID NO: 33 - DNA, 1524 bp, unidentified
  misc_feature 1..1524: note = psiH
  source 1..1524: mol_type = genomic DNA
atgattgtactgctggtatctctggttctggctggctgtatttactacgcaaacgcacgccgtgtgcgtcgcagccgcctgccaccgggccctccgggcatcccgctgccgtttattggcaacatgttcgatatgccttccgagtctccgtggctgcgtttcctgcagtggggtcgtgactaccacactgacatcctgtacctgaacgcaggtggtaccgaaatcattatcctgaacacgctggatgcaatcaccgatctgctggaaaagcgtggctctatgtattccggtcgtctggaatccactatggttaacgagctgatgggctgggagttcgacctgggtttcattacctatggtgaacgttggcgcgaagaacgtcgtatgttcgctaaagagttcagcgaaaagaacattcgtcagttccgtcatgcgcagattaaagcggcaaaccagctggtgcgtcagctgatcaaaactccggaccgttggtcccagcatattcgtcaccagattgccgcaatgtctctggatatcggttatggcatcgacctggcagaagatgatccgtggatcgcggcaacccagctggctaacgaaggcctggcggaagcctctgtaccgggcagcttctgggtcgatagcttcccggctctgaaatatctgccgtcctggctgccgggtgcgggtttcaaacgcaaagcaaaagtgtggaaagaaggcgcagaccacatggtaaatatgccatacgaaactatgaagaaactgactgttcagggtctggctcgcccaagctacgcaagcgctcgtctgcaagcgatggacccagatggcgatctggaacaccaggaacacgttattcgtaacacggctaccgaggttaatgtgggtggtggtgatactaccgtttctgccgtgtccgccttcattctggcaatggtcaaatacccggaagtgcagcgtcaggttcaggctgaactggacgctctgaccagcaaaggtgttgtacctaactatgacgaagaagacgacagcctgccgtacctgaccgcctgtgttaaagagatttttcgctggaatcagattgcaccgctggctatcccgcaccgtctgatcaaagacgacgtttatcgcggttatctgatcccgaaaaacgccctggtgtacgctaacagctgggcggttctgaatgatccggaagaatacccgaacccgagcgagtttcgtccggaacgttacctgtcttctgacggtaaaccggacccaactgtgcgtgacccgcgcaaagcggcgttcggttatggtcgtcgtaactgtccgggtatccacctggcacagtctaccgtttggattgctggcgcaactctgctgtctgtgtttaacatcgaacgcccggttgatggcaacggcaaaccgatcgacattccggctaccttcaccaccggtttcttccgtcatccggaaccgtttcaatgtcgtttcgtgccgcgcacccaagagatcctgaaatccgtgagcggttaa

SEQ ID NO: 34 - DNA, 1986 bp, unidentified
  misc_feature 1..1986: note = chuA gene; encoding hemin receptor from Escherichia coli O157H7str. EDL933
  source 1..1986: mol_type = genomic DNA
atgtctcgtccacaatttacttctctgcgtctgtctctgctggcactggctgtgtccgccactctgcctacgtttgcgttcgctactgaaaccatgactgttactgcgactggtaatgcgcgctcttccttcgaagccccgatgatggtttctgtaattgacacctctgctccggaaaaccagaccgctacttccgcgaccgacctgctgcgccacgttccgggcattaccctggatggcacgggtcgtactaacggtcaagatgtgaacatgcgtggttacgaccaccgcggcgtactggtgctggttgatggtgttcgccagggtacggatactggccacctgaacggtaccttcctggacccggctctgatcaagcgcgttgagatcgtacgtggcccgagcgcgctgctgtacggttctggcgcgctgggcggcgttatcagctacgatactgttgacgcgaaggatctgctgcaggaaggtcagtccagcggtttccgtgtattcggcacgggtggtaccggcgatcactccctgggcctgggtgcttctgctttcggccgcactgaaaacctggatggcatcgttgcatggtcttctcgcgatcgcggtgacctgcgtcagtccaatggcgaaactgcaccgaacgacgaatctattaacaacatgctggcaaaaggtacgtggcagatcgactccgcgcagagcctgtctggcctggtgcgctattataacaacgatgctcgtgaaccgaaaaacccgcagacggttggcgcgtccgaaagcagcaatccgatggttgaccgttccactatccagcgtgacgcgcagctgtcctacaaactggcaccgcaaggtaacgattggctgaacgctgatgccaagatttactggtccgaagtacgtatcaatgcacaaaacacgggctcttctggcgagtaccgcgaacaaattaccaaaggcgctcgtctggagaatcgctctaccctgttcgccgacagcttcgcttctcacctgctgacttacggtggtgaatattaccgtcaggaacagcaccctggtggcgctaccaccggcttccctcaggcaaagatcgacttcagcagcggttggctgcaggatgaaatcaccctgcgcgacctgccgatcaccctgctgggcggtacccgttatgattcttaccgtggttcctccgatggctacaaagacgttgacgcggataaatggtcttcccgtgctggtatgaccattaacccgacgaactggctgatgctgttcggttcctacgcgcaagccttccgtgctccgaccatgggcgaaatgtacaatgactctaaacacttctctatcggtcgcttctacaccaactattgggtgccgaacccgaatctgcgtccggagactaacgaaacccaagagtacggcttcggtctgcgcttcgatgacctgatgctgtccaacgacgccctggaatttaaagcctcttacttcgataccaaagcaaaagactatatctccaccactgtcgatttcgcggcagcaaccactatgtcttacaacgttccgaacgcgaaaatctggggttgggatgttatgaccaagtacaccactgacctgttctctctggacgtagcgtataatcgcactcgtggcaaagacaccgataccggcgaatacatcagctccattaacccggacaccgtaacttctaccctgaacatcccaatcgcgcactccggcttctctgttggttgggtgggcacgttcgcggatcgttccactcacatttcctcttcttacagcaaacagccgggttatggtgtgaacgacttctatgttagctaccagggtcagcaggctctgaagggcatgaccactactctggtcctgggtaacgcgttcgataaggaatactggagccctcagggtattccgcaggatggccgtaacggcaaaattttcgtgtcttatcaatggtggtaa

SEQ ID NO: 35 - DNA, 1524 bp, unidentified; identical to SEQ ID NO: 33
  misc_feature 1..1524: note = rCPR gene
  source 1..1524: mol_type = genomic DNA

SEQ ID NO: 36 - DNA, 2250 bp, unidentified
  misc_feature 1..2250: note = pCPR gene; Psilocybe cyanescens uncharacterized gene CVT25_015047with homology to NADPH cytochrome P450 reductase
  source 1..2250: mol_type = genomic DNA
atgaccgatcctaaccgtactactttctcttctgctctgcacccgctggcagttgtatctatggcgtccagctccagcgacgttttcgtgctgggcctgggcgttgttctggcggcgctgtatattttccgtgatcagctgtttgcagcatctaaaccaaaggtggccccggtttccaccaccaaaccggcgaacggcagcgcgaacccgcgtgacttcatcgctaaaatgaaacaaggtaagaaacgtatcgttatcttctacggcagccagaccggtactgctgaagagtatgctatccgtctggcgaaagaagcgaaacaaaaattcggtctggcgtctctggtttgtgatccggaggaatatgatttcgagaaactggatcagctgccggaagactccattgcgttcttcgtggttgcgacctacggcgaaggcgagcctacggataacgcggtgcaactgctgcagaacctgcaggacgactccttcgaattttctaacggtgaacgcaaactgtctggcctgaaatacgttgtattcggtctgggcaataagacttacgagcactataacctgatcggtcgtaccgtagacgctcagctggcgaaaatgggcgcagtgcgtgtgggtgaacgcggcgaaggtgacgatgacaaaagcatggaagaagattatctggagtggaaagatggtatgtgggacgcgttcgcagcagcaatgggtgtcgaagaaggtcagggtggtgacagcgctgatttcgttgtgtccgaactggaaagccacccgccagaaaaagtttacctgggtgaatactctgcgcgcgctctgactaaaaccaaaggtattcatgatgccaaaaatccgctggctgccccgatcactgtggcacgcgaactgtttcagagcgtagtggaccgcaactgtgttcacgtggagttcaacattgaaggttctggcattacgtaccaacacggcgatcacgttggtctgtggccgctgaacccggatgttgaagttgaacgtctgctgtgtgtgctgggtctgaccgagaaacgcgatgcggttattagcatcgaatctctggacccggcactggcaaaagttccgtttccggtgccgaccacctacgcggcagttctgcgtcactatattgatgtttctgcggtcgcaggccgtcagatcctgggtactctgtccaaattcgcgccgaccccggaagcggaagcgtttctgaaaaacctgaataccaacaaagaggaataccataatgtggttgcgaacggttgtctgaaactgggtgaaatcctgcaggttgctacgggtaacgatatcactgtagctccaacccctggtaacactaccaaatggccgattcctttcgacattatcgtaagcgccattccgcgtctgcagccgcgttattacagcatcagctcttcccctaaagttcacccgaacactatccatgcgaccgtggttgtcctgaaatacgagaacgtaccgaccgatcctatcccgcgtaagtgggtgtatggtgtaggtagcaatttcctgctgaacctgaaacatgcgattaacaaagagccggtcccgttcatcacgcagaatggtgaacagcgcgttggtgttccggaatatctgattgctggtccgcgtggcagctacaaaactgaatctcacttcaaagcgccgattcacgtacgtcgcagcacgttccgcctgccgactaaccctaaaagcccggttattatgattggtccgggtactggtgttgcaccgttccgtggtttcgtgcaggaacgtgttgcgctggctcgtcgttctgtggagaaaaacggtccggaatctctgaatgactggggccgcatctccctgttctacggctgccgccgtagcgacgaagatttcctgtacaaagacgaatggccgcagtaccaagaagaactgaaaggtaaattcaaactgcattgcgcattctctcgcgaaaactacaaaccggatggtagcaaaatctatgtgcaggacctgatctgggaggatcgcgagcatatcgcggacgccatcctgaatggcaagggttacgtatacatttgcggtgaggcgaaatctatgagcaaacaagtcgaggaggttctggcacgtatcctgggcgaggcgaaaggcggctccggtgctgttgaaggcgtggctgaaatcaaactgctgaaagagcgttcccgtctgatgctggattacgaactggcctaa

SEQ ID NO: 37 - DNA, 2241 bp, unidentified
  misc_feature 1..2241: note = aCPR gene; Agrocybe pediades uncharacterized gene with homologyto NADPH cytochrome P450 reductase
  source 1..2241: mol_type = genomic DNA
atgtcctattctaaccattctaccgcctttccaaatccgctgtctgcgtacaacatggcatccagcagctccgacgtcttcattctgggcctgggcatcgtactggcagcactgtacatcttccgtgatcagctgtttgcggccagcaaaccgaaagtggcgcctgtagccgcctccaaagcggcgaacggtagcggcaacccgcgtgatttcatcgccaaaatgaaagaaggcaagaaacgcatcgtgatcttctacggttcccagaccggcactgctgaagaatacgcgatccgtctggctaaagaggctaaacagaaatttggtctggcgtctctggtttgcgatccagaagaatacgattttgagaacctggatcaactgccggaagactgcgctgctttcttcgtgatggcaacgtacggcgaaggtgagccgactgataacgcagtacaactgatgcagaacctgcaggatgaatccttcgaattttctaacggtgaacgtaaactggaaggcctgaagtacgtggtattcggcctgggtaacaaaacctatgaacactacaacctgattggtcgtaatgtagatgctgccctgtccaaaatgggtgccatccgcatcggcgaacgtggtgaaggtgatgatgataaaagcatggaggaggactacctggaatggaaagacggcatgtgggaggcattcgcaaccgccatgaacgtggaggaaggtcagggcggtgatagcgcagacttcgtagtgtctgaactggaaagccaccctgaagataaagtgtacctgggtgaatactctgctcgcgccctgacgcgcaccaaaggtatccacgacgctaaaaacccgtatgctgcaccgatcgtcgttgcacgcgaactgttctctgctaacgccgatcgtaactgcgttcatatcgaatttaacacggaaggttctggtattacgtaccagcacggcgaccacgttggtctgtggccgctgaacccggatgtagaagtcgagcgtctgctgtgcgttctgggcctgacccagaaaaaggacaccgttatcggcatcgaaagcctggacccggcgctggctaaggtaccattcccggttcctactacctacggcaccgttctgcgccactacattgatatcagcgctgttgcgggccgtcagatcctgggcaccatgtccaaattcgctcctactcctgaagcggaagccttcctgaaaaacctgaacaccaacaaagaagaataccaccgtgttgtccacgacggttgcctgaaactgggtgaggttctgcaactggcagcgggtaacaacatcaaagcgattccgacttccgagaataccactcagtggaagatcccgttcgatattatcgtgtccgcaatccctcgtctgcagccgcgttactactccatttcttcttccccgaaactgcatccgaacagcattcatgcgacggctgttgtgctgaaatatcaaaatgttccgtccgagctggtaacttctaagtgggtgtacggtgtgggctccaacttcctgctgagcctgaaacatgcaagcaacaacgaaccgatcccagtgatctccgaaaacggcgaagagcgtgttgcagttccggcctatctgatcgaaggcccgcgtggcagctataaaaccgaaacccactacaaagcaccgatccacgtgcgtcgttctacctttcgtctgccgaccaacccgaagagcccggtaatcatggtgggtccgggcaccggcgtggcaccgtttcgtggcttcgtacaggaacgcgttgcactggcacgccgttccatcgaaaagaacggtccggatgcgctgaatgactggggccgcatctctctgttctatggttgccgtcgtgaggacgaggactttctgtataaagacgaatgggcaaaatacgaagaagaactgaaaggtaaatttaaaatgcactgtgcatttagccgtcagaactacaaaccggatggttctaagatctacgtacaggacctgatttgggaggaccgtgaacatatcgcggacgctatcctgaacggcaaaggttatgtctacatttgcggtgaggcaaaaagcatgtctaaacaggttgaagaggtactggctcgtatcctgggtgaagctaaaggcggctctgccgaagtcgaaggtgcggctgaaattaaactgctgaaagaacgctctcgtctgatgctggatgtttggagctaa

SEQ ID NO: 38 - DNA, 1107 bp, unidentified
  misc_feature 1..1107: note = malE' gene
  source 1..1107: mol_type = genomic DNA
atgaaaatcgaagaaggtaaactggttatctggatcaatggtgataaaggttataacggcctggcggaagtaggtaaaaagttcgaaaaagataccggcatcaaggtaaccgtggaacatccagacaaactggaagaaaagttcccgcaagttgctgctactggtgacggcccggatatcatcttctgggcgcacgaccgtttcggcggctacgcacaatctggcctgctggctgaaatcaccccggataaagcattccaggataaactgtacccgttcacctgggacgcggtgcgctacaatggcaaactgatcgcctacccgatcgccgttgaagcgctgtctctgatctacaacaaggacctgctgccgaacccgccgaagacctgggaagaaatccctgccctggacaaggaactgaaggctaagggtaaaagcgcgctgatgttcaacctgcaggaaccgtacttcacttggccactgattgctgctgatggcggttatgctttcaaatacgagaacggcaagtacgacattaaagacgtgggtgtggacaacgcaggcgcaaaagcaggtctgaccttcctggtggatctgatcaagaacaagcacatgaatgccgatactgactactccattgccgaggcggcttttaacaaaggcgaaactgctatgacgatcaacggtccttgggcttggagcaatatcgatacttccaaagttaactacggtgtaaccgttctgccaacgttcaagggccagccgtctaaaccgtttgtcggtgtgctgtctgctggtatcaacgcagcttctccgaacaaagaactggcaaaagaatttctggaaaattatctgctgaccgacgaaggtctggaagcagttaacaaagataaaccgctgggcgcggtagcgctgaaaagctacgaagaggagctggctaaggacccgcgtatcgccgcaaccatggagaacgcgcagaaaggcgaaattatgcctaacattccgcagatgtctgccttttggtacgcggtgcgtaccgccgttatcaacgccgcgtccggtcgtcagacggttgatgaagctctgaaagatgcacagactgtgtaa

SEQ ID NO: 39 - protein, 439 aa, unidentified
  REGION 1..439: note = psiD
MQVIPACNSAAIRSLCPTPESFRNMGWLSVSDAVYSEFIGELATRASNRNYSNEFGLMQPIQEFKAFIESDPVVHQEFIDMFEGIQDSPRNYQELCNMFNDIFRKAPVYGDLGPPVYMIMAKLMNTRAGFSAFTRQRLNLHFKKLFDTWGLFLSSKDSRNVLVADQFDDRHCGWLNERALSAMVKHYNGRAFDEVFLCDKNAPYYGFNSYDDFFNRRFRNRDIDRPVVGGVNNTTLISAACESLSYNVSYDVQSLDTLVFKGETYSLKHLLNNDPFTPQFEHGSILQGFLNVTAYHRWHAPVNGTIVKIINVPGTYFAQAPSTIGDPIPDNDYDPPPYLKSLVYFSNIAARQIMFIEADNKEIGLIFLVFIGMTEISTCEATVSEGQHVNRGDDLGMFHFGGSSFALGLRKDCRAEIVEKFTEPGTVIRINEVVAALKA

SEQ ID NO: 40 - protein, 490 aa, unidentified
  REGION 1..490: note = rumD Ruminococcus gnavus
MSQVIKKKRNTFMIGTEYILNSTQLEEAIKSFVHDFCAEKHEIHDQPVVVEAKEHQEDKIKQIKIPEKGRPVNEVVSEMMNEVYRYRGDANHPRFFSFVPGPASSVSWLGDIMTSAYNIHAGGSKLAPMVNCIEQEVLKWLAKQVGFTENPGGVFVSGGSMANITALTAARDNKLTDINLHLGTAYISDQTHSSVAKGLRIIGITDSRIRRIPTNSHFQMDTTKLEEAIETDKKSGYIPFVVIGTAGTTNTGSIDPLTEISALCKKHDMWFHIDGAYGASVLLSPKYKSLLTGTGLADSISWDAHKWLFQTYGCAMVLVKDIRNLFHSFHVNPEYLKDLENDIDNVNTWDIGMELTRPARGLKLWLTLQVLGSDLIGSAIEHGFQLAVWAEEALNPKKDWEIVSPAQMAMINFRYAPKDLTKEEQDILNEKISHRILESGYAAIFTTVLNGKTVLRICAIHPEATQEDMQHTIDLLDQYGREIYTEMKKA

SEQ ID NO: 41 - protein, 417 aa, unidentified
  REGION 1..417: note = cloD Clostridium sporogenes
MKFWRKYTQQEMDEKITESLEKTLNYDNTKTIGIPGTKLDDTVFYDDHSFVKHSPYLRTFIQNPNHIGCHTYDKADILFGGTFDIERELIQLLAIDVLNGNDEEFDGYVTQGGTEANIQAMWVYRNYFKKERKAKHEEIAIITSADTHYSAYKGSDLLNIDIIKVPVDFYSRKIQENTLDSIVKEAKEIGKKYFIVISNMGTTMFGSVDDPDLYANIFDKYNLEYKIHVDGAFGGFIYPIDNKECKTDFSNKNVSSITLDGHKMLQAPYGTGIFVSRKNLIHNTLTKEATYIENLDVTLSGSRSGSNAVAIWMVLASYGPYGWMEKINKLRNRTKWLCKQLNDMRIKYYKEDSMNIVTIEEQYVNKEIAEKYFLVPEVHNPTNNWYKIVVMEHVELDILNSLVYDLRKFNKEHLKAM

SEQ ID NO: 42 - protein, 361 aa, unidentified
  REGION 1..361: note = PsiK based on kinase from Psilocybe cyanescens
MTFDLKTEEGLLSYLTKHLSLDVAPNGVKRLSGGFVNVTWRVGLNAPYHGHTSIILKHAQPHLSSDIDFKIGVERSAYEYQALKIVSANSSLLGSSDIRVSVPEGLHYDVVNNALIMQDVGTMKTLLDYVTAKPPISAEIASLVGSQIGAFIARLHNLGRENKDKDDFKFFSGNIVGRTTADQLYQTIIPNAAKYGIDDPILPIVVKELVEEVMNSEETLIMADLWSGNILLQFDENSTELTRIWLVDWELCKYGPPSLDMGYFLGDCFLVARFQDQLVGTSMRQAYLKSYARNVKEPINYAKATAGIGAHLVMWTDFMKWGNDEEREEFVKKGVEAFHEANEDNRNGEITSILVKEASRT

SEQ ID NO: 43 - protein, 362 aa, unidentified
  REGION 1..362: note = PsiK based on kinase from Psilocybe cubensis
MAFDLKTEDGLITYLTKHLSLDVDTSGVKRLSGGFVNVTWRIKLNAPYQGHTSIILKHAQPHMSTDEDFKIGVERSVYEYQAIKLMMANREVLGGVDGIVSVPEGLNYDLENNALIMQDVGKMKTLLDYVTAKPPLATDIARLVGTEIGGFVARLHNIGRERRDDPEFKFFSGNIVGRTTSDQLYQTIIPNAAKYGVDDPLLPTVVKDLVDDVMHSEETLVMADLWSGNILLQLEEGNPSKLQKIYILDWELCKYGPASLDLGYFLGDCYLISRFQDEQVGTTMRQAYLQSYARTSKHSINYAKVTAGIAAHIVMWTDFMQWGSEEERINFVKKGVAAFHDARGNNDNGEITSTLLKESSTA

SEQ ID NO: 44 - protein, 308 aa, unidentified
  REGION 1..308: note = PsiM based on 4-hydroxytriptamine methylase from P. cyanescens
MIRNPYRDGVDYQALAEAFPALKPHVTVNSDNTTSIDFAVPEAQRLYTAALLHRDFGLTITLPEDRLCPTVPNRLNYVLWVEDILKVTSDALGLPDNRQVKGIDIGTGASAIYPMLACSRFKTWSMVATEVDQKCIDTARLNVIANNLQERLAIIATSVDGPILVPLLQANSDFEYDFTMCNPPFYDGASDMQTSDAAKGFGFGVNAPHTGTVLEMATEGGESAFVAQMVRESLNLQTRCRWFTSNLGKLKSLYEIVGLLREHQISNYAINEYVQGATRRYAIAWSFIDVRLPDHLSRPSNPDLSSLF

SEQ ID NO: 45 - protein, 308 aa, unidentified
  REGION 1..308: note = PsiM based on 4-hydroxytriptamine methylase from P. cubensis
MIRNPYRTPIDYQALSEAFPPLKPFVSVNADGTSSVDLTIPEAQRAFTAALLHRDFGLTMTIPEDRLCPTVPNRLNYVLWIEDIFNYTNKTLGLSDDRPIKGVDIGTGASAIYPMLACARFKAWSMVGTEVERKCIDTARLNVVANNLQDRLSILETSIDGPILVPIFEATEEYEYEFTMCNPPFYDGAADMQTSDAAKGFGFGVGAPHSGTVIEMSTEGGESAFVAQMVRESLKLRTRCRWYTSNLGKLKSLKEIVGLLKELEISNYAINEYVQGSTRRYAVAWSFTDIQLPEELSRPSNPELSSLF

SEQ ID NO: 46 - protein, 263 aa, unidentified
  REGION 1..263: note = rINMT based on rabbit lung INMT enzyme
MEGGFTGGDEYQKHFLPRDYLNTYYSFQSGPSPEAEMLKFNLECLHKTFGPGGLQGDTLIDIGSGPTIYQVLAACESFKDITLSDFTDRNREELAKWLKKEPGAYDWTPALKFACELEGNSGRWQEKAEKLRATVKRVLKCDANLSNPLTPVVLPPADCVLTLLAMECACCSLDAYRAALRNLASLLKPGGHLVTTVTLQLSSYMVGEREFSCVALEKEEVEQAVLDAGFDIEQLLYSPQSYSASTAPNRGVCFLVARKKPGS

SEQ ID NO: 47 - protein, 263 aa, unidentified; identical to SEQ ID NO: 46
  REGION 1..263: note = rINMT-h

SEQ ID NO: 48 - protein, 264 aa, unidentified
  REGION 1..264: note = sINMT based on Spider (Araneus ventricosus) INMT
MENDEVKIKEDYLKNLDPKKFQEFKLEGLKAEKSENNYDFWFKCLHEFFSSGNCSGTRMLEIGSGPTVHNIATASAYFPYIVQSEFVESNCEQLRKWLRKDPDSLDWTIFLQRVARAEGKEDVDAAVPEIEDRIRSAVKAVVHADVLDDQVIPKEYTQEPYDLILSVLTFETAAVNLESYSKILGRANRLLRKGGKMILISATKCTYWQVGGNKFHCLSLEEHEIEDALKKNGFGEICWKIRERPDDDLPFNCKNVYFLTCTKL

SEQ ID NO: 49 - protein, 225 aa, unidentified
  REGION 1..225: note = wINMT based on Roundworm (Anyclostoma ceylanicum) INMT
MSLFALPVFAHTIQKTMPAEERQTLLDIGAGPTVYSALCFREVVKRVYLSDYLSKNLDILKAWRSNTSAYDWKPTIKVILRTEGMLPASDSEMEEVEERARAALKCGGILYANVHEDPVVPDMRGEQVDVLVTIFTLESACKSYTEYCQCVKNMLKHLRPGGRLVLGSVLEEESYNSGKDVIFHLLHLSEDQILGALGSAGIDLNSVKKYVLDEDGVMFLMAVKN

SEQ ID NO: 50 - protein, 260 aa, unidentified
  REGION 1..260: note = fINMT based on American bullfrog (Lithobates catesbeianus) INMT
MDSTTHKLYHVHGMDSRGFLDTYFSNKEDMVFAEDSIKFPMAMFHYQFSSGRIEGTFLIDISLGSVIHNLYSASNSFKNIVILKFQEKCIMELNRWLHDRTGAYDWSHTSSAAAELEGTRDQLQEKEMCLRSSIRQILKCDFEQENTISPMLLPLADCIISAWILEVISKNEDEYMKNLEKIIKLLKPGGQLLLIASLDATYWFAGAGRFHCFKINEEFVKNAFGKLGLVIDYYAVQRRRNVSDIIDYKAMLFIVGRKGE

SEQ ID NO: 51 - protein, 232 aa, unidentified
  REGION 1..232: note = E. coli mtnN
MKIGIIGAMEEEVTLLRDKIENRQTISLGGCEIYTGQLNGTEVALLKSGIGKVAAALGATLLLEHCKPDVIINTGSAGGLAPTLKVGDIVVSDEARYHDADVTAFGYEYGQLPGCPAGFKADDKLIAAAEACIAELNLNAVRGLIVSGDAFINGSVGLAKIRHNFPQAIAVEMEATAIAHVCHNFNVPFVVVRAISDVADQQSHLSFDEFLAVAAKQSSLMVESLVQKLAHG

SEQ ID NO: 52 - protein, 507 aa, unidentified
  REGION 1..507: note = psiH
MIVLLVSLVLAGCIYYANARRVRRSRLPPGPPGIPLPFIGNMFDMPSESPWLRFLQWGRDYHTDILYLNAGGTEIIILNTLDAITDLLEKRGSMYSGRLESTMVNELMGWEFDLGFITYGERWREERRMFAKEFSEKNIRQFRHAQIKAANQLVRQLIKTPDRWSQHIRHQIAAMSLDIGYGIDLAEDDPWIAATQLANEGLAEASVPGSFWVDSFPALKYLPSWLPGAGFKRKAKVWKEGADHMVNMPYETMKKLTVQGLARPSYASARLQAMDPDGDLEHQEHVIRNTATEVNVGGGDTTVSAVSAFILAMVKYPEVQRQVQAELDALTSKGVVPNYDEEDDSLPYLTACVKEIFRWNQIAPLAIPHRLIKDDVYRGYLIPKNALVYANSWAVLNDPEEYPNPSEFRPERYLSSDGKPDPTVRDPRKAAFGYGRRNCPGIHLAQSTVWIAGATLLSVFNIERPVDGNGKPIDIPATFTTGFFRHPEPFQCRFVPRTQEILKSVSG

SEQ ID NO: 53 - protein, 661 aa, unidentified
  REGION 1..661: note = chuA
MSRPQFTSLRLSLLALAVSATLPTFAFATETMTVTATGNARSSFEAPMMVSVIDTSAPENQTATSATDLLRHVPGITLDGTGRTNGQDVNMRGYDHRGVLVLVDGVRQGTDTGHLNGTFLDPALIKRVEIVRGPSALLYGSGALGGVISYDTVDAKDLLQEGQSSGFRVFGTGGTGDHSLGLGASAFGRTENLDGIVAWSSRDRGDLRQSNGETAPNDESINNMLAKGTWQIDSAQSLSGLVRYYNNDAREPKNPQTVGASESSNPMVDRSTIQRDAQLSYKLAPQGNDWLNADAKIYWSEVRINAQNTGSSGEYREQITKGARLENRSTLFADSFASHLLTYGGEYYRQEQHPGGATTGFPQAKIDFSSGWLQDEITLRDLPITLLGGTRYDSYRGSSDGYKDVDADKWSSRAGMTINPTNWLMLFGSYAQAFRAPTMGEMYNDSKHFSIGRFYTNYWVPNPNLRPETNETQEYGFGLRFDDLMLSNDALEFKASYFDTKAKDYISTTVDFAAATTMSYNVPNAKIWGWDVMTKYTTDLFSLDVAYNRTRGKDTDTGEYISSINPDTVTSTLNIPIAHSGFSVGWVGTFADRSTHISSSYSKQPGYGVNDFYVSYQGQQALKGMTTTLVLGNAFDKEYWSPQGIPQDGRNGKIFVSYQWW

SEQ ID NO: 54 - protein, 507 aa, unidentified; identical to SEQ ID NO: 52
  REGION 1..507: note = rCPR; rat P450 reductase

SEQ ID NO: 55 - protein, 749 aa, unidentified
  REGION 1..749: note = pCPR; Psilocybe cyanescens
MTDPNRTTFSSALHPLAVVSMASSSSDVFVLGLGVVLAALYIFRDQLFAASKPKVAPVSTTKPANGSANPRDFIAKMKQGKKRIVIFYGSQTGTAEEYAIRLAKEAKQKFGLASLVCDPEEYDFEKLDQLPEDSIAFFVVATYGEGEPTDNAVQLLQNLQDDSFEFSNGERKLSGLKYVVFGLGNKTYEHYNLIGRTVDAQLAKMGAVRVGERGEGDDDKSMEEDYLEWKDGMWDAFAAAMGVEEGQGGDSADFVVSELESHPPEKVYLGEYSARALTKTKGIHDAKNPLAAPITVARELFQSVVDRNCVHVEFNIEGSGITYQHGDHVGLWPLNPDVEVERLLCVLGLTEKRDAVISIESLDPALAKVPFPVPTTYAAVLRHYIDVSAVAGRQILGTLSKFAPTPEAEAFLKNLNTNKEEYHNVVANGCLKLGEILQVATGNDITVAPTPGNTTKWPIPFDIIVSAIPRLQPRYYSISSSPKVHPNTIHATVVVLKYENVPTDPIPRKWVYGVGSNFLLNLKHAINKEPVPFITQNGEQRVGVPEYLIAGPRGSYKTESHFKAPIHVRRSTFRLPTNPKSPVIMIGPGTGVAPFRGFVQERVALARRSVEKNGPESLNDWGRISLFYGCRRSDEDFLYKDEWPQYQEELKGKFKLHCAFSRENYKPDGSKIYVQDLIWEDREHIADAILNGKGYVYICGEAKSMSKQVEEVLARILGEAKGGSGAVEGVAEIKLLKERSRLMLDYELA

SEQ ID NO: 56 - protein, 746 aa, unidentified
  REGION 1..746: note = aCPR: Agrocybe pediades uncharacterized gene with homology toNADPH cytochrome P450 reductase
MSYSNHSTAFPNPLSAYNMASSSSDVFILGLGIVLAALYIFRDQLFAASKPKVAPVAASKAANGSGNPRDFIAKMKEGKKRIVIFYGSQTGTAEEYAIRLAKEAKQKFGLASLVCDPEEYDFENLDQLPEDCAAFFVMATYGEGEPTDNAVQLMQNLQDESFEFSNGERKLEGLKYVVFGLGNKTYEHYNLIGRNVDAALSKMGAIRIGERGEGDDDKSMEEDYLEWKDGMWEAFATAMNVEEGQGGDSADFVVSELESHPEDKVYLGEYSARALTRTKGIHDAKNPYAAPIVVARELFSANADRNCVHIEFNTEGSGITYQHGDHVGLWPLNPDVEVERLLCVLGLTQKKDTVIGIESLDPALAKVPFPVPTTYGTVLRHYIDISAVAGRQILGTMSKFAPTPEAEAFLKNLNTNKEEYHRVVHDGCLKLGEVLQLAAGNNIKAIPTSENTTQWKIPFDIIVSAIPRLQPRYYSISSSPKLHPNSIHATAVVLKYQNVPSELVTSKWVYGVGSNFLLSLKHASNNEPIPVISENGEERVAVPAYLIEGPRGSYKTETHYKAPIHVRRSTFRLPTNPKSPVIMVGPGTGVAPFRGFVQERVALARRSIEKNGPDALNDWGRISLFYGCRREDEDFLYKDEWAKYEEELKGKFKMHCAFSRQNYKPDGSKIYVQDLIWEDREHIADAILNGKGYVYICGEAKSMSKQVEEVLARILGEAKGGSAEVEGAAEIKLLKERSRLMLDVWS

SEQ ID NO: 57 - protein, 368 aa, unidentified
  REGION 1..368: note = malE'
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTV

(19) United States
(12) Patent Application Publication
(10) Pub. No.: US 2025/0171815 A1
(43) Pub. Date: May 29, 2025

(54) PRODUCTION OF PSYCHEDELIC COMPOUNDS

(71) Applicant: CORE ONE LABS INC., Vancouver (CA)

(72) Inventor: Jan BURIAN, Victoria (CA)

(21) Appl. No.: 18/727,672

(22) PCT Filed: Jan. 9, 2023
(86) PCT No.: PCT/CA2023/050016

§ 371 Date: Jul. 9, 2024

Related U.S. Application Data
(60) Provisional application No. 63/297,867, filed on Jan. 10, 2022.

(51) Int. Cl. C12P 17/10; A61K 31/4045; A61K 31/675; C12N 9/02; C12N 9/10; C12N 9/24; C12N 9/88; C12N 15/52; C12N 15/70

(52) U.S. Cl. CPC C12P 17/10; A61K 31/4045; A61K 31/675; C12N 9/0036; C12N 9/0042; C12N 9/0071; C12N 9/1007; C12N 9/1085; C12N 9/2497; C12N 9/88; C12N 15/52; C12N 15/70; C12Y 106/01001; C12Y 106/02004; C12Y 401/01028; C12Y 401/99001; C12N 2830/002

(57) ABSTRACT

This disclosure relates to recombinant microbes, systems, and methods useful in the production of psilocybin and dimethyltryptarme (DMT). The disclosure relates to modifications and optimizations to genes and enzymes directly involved in the psilocybin and DMT pathway for the production of psilocybin and DMT in a host cell. The disclosure also relates to modifications and optimizations to genes and enzymes in the host cell and medium for psilocybin and DMT production in E. coli. The present disclosure also relates to a semi-synthetic method of producing psilocybin.

### FIELD

This disclosure relates to the field of production of psychedelic compounds. More particularly, this disclosure relates to recombinant microbes, systems, and methods useful in the production of psilocybin, dimethyltryptamine (DMT), and intermediates thereof.

### BACKGROUND

Several studies have shown the benefits and efficacy in the use of psilocybin and/or dimethyltryptamine (DMT) for safe and effective treatment and management of various mental health conditions and addiction. Psilocybin was first isolated from the Central American mushroom Psilocybe Mexicana in 1957, and synthetic psilocybin was created shortly thereafter and continues to be used today. Enzymes involved in the production of psilocybin, dimethyltryptamine (DMT), and intermediates thereof are derived originally from fungal genes found in various mushroom varieties and recombinant production in fungi and other eukaryotic host cells has been attempted with varying levels of success.

While Escherichia coli (E. coli) cells provide a convenient and efficient means for recombinant protein production, it has been a challenge to re-create the fungal-based system in E. coli. Thus far, production of psilocybin, DMT, and intermediates within these pathways in E. coli has been characterized by low yields, contamination and often prohibitively expensive end products.

### SUMMARY

The present disclosure relates to modifications and optimizations to genes and enzymes directly involved in the psilocybin and DMT pathway for the production of psilocybin and DMT in a host cell. The present disclosure also relates to modifications and optimizations to genes and enzymes in the host cell and medium for psilocybin and DMT production in E. coli. The present disclosure also relates to a semi-synthetic method of producing psilocybin.

Various aspects of the disclosure relate to a recombinant microbial cell comprising a biosynthetic pathway for producing psilocybin, or intermediates thereof, the microbial cell comprising a heterologous nucleic acid encoding one or more psilocybin production genes. The one or more psilocybin production genes is a tryptophan decarboxylase, a phosphotransferase, a methyltransferase, a monooxygenase, or a combination thereof. The nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 1 to 3. The nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 20 to 22. The nucleic acid sequence encoding the phosphotransferase comprises a sequence having at least 70%, at least 80%, at least 90%, at least 95%, at least 96%, at least 97%, at least 98%, at least 99%, or 100% sequence identity to SEQ ID NO: 4 or 5. The nucleic acid sequence encoding the phosphotransferase comprises a sequence having at least 70%, at least 80%, at least 90%, at least 95%, at least 96%, at least 97%, at least 98%, at least 99%, or 100% sequence identity to SEQ ID NO: 23 or 24. The nucleic acid sequence encoding the methyltransferase comprises a sequence having at least 70%, at least 80%, at least 90%, at least 95%, at least 96%, at least 97%, at least 98%, or at least 99% sequence identity to SEQ ID NO: 6 or 7. The nucleic acid sequence encoding the methyltransferase comprises a sequence having at least 70%, at least 80%, at least 90%, at least 95%, at least 96%, at least 97%, at least 98%, or at least 99%, or at least 100% sequence identity to SEQ ID NO: 25 or 26. The nucleic acid sequence encoding the monooxygenase comprises a sequence having at least 70%, at least 80%, at least 90%, at least 95%, at least 96%, at least 97%, at least 98%, at least 99%, or 100% sequence identity to SEQ ID NO: 14. The nucleic acid sequence encoding the monooxygenase comprises a sequence having at least 70%, at least 80%, at least 90%, at least 95%, at least 96%, at least 97%, at least 98%, at least 99%, or 100% sequence identity to SEQ ID NO: 33. The nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 2, 4, and 6. The nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 21, 23, and 25. The nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 2, 4, and 7. The nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 21, 23, and 26. The nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 24. The heterologous nucleic acid further comprises a NADPH-cytochrome P450 reductase (CPR) gene. The CPR gene comprises a sequence having at least 70%, at least 80%, at least 90%, at least 95%, at least 96%, at least 97%, at least 98%, at least 99%, or 100% sequence identity to SEQ ID NO: 16, 17, or 18.

Various aspects of the disclosure also relate to a system for producing psilocybin comprising a bioreactor comprising a growth medium, and the recombinant microbial cell as defined herein. The microbial cell is cultured with one or more intermediates of psilocybin. The one or more intermediates of psilocybin is psilocin and psilocin is produced by chemical synthesis.

Various aspects of the disclosure also relate to a system for producing psilocybin comprising a bioreactor comprising a growth medium, the recombinant microbial cell as defined herein, and arabinose, wherein arabinose induces expression of the one or more psilocybin producing genes.

Various aspects of the disclosure also relate to a system for producing psilocybin comprising a bioreactor comprising a growth medium, the recombinant microbial cell as defined in any one of claims 1 to 53 and 57 to 59, glucose, and IPTG, wherein IPTG induces expression of the one or more psilocybin producing genes.

Various aspects of the disclosure also relate to a method of producing psilocybin comprising culturing the recombinant microbial cell as defined herein in a growth medium.

D Various aspects of the disclosure also relate to a method for producing psilocybin comprising culturing the recombinant microbial cell as defined herein in a growth medium and adding arabinose to the growth medium to induce expression of the one or more psilocybin producing genes.

Various aspects of the disclosure also relate a method for producing psilocybin comprising culturing the recombinant microbial cell as defined herein in a growth medium comprising glucose and adding IPTG to induce expression of the one or more psilocybin producing genes.

Various aspects of the disclosure also relate to a nucleic acid molecule comprising a sequence having at least 70%, at least 80%, at least 90%, at least 95%, at least 96%, at least 97%, at least 98%, at least 99%, or 100% sequence identity to any one of SEQ ID NOs: 1 to 7, 13 to 18, 20 to 26, and 32 to 37, or a combination thereof.

Various aspects of the disclosure also relate to an expression vector for producing psilocybin in a microbial cell, the expression vector comprising the nucleic acid molecule as defined herein.

Various aspects of the disclosure also relate to psilocybin produced from the method as defined herein for treating a mental health condition.

Various aspects of the disclosure also relate to a pharmaceutical composition comprising psilocybin produced from the method as defined herein and a pharmaceutically acceptable carrier for treating a mental health condition.

Various aspects of the disclosure also relate to use of psilocybin produced from the method as defined herein for treating a mental health condition.

Various aspects of the disclosure also relate to use of psilocybin produced from the method as defined herein in preparation of a medicament for treating a mental health condition.

Various aspects of the disclosure also relate to use of a pharmaceutical composition comprising psilocybin produced from the method as defined herein and a pharmaceutically acceptable carrier for treating a mental health condition.

Various aspects of the disclosure also relate to use of a pharmaceutical composition comprising psilocybin produced from the method as defined herein and a pharmaceutically acceptable carrier in preparation of a medicament for treating a mental health condition.

Various aspects of the disclosure also relate to a recombinant microbial cell comprising a biosynthetic pathway for producing dimethyltryptamine (DMT), or intermediates thereof, the microbial cell comprising a heterologous nucleic acid encoding one or more DMT production genes. The one or more DMT production genes is a tryptophan decarboxylase, an indolethylamine N-methyltransferase (INMT), or a combination thereof. The nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 1 to 3. The nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 20 to 22. The INMT comprises a sequence having at least 70%, at least 80%, at least 90%, at least 95%, at least 96%, at least 97%, at least 98%, at least 99%, or 100% sequence identity to any one of SEQ ID NOs: 8 to 12. The INMT comprises a sequence having at least 70%, at least 80%, at least 90%, at least 95%, at least 96%, at least 97%, at least 98%, at least 99%, or 100% sequence identity to any one of SEQ ID NOs: 27 to 31.

Various aspects of the disclosure also relate to a system for producing dimethyltryptamine (DMT) comprising a bioreactor comprising a growth medium, and the recombinant microbial cell as defined herein.

Various aspects of the disclosure also relate to a method of producing dimethyltryptamine (DMT) comprising culturing the recombinant microbial cell as defined herein in a growth medium to produce DMT.

Various aspects of the disclosure also relate to a nucleic acid molecule comprising a sequence having at least 70%, at least 80%, at least 90%, at least 95%, at least 96%, at least 97%, at least 98%, at least 99%, or 100% sequence identity to any one of SEQ ID NOs: 1 to 3, 8 to 13, 20 to 22, and 27 to 32, or a combination thereof.

Various aspects of the disclosure also relate to an expression vector for producing dimethyltryptamine (DMT) in a microbial cell, the expression vector comprising the nucleic acid molecule as defined herein.

Various aspects of the disclosure also relate to dimethyltryptamine (DMT) produced from the method as defined herein for treating a mental health condition.

Various aspects of the disclosure also relate to a pharmaceutical composition comprising dimethyltryptamine (DMT) produced from the method as defined herein and a pharmaceutically acceptable carrier for treating a mental health condition.

Various aspects of the disclosure also relate to use of dimethyltryptamine (DMT) produced from the method as defined herein for treating a mental health condition.

Various aspects of the disclosure also relate to use of dimethyltryptamine (DMT) produced from the method as defined herein in preparation of a medicament for treating a mental health condition.

Various aspects of the disclosure also relate to use of a pharmaceutical composition comprising dimethyltryptamine (DMT) produced from the method as defined herein and a pharmaceutically acceptable carrier for treating a mental health condition.

Various aspects of the disclosure also relate to use of a pharmaceutical composition comprising dimethyltryptamine (DMT) produced from the method as defined herein and a pharmaceutically acceptable carrier in preparation of a medicament for treating a mental health condition.

Other aspects and features of the present invention will become apparent to those ordinarily skilled in the art upon review of the following description of specific embodiments of the invention

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a diagram showing the recombinant pathways constructed in Escherichia coli (E. coli) leading to synthesis of psilocybin or N,N-dimethyltryptamine (DMT) starting from L-tryptophan, 4-hydroxyindol and psilocin.

FIGS. 2A and 2B are images showing expression of L-tryptophan and 4-hydroxytryptophan decarboxylases. A. Western blot developed with anti-his tag polyclonal serum. B. Coomassie blue stained SDS PAGE gel. Lane 1—RumD (MW 55.92K), Lane 2—CloD (MW 49.37K), Lane 3—PsiD_PCU (MW 50.61K), Lane 4—uninduced control. E. coli TOP10 was cultivated in Terrific Broth (TFB) with shaking at 37° C. overnight. Production of enzymes was induced by addition of 0.15% L-arabinose. The arrow points to the RumD band.

FIGS. 3A to 3F are graphs showing results of HPLC analysis of decarboxylation of L-tryptophan to tryptamine. E. coli BL21 harbored plasmids expressing relevant decarboxylases. Production of enzymes was induced by addition of 0.15% L-arabinose. L-tryptophan and Tryptamine peaks were eluted at 14 & 15 min. respectively. A. Chromatogram of TFB. L-tryptophan peak represents its content in TFB; B. Chromatogram of TFB after 4 hours of fermentation by BL21(pBAD-DCLO) expressing cloD from C. sporogenes; C. Chromatogram of TFB after 4 hours of fermentation by BL21(pBAD-DRUM) expressing rumD from R. gnavus; D. Chromatogram of TFB after 19 hours of fermentation by BL21(pBAD-DCLO) expressing cloD from C. sporogenes; E. Chromatogram of TFB after 19 hours of fermentation by BL21(pBAD-DRUM) expressing rumD from R. gnavus; F. Chromatogram of TFB after 19 hours of fermentation by BL21(pBAD-psiD_CU) expressing psiD from P. cubensis.

FIGS. 4A to 4C are graphs showing results of HPLC analysis of conversion (decarboxylation) of 4-hydroxytryptophan to 4-hydroxytryptamine. 4-Hydroxytryptophan and 4-Hydroxytryptamine peaks were eluted at 10.2 & 9.7 min. respectively. A. Chromatogram of MAM-lac containing 4-hydroxytryptophan (MAM-lac-4htrp); B. Chromatogram of media MAM-lac-4htrp after 19 hours of fermentation by BL21(pBAD-DRUM) expressing rumD from R. gnavus; C. Chromatogram of media MAM-lac-4htrp after 19 hours of fermentation by BL21(pBAD-DCLO) expressing cloD from C. sporogenes.

FIGS. 5A and 5B are graphs showing results of HPLC analysis of synthesis of 4-hydroxytryptophan by E. coli BL21. 4-hydroxyindole and 4-hydroxytryptophan peaks were eluted at 12 & 10.2 min. respectively. A. Chromatogram of MAM-lac containing 4-hydroxyindole (MAM-lac-4hi); B. Chromatogram of media MAM-lac-4hi after 19 hours of fermentation by E. coli BL21.

FIGS. 6A and 6B are images showing expression of E. coli TOP10 whole cell lysates expressing 4-hydroxytryptamine/psilocin kinases. A. Western blot developed with anti-his tag polyclonal serum. B. Coomassie blue stained SDS PAGE gel. Lane 1—PsiK_PCY (MW 41.27K), Lane 2—PsiK_PCU (MW 41.40K), Lane 3—uninduced control.

FIGS. 7A to 7C are graphs showing results of HPLC analysis of conversion (phosphorylation) of 4-hydroxytryptamine to norbaeocystin. 4-hydroxytryptamine and norbaeocystin peaks were eluted at 9.7 & 3.7 min. respectively. A. Chromatogram of MAM-lac containing 4-hydroxytryptamine (MAM-lac-4htry); B. Chromatogram of MAM-lac-4htry after 19 hours of fermentation by BL21(pBAD-KPCU) expressing kinase PsiK_PCU from P. cubensis. C. Chromatogram of MAM-lac-4htry after 19 hours of fermentation by BL21(pBAD-KPCY) expressing kinase PsiK_PCY from P. cyanescens.

FIGS. 8A and 8B are images showing expression of selected indolethylamine N-methyltransferases (INMTs). A. Western blot developed with anti-his tag polyclonal serum. B. Coomassie blue stained SDS PAGE gel. Lane 1—rINMT-h (MW 29.22K) Lane 2—rINMT (MW 30.04K), Lane 3—fINMT (MW 30.78K), Lane 4—sINMT (MW 31.25K), Lane 5—wINMT (MW 25.93K), Lane 6—uninduced control.

FIG. 8C is a graph showing results of HPLC analysis of methylation of tryptamine to produce monomethyl tryptamine (MMT). MAM media was supplemented with sorbitol and tryptamine after fermentation with E. coli TOP10(pBAD24E-rINMT-h, p15A-MTNN) with shaking at 37° C. The expression of rINMT-h and MTNN was induced by addition of 0.015% L-arabinose. The large peak is tryptamine and the small peak is MMT (the inset window showing an enlarged portion of the spectrum). MMT was verified by its UV spectrum.

FIGS. 9A and 9B are images showing co-expression of methylases and mtnN. SDS PAGE analysis of E. coli TOP10 whole cell lysates expressing psiM methylases (*) and mtnN (4). A. Western blot developed with anti-his tag polyclonal serum. B. Coomassie blue stained SDS PAGE gel. Lane 1—uninduced control, Lane 2—PsiM_PCU and MTNN, Lane 3—PsiM_PCY and MTNN.

FIG. 10A is a chart showing blastn results of rCPR against the P. cyanescens genome: hypothetical protein CVT25_015047.

FIG. 10B is a chart showing GenBank Protein Database search results for homologous protein with hypothetical protein CVT25_015047 from P. cyanescens.

FIGS. 11A and 11B are images showing expression of NADPH-cytochrome P450 reductases (CPRs). SDS PAGE analysis of E. coli TOP10 whole cell lysates with cloned CPRs. A. Western blot developed with anti-his tag polyclonal serum. B. Coomassie blue stained SDS PAGE gel. Lane 1—rCPR (MW 77.92K), Lane 2—aCPR (MW 83.74K), Lane 3—pCPR (MW 83.74K), Lane 4—MalE′ (MW 41.4K). Only aCPR and MalE′ proteins were detected by SDS PAGE gel and corresponding Western blot. Proteins rCPR and pCPR were not detected. MalE′ protein, lacking its signal sequence, was developed as a positive control for L-arabinose induction and Western blot procedure.

FIG. 12 is a diagram showing a method for chemical synthesis of psilocin.

FIG. 13A to 13C are graphs showing results of HPLC analysis of conversion (phosphorylation) of psilocin to psilocybin. Psilocin and psilocybin peaks were eluted at 5.3 & 11.5 min. respectively. A. Chromatogram of MAM-lac containing Psilocin (MAM-lac-psl); B. Chromatogram of MAM-lac-psl after 19 hours of fermentation by BL21(pBAD-KPCU) expressing kinase PsiK_PCU from P. cubensis; C. Chromatogram of MAM-lac-4htry after 19 hours of fermentation by BL21(pBAD-KPCY) expressing kinase PsiK_PCY from P. cyanescens.

FIG. 14 is diagram of plasmid pBAD24E, a modified plasmid pBAD24 created by insertion of EcoRI adapter into NsiI site. Expression is fully induced by addition of 0.2% arabinose and relatively highly repressed in the presence of glucose.

FIG. 15 is a diagram of plasmid pUC19-tac1 created by ligating a synthetic DNA fragment encoding for lacl^{q}, promoter tac and multiple cloning site between EcoRI and HindIII restriction sites of plasmid pUC19, resulting in a high copy number plasmid.

FIG. 16 is a diagram of plasmid p15A-Km, a synthetic plasmid with p15A origin of replication, multiple cloning site from pUC18 and optimized kanamycin resistance gene that has relatively low copy number (˜15).

FIG. 17 is a diagram of plasmid pCM-BH created by ligation of two PCR products: a fragment with chloramphenicol resistance gene cat (823 bp) and a 1341 bp fragment with pUC type origin of replication resulting in a high copy plasmid (˜600).

FIG. 18 is a diagram of plasmid pKLPR-N, a synthetic plasmid with a kanamycin selection marker based on the pKL1 replicon with copy number higher than pBAD24E. The plasmid was designed to induce expression with temperature shift from lambda phage promoter controlled by the C1875 thermosensitive repressor.

FIG. 19 is a diagram of plasmid pBAD24E-MyKyDr according to an embodiment of the invention.

FIG. 20 is a diagram of plasmid pBAD24E-DrKyMy according to an embodiment of the invention.

FIG. 21 is a diagram of plasmid pBAD24E-KyDrMu according to an embodiment of the invention.

FIG. 22 is a diagram of plasmid pBAD24E-DrMyKy according to an embodiment of the invention.

FIG. 23 is a diagram of plasmid pBAD24E-KyDrMy according to an embodiment of the invention.

FIG. 24 is a diagram of plasmid pBAD24E-DrKyMu according to an embodiment of the invention.

FIG. 25 is a graph showing results of HPLC analysis of conversion of 4-hydroxytryptophan to psilocybin by E. coli BL21-AtnaA-AaraA (pBAD24E-DrKyMu). Conversion was induced by 0.15% arabinose. Measured concentration of psilocybin was 96.7 μg/ml. Measured concentration of Norbaeocystin and Baeocystin was 36.6 μg/ml and 49.9 μg/ml respectively. The peak eluted at 12.78 min was Tryptamine.

FIG. 26 is a graph showing results of HPLC analysis of conversion of 4-hydroxytryptophan to psilocybin by E. coli BL21-DtnaA-DaraA (pBAD24E-DrKyMu). Conversion was induced by 0.015% arabinose. Measured concentration of psilocybin was 60.6 mg/ml. Measured concentration of Norbaeocystin and Baeocystin was 42.6 mg/ml and 51.5 mg/ml respectively. The peak eluted at 12.79 min was Tryptamine.

FIG. 27 is a diagram of plasmid pKLBAD-DrKyMu according to an embodiment of the invention.

FIG. 28 is a diagram of plasmid pKLBAD-KyDrMy according to an embodiment of the invention.

FIG. 29 is a diagram of plasmid pBAD15A-DrKyMu according to an embodiment of the invention.

FIG. 30 is a diagram of plasmid pBAD15A-KyDrMy according to an embodiment of the invention.

FIGS. 31A to 31D are graphs showing results of HPLC analysis of conversion of 4-hydroxytryptophan to psilocybin by E. coli BL21-AtnaA-AaraA (pKLBAD-DrKyMu). Expression of enzymes was A. constitutive in MAM-lactose media, or induced by addition of B. 0.0015%, C. 0.015% and D. 0.15% arabinose. Psilocybin was eluted as double peak (confirmed by UV spectra) at 4.8-4.93 min. Measured concentration of psilocybin was 82.8 μg/ml (A), 49.3 μg/ml (B), 14.9 μg/ml (C) and 18.6 μg/ml (D).

FIG. 32 is a diagram of plasmid pUCtac1-DrKyMu according to an embodiment of the invention.

FIG. 33 is a diagram of plasmid pUCtac1-KyDrMy according to an embodiment of the invention.

FIG. 34 is a diagram of plasmid p15Alqtac1-DrKyMu according to an embodiment of the invention.

FIG. 35 is a diagram of plasmid p15Alqtac1-KyDrMy according to an embodiment of the invention.

FIG. 36 is a diagram of plasmid pKLPR-KyDrMy according to an embodiment of the invention.

FIG. 37 is a diagram of plasmid pKLPR-DrMyKy according to an embodiment of the invention.

FIGS. 38A to 38D are graphs showing results of HPLC analysis of conversion of 4-hydroxytryptophan to psilocybin by E. coli BL21-ΔtnaA-ΔaraA (p15Alqtac1-DrKyMu). Expression of enzymes was A. constitutive in MAM-2%-glucose media, or induced by addition of B. 0.001 mM, C. 0.01 mM, and D. 1 mM IPTG. Psilocybin was eluted at 5.67 min. Measured concentration of psilocybin was 68.5 μg/mL (A), 65.4 μg/mL (B), 103.9 μg/ml (C) and 47.8 μg/mL (D).

FIG. 39 is an image showing a Western blot of BL21-ΔtnaA-ΔaraA(p15Alqtac1-DrKyMu). Cultivation was carried out in MAM-2%-glucose with 150 μg/mL of 4-hydroxyindole. Molecular weight protein standards (MW); host strain BL21-ΔtnaA-ΔaraA (1); BL21-ΔtnaA-ΔaraA(p15Alqtac1-DrKyMu) no IPTG (2), 0.001 mM IPTG (3), 0.01 mM IPTG (4) and 1 mM IPTG (5). Control sample (6) with expressed decarboxylase Dr, kinase Ky and methylase Mu.

FIG. 40 is a diagram of plasmid pUCtac1-Ky according to an embodiment of the invention.

FIG. 41 is a diagram of plasmid pUCtac1-Ku according to an embodiment of the invention.

FIG. 42 is a diagram of plasmid p15Alqtac1-Ky according to an embodiment of the invention.

FIG. 43 is a diagram of plasmid p15Alqtac1-Ku according to an embodiment of the invention.

FIGS. 44A to 44C are graphs showing results of HPLC analysis of conversion (phosphorylation) of psilocin (psi) to psilocybin. Psilocin and psilocybin peaks were eluted at 5.11 & 10.45 min. respectively. A. Chromatogram of MAM-glucose containing Psilocin (MAM-glucose-psl); B. Chromatogram of MAM-glucose-psl after 19 hours of fermentation by BL21(pUCtac1-Ky) expressing kinase PsiK_PCY from P. cyanescens; C. Chromatogram of MAM-glucose-psl after 19 hours of fermentation by BL21(pUCtac1-Ku) expressing kinase PsiK_PCU from P. cubensis.

FIGS. 45A to 45C are graphs showing results of HPLC analysis of conversion (phosphorylation) of Psilocin (psi) to psilocybin. Psilocin and psilocybin peaks were eluted at 5.11 & 10.45 min. respectively. A. Chromatogram of MAM-glucose (glu) containing Psilocin (MAM-glu-psl); B. Chromatogram of MAM-glu-psl after 19 hours of fermentation by BL21(p15Al^{q}tac1-Ky) expressing kinase PsiK_PCY from P. cyanescens; C. Chromatogram of MAM-glu-psl after 19 hours of fermentation by BL21(p15Al^{q}tac1-Ku) expressing kinase PsiK_PCU from P. cubensis.

### DETAILED DESCRIPTION

### Definitions

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

Articles such as “a” and “an” when used in a claim, are understood to mean one or more of what is claimed or described.

Terms of degree such as “about”, “approximately” and “substantially” as used herein mean a reasonable amount of deviation of the modified term such that the end result is not significantly changed. These terms may refer to a measurable value such as an amount, a temporal duration, and the like, and are meant to encompass variations of +/−0.1% of the given value, +/−0.5%, +/−1%, +/−2%, +/−5% or +/−10%.

The term “bioreactor” as used herein refers to a vessel used for the growth of microorganisms in a growth medium or culture. A bioreactor can be of any size so long as it is useful for culturing cells. Internal conditions of a bioreactor, including, but not limited to pH, osmolarity, C02 saturation, 02 saturation, temperature and combinations thereof, are typically controlled during the culturing period. A bioreactor can be composed of any material that is suitable for holding cells in media under the culture conditions described herein, including glass, plastic or metal. One of ordinary skill in the art will be aware of and will be able to choose suitable bioreactors for use in practicing the present invention. As used herein, the term “bioreactor” and “fermentor” may be used interchangeably.

The terms “comprises”, “comprising”, “include”, “includes”, “including”, “contain”, “contains” and “containing” as used herein are meant to be non-limiting, i.e., other steps and other sections which do not affect the end result can be added. The above terms encompass the terms “consisting of” and “consisting essentially of”.

“Decreasing expression”, “decreasing activity”, “reducing expression”, and “reducing activity” as used herein are intended to encompass well known equivalent terms regarding expression and activity such as “inhibiting”, “down-regulating”, “knocking out”, “silencing”, etc.

An “endogenous” gene as referred to herein refers to a gene that is naturally present in an organism (e.g., a prokaryotic cell) and has not been introduced through genetic modification. An “endogenous” gene may be distinguished from a second copy of the gene that is introduced by, for example, genetic modification, and exists at a separate locus in the genome.

“Expression” or “expressing” as used herein refers to the process by which information from a gene is used in the synthesis of a functional gene product, and may relate to production of any detectable level of a product, or activity of a product, encoded by a gene. Gene expression may be modulated (i.e., initiated, increased, decreased, terminated, maintained, or precluded) at many levels including transcription, RNA processing, translation, post-translational modification, protein degradation. In the context of the present disclosure, reduced expression of an endogenous gene can be affected by reduced transcription of the endogenous gene, by reduced translation of mRNA transcripts of the endogenous gene, or by the introduction of mutations that either prevent the translation of functional polypeptides or result in the translation of polypeptides with reduced abilities to convert substrate. Such reduced expression of the endogenous gene may result from expression of transgenes comprising expression constructs designed to reduce expression of the endogenous genes.

“Expression construct” as used herein refers to any type of genetic construct containing a nucleic acid coding for a gene product in which part or all the nucleic acid encoding sequence is capable of being transcribed. The transcript may be translated into a protein, but it need not be. An expression construct of the disclosed nucleic acid molecule may further comprise a promoter and other regulatory elements, for example, an enhancer, a silencer, a polyadenylation site, a transcription terminator, a selectable marker or a screenable marker.

A “gene” as used herein refers to a nucleic acid molecule or a portion thereof, the sequence of which includes information required for the production of a particular protein or polypeptide chain. The polypeptide can be encoded by a full-length sequence or any portion of the coding sequence, so long as the functional activity of the protein is retained. A “heterologous” region of a nucleic acid construct (i.e., a heterologous gene) is an identifiable segment of DNA within a larger nucleic acid construct that is not found in association with the other genetic components of the construct in nature not present in the natural host.

A “genetic modification” as used herein broadly refers to any novel combination of genetic material obtained with techniques of modern biotechnology. Genetic modifications include, but are not limited to, “transgenes” in which the genetic material has been altered by the insertion of exogenous genetic material. However, genetic modifications also include alterations (e.g., insertions, deletions, or substitutions) in endogenous genes introduced in a targeted manner with techniques such as CRISPR/Cas9, TALENS, etc. as discussed elsewhere herein. Genetic modifications may be transient or stably inherited.

“Heterologous” or “exogenous” as used herein refers to DNA that does not occur naturally as part of the host organism's genome or is not normally found in the host genome in an identical context.

The term “medium”, “growth medium”, or “cell culture medium”, as used herein refer to a solution containing nutrients which nourish growing cells. Typically, these solutions may provide essential and nonessential amino acids, vitamins, energy sources, lipids, and trace elements required by the cell for minimal growth and/or survival. The solution may also contain components that enhance growth and/or survival above the minimal rate, including growth factors. In some embodiments, the medium may also comprise one or more antibiotics, which serve as selectable markers to ensure that virtually all cells retain the plasmid which encodes the target protein. In some embodiments, medium is formulated to a pH and salt concentration optimal for cell survival and proliferation.

As used herein, the term “nucleic acid”, “nucleic acid molecule”, or “polynucleotide(s)” refers to RNA, such as mRNA, or in the form of DNA, including, for instance, cDNA and genomic DNA obtained by cloning or produced by chemical synthesis or by a combination thereof. The polynucleotides may be recombinant polynucleotides. The DNA may be double-stranded or single-stranded. Single-stranded polynucleotides may be the coding strand, also known as the sense strand, or it may be the non-coding strand, also referred to as the anti-sense strand. Polynucleotides generally refers to any polyribonucleotide or polydeoxribonucleotide, which may be unmodified RNA or DNA or modified RNA or DNA. In addition, polynucleotide as used herein refers to triple-stranded regions comprising RNA or DNA or both RNA and DNA. The strands in such regions may be from the same molecule or from different molecules. The regions may include all of one or more of the molecules of a triple-helical region often is an oligonucleotide. Moreover, DNA or DNA comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those skilled in the art. The term “polynucleotide” as it is used herein embraces such chemically, enzymatically or metabolically modified forms of polynucleotides, as well as the chemical forms of DNA and RNA characteristics of viruses and cells.

“Promoter” as used herein refers to a nucleotide sequence that directs the initiation and rate of transcription of a coding sequence. Alternatively, constitutive or inducible promoters useful under the appropriate conditions to direct high-level expression of the introduced expression construct could be used.

The term “recombinant” as used herein means recombined or new combinations of nucleic acid sequences, genes, or fragments thereof which are produced by recombinant DNA techniques and are distinct from a naturally occurring nucleic acid sequence.

The term “transformation” as used herein refers to a process whereby exogenous or heterologous DNA (i.e., a nucleic acid construct) is introduced into a recipient host cell (e.g., prokaryotic cells). Therefore, in host cells, the acquisition of exogenous DNA into a host cell is referred to as transformation. With host cells, a stably transformed bacterial cell is one in which the introduced DNA has become integrated into a chromosome so that it is inherited by daughter cells through chromosome replication. This stability is demonstrated by the ability of the host cell to establish cell lines or clones comprised of a population of daughter cells containing the introduced DNA.

The term “vector” as used herein refers to a plasmid or phage DNA or other DNA sequence into which DNA can be inserted to be cloned. The vector can replicate autonomously in a host cell and can be further characterized by one or a small number of endonuclease recognition sites at which such DNA sequences can be cut in a determinable fashion and into which DNA can be inserted. The vector can further contain a marker suitable for use in the identification of cells transformed with the vector. Markers, for example, are tetracycline resistance or ampicillin resistance. The words “cloning vehicle” are sometimes used for “vector”.

### Psilocybin and Dimethyltryptamine (DMT) Pathways and Strains for Production

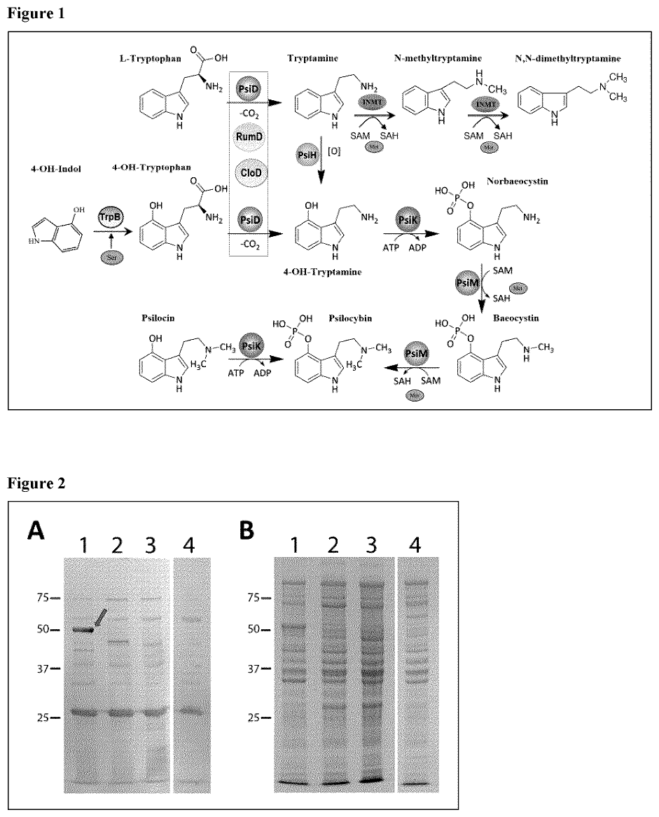

The present inventor redesigned various aspects of the psilocybin and DMT production process for optimized expression and production and managed to recreate the entire production pathway shown in FIG. 1 for use in E. coli. Various technical modifications were introduced to overcome bacteria-specific issues that limited yield and/or purity in other host recombinant systems.

There are four psilocybin biosynthesis enzymes in Psilocybes (FIG. 1). PsiD is a new class of fungal L-tryptophan decarboxylases that synthesizes tryptamine from L-tryptophan. PsiH is a fungal monooxygenase that incorporates a hydroxyl group into tryptamine resulting in 4-hydroxytryptamine. PsiK is a fungal phosphotransferase (kinase) that produces norbaeocystin from 4-hydroxytryptamine. PsiM (S-adenosyl-L-methionine (SAM)-dependent N-methyltransferase) catalyzes N-methyl transfer into norbaeocystin and consequently into baeocystin finally producing psilocybin.

Like psilocybin, DMT (N,N-dimethyltryptamine) can be produced from L-tryptophan. DMT is found in many plants and animals and is both a derivative and a structural analog of tryptamine. It is an analog of psilocybin and has been shown in therapeutic trials to result in significant and very large reductions in retrospective reports of suicidal ideation, cognitive impairment, and symptoms of posttraumatic stress disorder, depression, and anxiety.

The production of DMT from L-tryptophan requires two enzymes, L-tryptophan decarboxylase, and indolethylamine-N-methyltransferase (INMT). Both can be added to the E. coli genome to enable recombinant synthesis. L-tryptophan decarboxylases are found in fungi, mushrooms including genus Psilocybe, plants like genus Actanea (Buttercups), oak, Catharanthus roseus (flowering plant) or in a range of animals from Branchiostoma, small vaguely eel- or snake-like animals, to humans (chromosome 7). Additionally, human gut microflora can convert L-tryptophane to tryptamine.

FIG. 1 presents an overview and representation of pathways that were constructed in E. coli in order to synthesize psilocybin or DMT starting from L-tryptophan, 4-hydroxyindole and psilocin. Thus, both recombinant production methods with a range of cloned enzymes were tested as well as semi-synthesis (providing appropriate precursors to bypass certain reactions). In particular, relevant enzymes were considered from a wide variety of different sources (including other species that do not produce psilocybin or DMT) to construct these pathways for the production of the small molecule intermediates indicated in FIG. 1. All selected enzymes were reverse engineered using selected computer algorithms to provide the sequences of genes optimized for expression in E. coli. DNA from selected gene candidates was synthesized in a gene cassette format to allow construction of selected expression plasmids (and also to allow flexibility in movement from one plasmid to another). Various expression plasmids containing one or more psilocybin or DMT producing genes in different combinations and different operon configurations were tested in order to create functional operons as required for designated production strategies in E. coli.

Cloning and preliminary expression tests were carried out in E. coli TOP10 (F-mcrA Δ(mrr-hsdRMS-mcrBC) ϕb80/acZΔM15 ΔlacX74 recA1 araD139 Δ(araleu)7697 galU galK rpsL (StrR) endA1 nupG), a popular laboratory strain for cloning experiments. Due to deletion of (araleu) this strain would function adequately as a host for L-arabinose-induced expression.

E. coli strain BL21 [F⁻ ompT (Ion) hsdSB (rB⁻ mB⁻) gal dcm] was chosen as a production host. This strain is robust and fast growing in a defined media and number of carbon sources and does not need any cofactors added (e.g., such as thiamine). Induction of an operon encoding a full synthetic pathway, leads to a step-by-step production of all relevant intermediates and finally to the desired molecule (psilocybin or DMT). In order to achieve greater conversion of the starting substrate (L-tryptophan, 4-hydroxyindole or psilocin) for increased yields of psilocybin or DMT, strategies were developed for selection and optimization of pathway genes, and microbial genes, and greater control over the expression processes was employed as discussed below. However, the skilled person will appreciate that other E. coli strains may be substituted for TOP10 and BL21 and still provide the characteristics in a host cell as are described herein.

### Selection and Optimization of Pathway Genes

One or more versions of each of the enzymes in the psilocybin and DMT pathways were tested for expression and catalytic activity in E. coli.

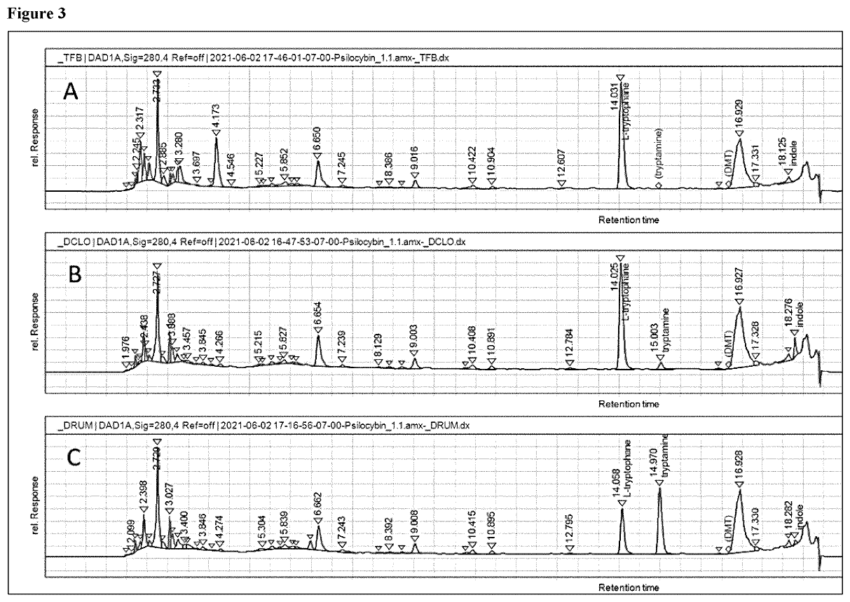

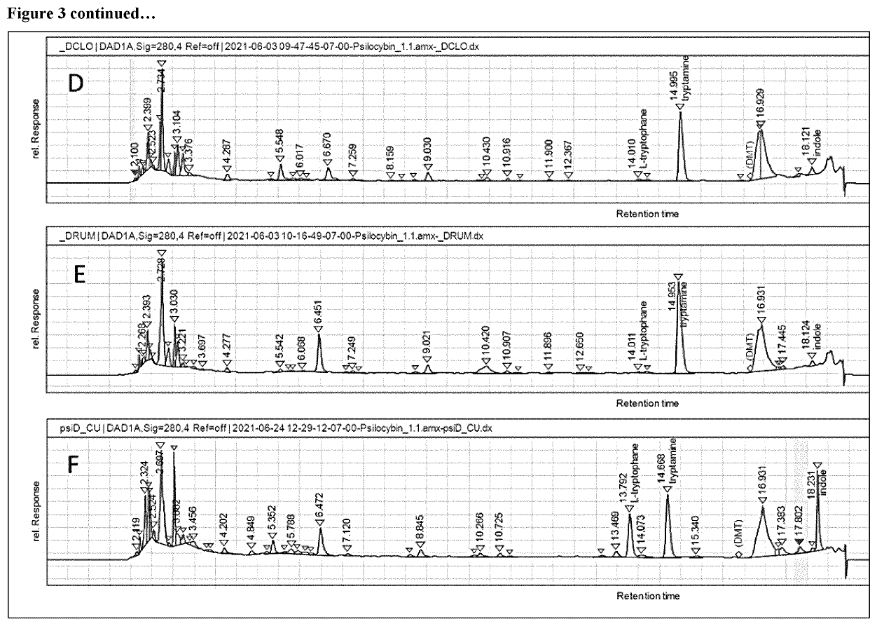

Three recombinant decarboxylases in E. coli were tested and successfully converted L-tryptophan to tryptamine: PsiD_PCU (Psilocibe cubensis) (SEQ ID NO: 1), RumD (Ruminococcus gnavus) (SEQ ID NO: 2) and CloD (Clostridium sporogenes) (SEQ ID NO: 3). Unexpectedly, RumD, which is from the Gram positive anaerobic bacterium Ruminococcus gnavus, a member of the human gut flora, and is phylogenetically very different from Gram negative E. coli, was extremely well expressed (almost 10-fold higher) (FIG. 2A, arrow) and was active in producing tryptamine at 4 hours (FIG. 3).

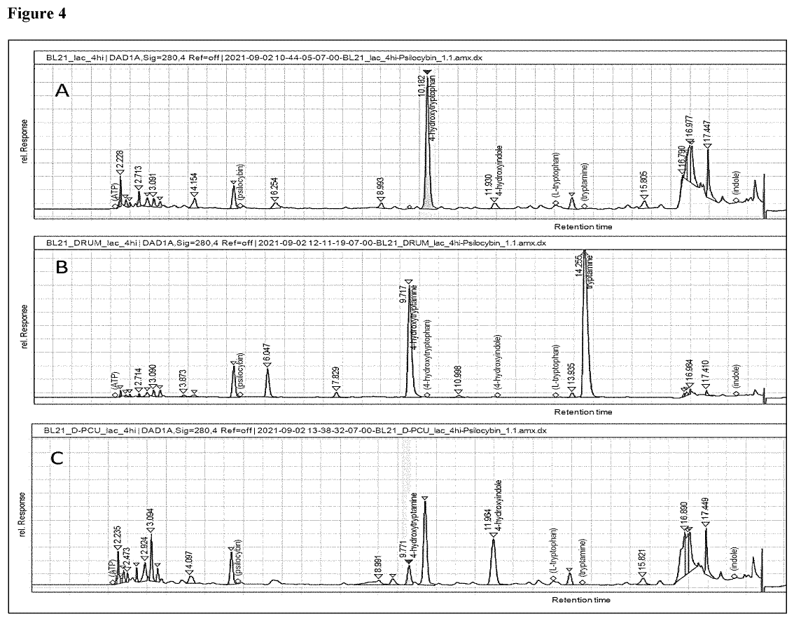

The recombinant decarboxylases PsiD, RumD, and CloD also successfully converted 4-hydroxytryptophan to 4-hydroxytryptamine (FIG. 4). Both RumD and CloD exhibited unexpectedly high levels of enzymatic activity (FIGS. 4B and 4C).

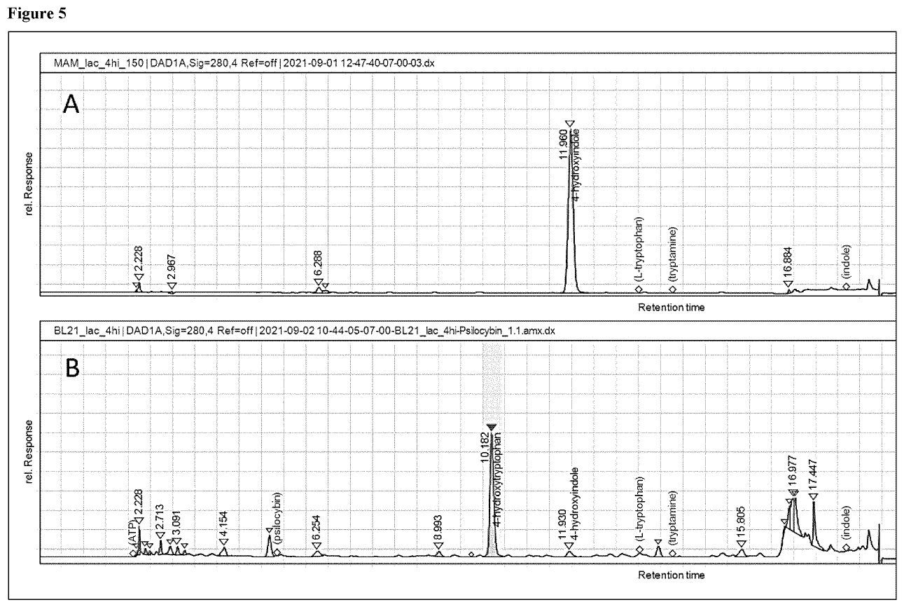

For synthesis of 4-hydroxytryptophan from 4-hydroxyindole and L-serine, it was important to have an adequate source of 4-hydroxytryptophan in cells. Tryptophan synthase complex of E. coli catalyzes the reaction between indole and serine (Ser) to make L-tryptophan (L-Trp). The catalytic subunit beta, TrpB, is known to have wider specificity for indole analogs. E. coli BL21 grown in a minimal medium induces the L-Trp synthesis pathway due to the lack of L-Trp in media and was shown here to synthesize 4-hydroxytryptophan from 4-hydroxyindole and Ser (FIGS. 5A and 5B).

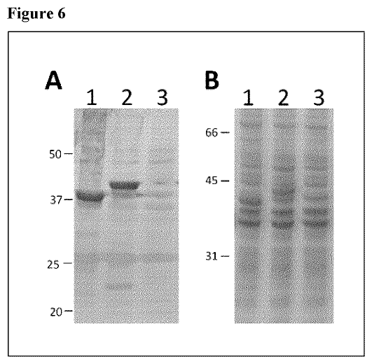

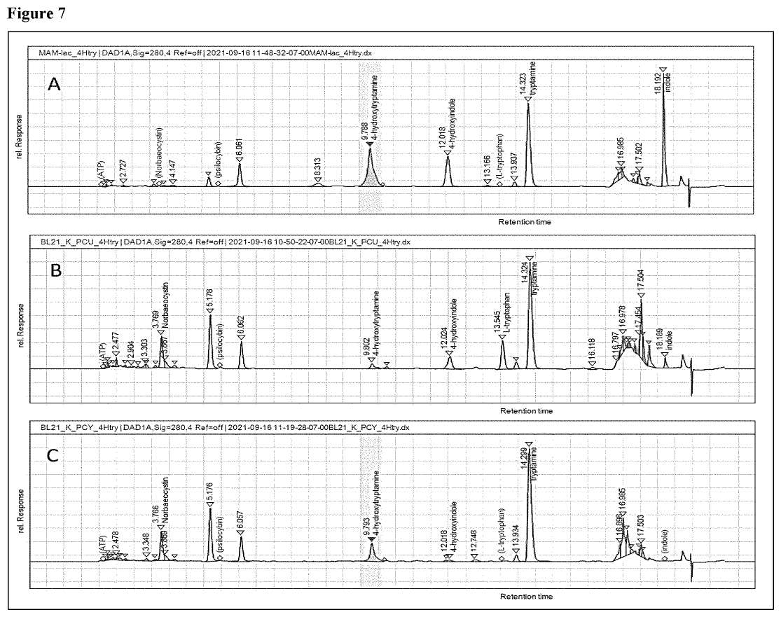

Two kinases, PsiK_PCY (SEQ ID NO: 4) and PsiK_PCU (SEQ ID NO: 5), from P. cyanescens and P. cubensis were very well expressed in E. coli (FIG. 6). Both phosphorylated 4-hydroxytryptamine to produce Norbaeocystin (FIGS. 7A to 7C).

To enable psilocybin synthesis, norbaeocystin methylases PsiM_PCY (SEQ ID NO: 6; Psilocibe cyanescens) and PsiM_PCU (SEQ ID NO: 7; P. cubensis) were expressed in E. coli TOP10 and BL21-ΔtnaA.

Production and use of cytochrome P450 monooxidase, PsiH, in E. coli was more challenging because it requires an NADPH-cytochrome P450 reductase as a redox partner. Due to the challenge associated with production and activity of PsiH in bacterial cells, it has been left out of previous psilocybin production systems utilizing E. coli. PsiH catalyzes the reaction using an oxygen molecule that is reduced to a hydroxyl radical and water by the concomitant oxidation of NAD(P)H resulting in hydroxylation of tryptamine into 4-hydroxytryptamine. Another putative cytochrome P450 reductase was identified from the common fieldcap mushroom Agrocybe pediades as a PsiH_PCY redox partner and this protein is much better expressed in E. coli.

PsiH from Psilocibe cyanescens was selected, designed, and synthesized in an optimized DNA cassette psiH_PCY (SEQ ID NO: 14). Expression of eukaryotic membrane bound P450 monooxidases (hydroxylases), analogous to PsiH, has proven difficult in E. coli. It has been shown, for similar enzymes that the N-terminal amino acids had to be modified or fusion partners were used to achieve expression.

Therefore, the designed cassette contains unique PstI and KpnI restriction sites close to start codon allowing for insertion of XhoI-PstI or XhoI-KpnI DNA fragments enabling a change of N-terminal amino acids to support expression. Also, a library of RBSs was designed with ascending activity and inserted upstream of psiH ORF (XhoI, NsiI insertion) to identify an optimal configuration.

PsiH was not expressed from a simple cloned construct as judged by Western Blot. In most cases, full-length forms of eukaryotic membrane bound P450 genes express as inactive forms or not at all in E. coli cells. In all cases to date, some modification of 5′ nucleotide sequence of P450 genes is important for expression, but this does not always require amino acid changes. In some cases, optimization for E. coli involves choosing the preferred codons, enhancing AT richness, minimizing the potential for secondary structure formation in the mRNA transcript, and substituting the second codon with one found to enhance the expression of a test protein (e.g., using GCT for Ala).

The Pro-Gly rich site is conserved between the N-terminal membrane-anchoring domain and the functional domain of membrane-bound P450 sequences. That region must be preserved. There is a Pro-Gly-rich region in PsiH. The signal sequence, presumably 1-19 amino acids, can be changed or replaced to enable adequate expression. A DNA cassette was designed that allows changes to the N-terminus of PsiH by simple replacement of a short DNA fragment at 5′ end of the psiH gene sequence between NheI and SmaI restriction sites without modification of the PG-rich region.

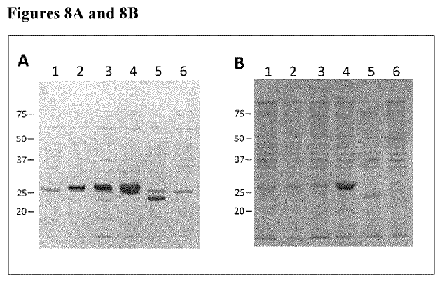

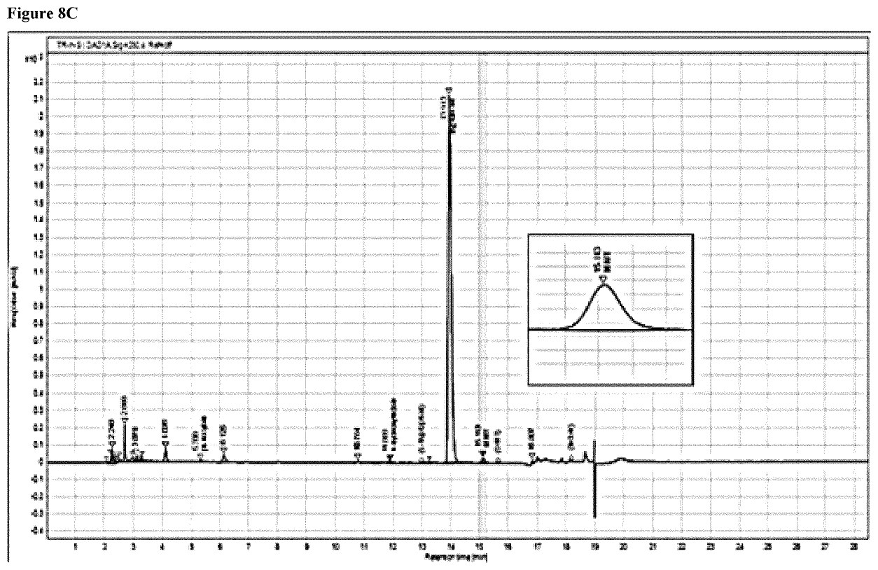

Production of DMT from L-tryptophan requires two enzymes, L-tryptophan decarboxylase, and indolethylamine-N-methyltransferase (INMT). Both were added to the E. coli genome to enable production. INMT enzymes were engineered to remove the allosteric inhibition sites to prevent feedback inhibition of synthesis. The rINMT from rabbit lung (SEQ ID NO: 8; Oryctolagus cuniculus) and the same gene with 6×his tag removed rINMT-h (SEQ ID NO: 9), the sINMT from spider (SEQ ID NO: 10; Araneus ventricosus), the wINMT from roundworm (SEQ ID NO: 11; Anyclostoma ceylanicum), and the fINMT from American bullfrog (SEQ ID NO: 12; Lithobates catesbeianus), all expressed in E. coli (FIG. 8) and tested for methylation of tryptamine in E. coli. As can be observed from FIGS. 8A and 8B, each of these constructs was expressed in E. coli. Spider sINMT (FIG. 8, lane 4) was expressed especially well. Further, FIG. 8C shows methylation by rINMT of tryptamine to produce monomethyl tryptamine (MMT), a lesser known psychedelic drug. MMT becomes active in combination with a monoamine oxidase A inhibitor.

### Selection and Optimization of Non-Pathway Genes

In addition to optimizations to the pathway related genes and enzymes discussed above, several genetic modifications have been made to the underlying producer strain or additional non-pathway related genes introduced in vectors to assist in production.

Tryptophanase (trpA) is involved in the first step of the sub-pathway that synthesizes indole and pyruvate from L-tryptophan. It is part of the degradation pathway for L-tryptophan via the pyruvate pathway, which is itself part of general amino acid catabolism. Indole acts as an autoinducer of transcription of tnaAB, astD, and gabT in E. coli. Therefore, indole signaling enhances the production of indole itself. Activities of AstD and GabT involve the production of pyruvate and succinate from the amino acid. Indole signaling also influences the multidrug exporters and biofilm formation in E. coli.

Decarboxylation of L-tryptophan and 4-hydroxytryptophan is the first step for production of either tryptamine or 4-hydroxytryptamine, respectively (FIG. 1). PsiD has been expressed in an active form in E. coli and is known to mediate the conversion of 4-OH-L-tryptophan to 4-OH-tryptamine. Thus, the normal L-tryptophan to tryptamine conversion works with no other cofactors required. However, a major barrier to production is the conversion of L-tryptophan to indole since this (i) diverts added L-tryptophan so that it cannot be utilized for psilocybin synthesis and (ii) acts as an inhibitor of the synthesis of psilocybin.

For the purpose of synthesizing both psilocybin and DMT, depletion of L-tryptophan through the production of indole is undesirable. To overcome these issues, the following host genetic background was constructed to enable optimization of PsiD activity by: (i) deletion of the critical parts of the tnaA (tryptophanase) gene from the tnaAB operon to create a ΔtnaA genotype to reduce or prevent the conversion of L-tryptophan to indole, and (ii) retention of tnaB (low affinity high capacity L-tryptophan membrane transporter) to enable the transport of L-tryptophan into cells from the cultivation media. As an alternative, a mutation leading to a frame shift in tnaA as well as inserting of three stop codons to block translation in all three reading frames (tnaAfs, a frameshift mutation) was also created. Active tnaB gene involved in L-tryptophan (and likely also 4-hydroxytryptophan) transport across the cytoplasmic membrane was maintained, since this is used catabolically and required for psilocybin biosynthesis. The essential parts of the tnaA gene (preserving all regulatory sequences) were deleted using either an efficient in vivo genome editing system or overlap extension mutagenesis.

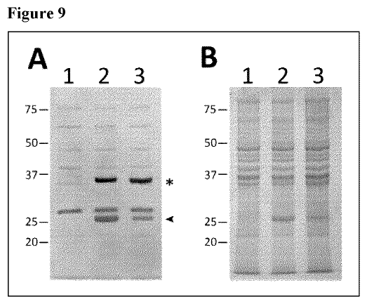

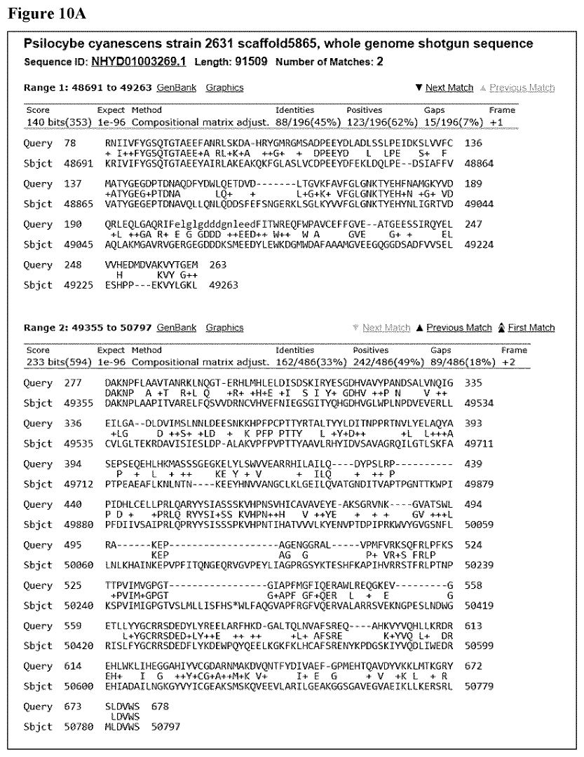

The methylation reactions catalyzed by PsiM and IMNT require the cofactor S-Adenosylmethionine (SAM), which is converted to S-adenosyl-homocysteine (SAH) known to inhibit SAM-dependent methylases. The native E. coli enzyme S-adenosyl-homocysteine nucleosidase (MTNN, gene mtnN) degrades SAH by catalyzing the cleavage of the glycosidic bond in SAH to adenine and S-ribosyl-homocysteine. To prevent or reduce inhibition of PsiM by SAH, mtnN was over-expressed. Helper plasmids were constructed carrying optimized synthetic mtnN gene (SEQ ID NO: 13) based on p15A and pSC101 plasmid replicons under L-arabinose induction control. These plasmids produced different amounts of MTNN. E. co/i TOP10 and BL21-ΔtnaA were transformed by electroporation with two plasmids capable of expressing psiM and mtnN respectively using L-arabinose induction control. Both proteins were simultaneously expressed, as verified by Western Blot (FIG. 9). This was done for all cloned INMTs.

To avoid depletion of the NADPH pool by transferring electrons to NAD⁺ growth media are supplemented with gluconate and/or cinnamyl alcohol. To ensure sufficient iron, the hemin receptor ChuA was cloned in. Hemin is important for full folding of the holoenzyme PsiH.

Catalytically active PsiH holoprotein is a heme (iron containing) protein. Even with good expression of apoenzyme (lacking heme), no enzymatic activity would be observed. The synthesis of heme is limited in E. coli. It can be induced by feeding the relatively expensive b-aminolevulinic acid (500 μmoL/L), limiting intermediate (negative feedback regulation by heme) of the heme biosynthesis pathway. Typical laboratory strains of E. coli have limited capacity to take up heme supplemented into a growth medium. However, the pathogenic strain of E. coli O157:H7 encodes a TonB-dependent outer membrane-bound heme receptor (ChuA) and is capable of heme influx. To overcome this deficiency, a chuA gene cassette (SEQ ID NO: 15) was designed and synthesized to test if it could support expression of functional holoenzyme. To maintain a working level of NADPH, growth media was supplemented with gluconate and/or cinnamyl alcohol, known substrates for the NADPH regenerating enzymes 6-P-gluconate dehydrogenase and cinnamyl alcohol dehydrogenase in E. coli. E. coli TOP10 harboring pBAD24E-chuA was cultivated in TFB with shaking at 37° C. Production of ChuA was induced by addition of 0.15% L-arabinose. If needed, downregulation of expression of ChuA may be achieved by moving the expression cassette into a low copy plasmid or decreasing the activity of the ribosomal binding site. For the former, pSC-Km was developed, based on the pSC101 replicon.

Tryptamine can be hydroxylated by cytochrome P450 tryptamine-4-monooxgenase (PsiH; SEQ ID NO: 14) from Psilocibes which introduces an oxygen atom, derived from molecular oxygen into tryptamine position 4 of the benzene ring (FIG. 1). This mono-oxygenation reaction consumes reduced nicotinamide adenine dinucleotide phosphate (NADPH), generates water. A key step in making this enzyme work is to provide NADPH-cytochrome P450 reductase (CPR) and to ensure it has sufficient NADPH to operate. Since iron is limiting to CPR enzyme activity, the hemin receptor ChuA from E. coli was added to increase iron availability.

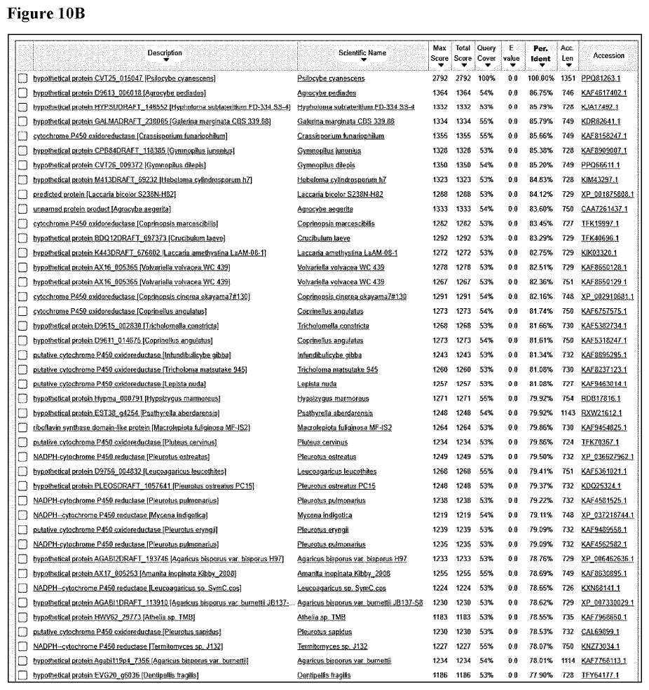

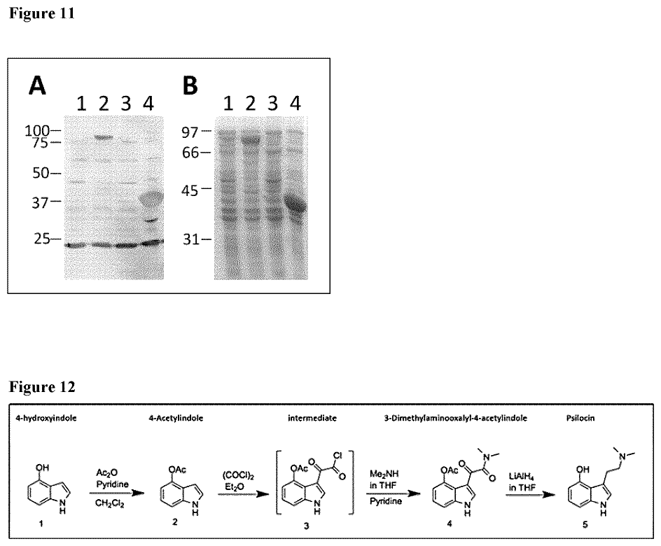

The corresponding NADPH-cytochrome P450 reductase (CPR) from the psilocybin-producing mushroom P. cyanescens is unknown. Therefore, using rCPR (SEQ ID NO: 16; Rattus norvegicus) as a starting point, a search for a suitable version of this gene was conducted. Homologs of rCPR using BLAST with the P. cyanescens genome were searched and two matches with 45% and 33% identities were found to the translated protein sequence (FIG. 10A). Both were from the same protein, a hypothetical protein CVT25_015047 which based on this homology is a putative pCPR (SEQ ID NO: 17). The first 196 amino acids showing 45% identity corresponds to the flavodoxin region of rCPR, which is the redox part of the CPR. The next 486 amino acids showed 33% identity to the NADPH cytochrome P450 reductase portion (CYPOR) of rCPR. Expression analysis (FIG. 11, lanes 1 and 3) demonstrated that neither rCPR nor pCPR were expressed in E. coli.

Subsequently, the GenBank Protein Database was searched for significant homologs of this hypothetical protein pCPR (CVT25_015047) from P. cyanescens (FIG. 10B). There were many hits among mushroom species with >50 percent identity. CPR from the common fieldcap mushroom Agrocybe pediades was selected and tested as a PsiH_PCY redox partner. This gene cassette was designated aCPR (SEQ ID NO: 18). This protein was well expressed in E. coli (FIG. 11, lane 2).

To provide an optimal background for PsiH the soluble transhydrogenase gene (sthA) is disrupted to avoid depletion of the NADPH pool by transferring electrons to NAD⁺.

E. coli are natural producers of L-tryptophan and levels can be further boosted. A moderate boost in L-tryptophan production by relaxing trp operon control is sufficient to support biosynthesis of psilocybin.

Semi-Synthetic Production from Psilocin

In addition to recombinant methods of production, efficient semi-synthetic production of psilocybin from precursor psilocin using cloned fungal kinases is also demonstrated.

For chemical synthesis of psilocin, the method used was according to FIG. 12 from Shirota et al. (Shirota, O., W. Hakamata, and Y. Goda. 2003. Concise Large-Scale Synthesis of Psilocin and Psilocybin, Principal Hallucinogenic Constituents of “Magic Mushroom. J. Natural Products 2003 66(6):885-887). Briefly, the method involves the following:
1. 4-hydroxyindole ((1); >25 g/bottle, >185 mmol) is dissolved in anhydrous CH₂Cl₂ (200 mL). In an ice bath, pyridine (20 mL, 246 mmol) and acetic anhydride (20 mL, 210 mmol) are added with constant stirring. After the mixture is stirred for 2 h at room temperature, H₂O is added, and the mixture is evaporated in vacuo. The resulting concentrate is dissolved in ethyl acetate and washed twice with H₂O and once with saturated NaCl. The organic phase is than dried over anhydrous Na₂SO₄ and the volume reduced by evaporation to form a crystalline material, which is collected by filtration and successively washed with H₂O and ethyl acetate resulting in 4-acetyoxyindole (2) (34 g; constant; ivory white crystals). This material is directly used in the next step.
2. 4-acetoxyindole (17.6 g, 100 mmol) is dissolved in anhydrous diethyl ether (100 mL) with stirring in an ice bath and oxalyl chloride (13 mL, 146 mmol) is added. After stirring for 15 min, n-hexane (200 mL) is added, and the reaction flask is placed in a freezer overnight. The resulting yellow crystals (3) are separated from the solution by filtration and dissolved in anhydrous tetrahydrofuran (100 mL). 2M dimethylamine tetrahydrofuran solution (60 mL, 120 mmol) and pyridine (10 mL, 123 mmol) are added over 15 min with stirring in an ice bath. Additional anhydrous ether is then added to the mixture because of solidification and stirring continues for 15 min at room temperature. The reaction product is then filtered out and successively washed with n-hexane, ethyl acetate, and H₂O, resulting in crystals of 3-dimethylaminooxalyl-4-acetylindole (4).
3. To a suspension of lithium aluminum hydride (ca. 12 g) in anhydrous tetrahydofuran (300 mL) under an argon atmosphere add dropwise a solution of 3-dimethylaminooxalyl-4-acetylindole (22.0 g, 80 mmol) in anhydrous tetrahydofuran (250 mL) over 2 h. Reflux the reaction mixture for 2 h. After cooling, add anhydrous Na₂SO₄ powder (ca. 10 g) and then add dropwise solution of saturated Na₂SO₄ (ca. 12 mL) over 1 h period with stirring at room temperature. After the reaction is stopped, add additional anhydrous Na₂SO₄ powder (ca. 10 g). Dilute the reaction mixture with ethyl acetate and filter through an aminopropyl silica gel laminated Celite pad by suction. Wash the pad with ethyl acetate. Concentrate the organic solution in vacuo and wash the resulting crystals briefly with MeOH to yield psilocin (5).

The skilled person will appreciate that other methods of chemical synthesis to produce psilocin may also be utilized for downstream processing with the kinases discussed below.

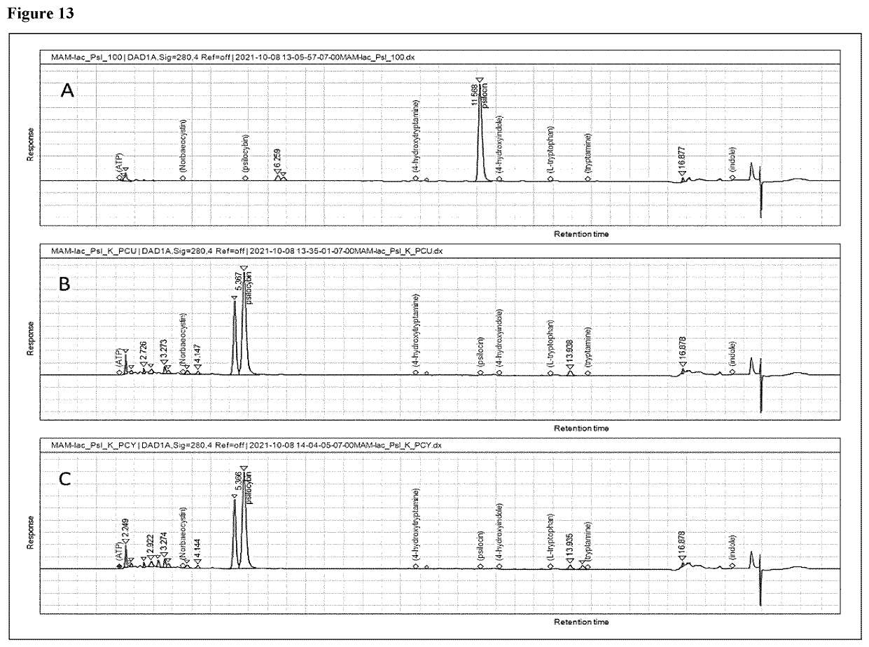

Both kinases, PsiK_PCY (SEQ ID NO: 4) and PsiK_PCU (SEQ ID NO: 5), phosphorylated psilocin to produce psilocybin (FIGS. 13A to 13C). Thus, a clear semisynthetic route to producing psilocybin is to utilize psilocin as a precursor and use one or both of these two E. coli-optimized genes to produce psilocybin with virtually no contaminants and nearly quantitatively (the peak in front of psilocybin has a peptide-like spectrum and is the result of fermentation; it is easily removable). Since psilocin is relatively inexpensively produced this represents a novel semi-synthetic method for producing psilocybin at lower cost.

### Expression Control—Vector Design

DNA was synthesized in a format of gene cassettes allowing construction of expression plasmids (as well as great flexibility in movement from one plasmid to another) and finally to create functional operons as required for designated production strategies.

pBAD24E

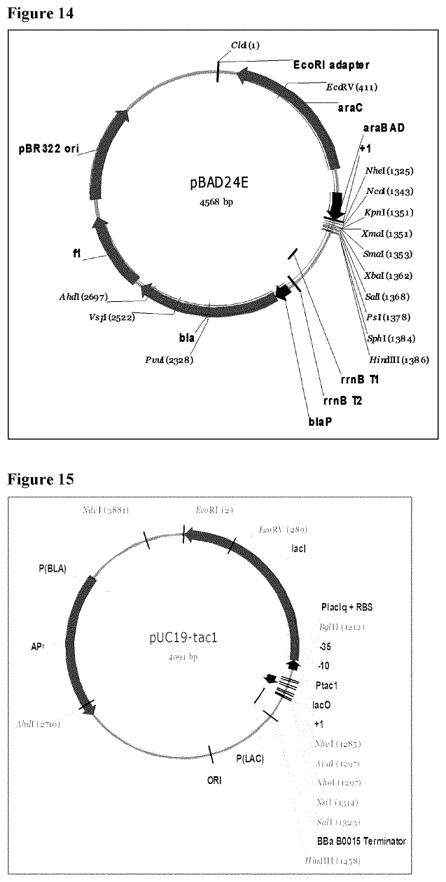

Initial gene expression testing was carried out in the pBAD24E vector (FIG. 14). The genes for each protein were designed to contain a six histidine tag for easy Western blot detection. All gene cassettes (SEQ ID NOs: 1-17) were obtained as clones in plasmid pUC57 Km (Biobasic). Each of these gene cassettes was designed with NheI followed by XhoI (5′ end) and Sail (3′ end) restriction enzymes sites (recognition sequences). A ribosome binding site (RBS) resides between the XhoI and NsiI restriction sites just upstream of the ATG start codon and was designed to be easily replaceable with other variants if chosen. The chosen configuration of restriction sites allowed for the combining of relevant gene cassettes into desired operons as follows. The first cassette was ligated into pBAD24E as an NheI-SalI DNA fragment using the same plasmid cloning sites in this vector, pBAD24E. Subsequent cassettes could then be inserted into plasmids digested with NheI & XhoI restriction enzymes as NheI-SalI fragments because ligation of Sail & XhoI sticky ends removes any recognizable restriction site. Since this cloning also always brought a new XhoI restriction site into expression plasmid, the procedure could be repeated as many times as required. Expression is fully induced by addition of 0.2% arabinose and is relatively highly repressed in the presence of glucose.

pUC19-tac

Synthetic DNA fragment encoding for lacl^{q}, promoter tac and MCS was ligated between EcoRI and HindIII restriction sites of plasmid pUC19 resulting in a high copy plasmid (FIG. 15). Despite the presence of the lacl^{q} gene in the plasmid, expression is not fully repressed. Expression can be further increased by addition of IPTG, also in the presence of glucose.

p15A-Km

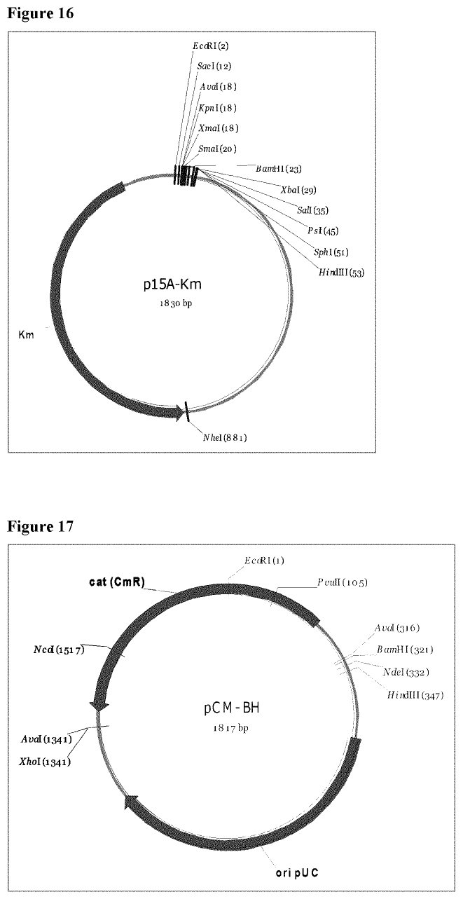

The p15A-Km vector is a synthetic plasmid with a p15A origin of replication, MCS from pUC18 and has an optimized kanamycin resistance gene (FIG. 16). This plasmid has a relatively low copy number (˜15) and was used to construct low copy analogs of pUC19-tac1 clones.

pCM-BH

This plasmid was created by ligation of two PCR products. A fragment with chloramphenicol resistance gene (cat) (823 bp) and a 1341 bp fragment with pUC type origin of replication, resulting in a high copy plasmid (˜600) (FIG. 17). This vector was used as an intermediate vector for transfer of operons from pBAD24E based constructs to pUC19-tac1.

pKLPR-N

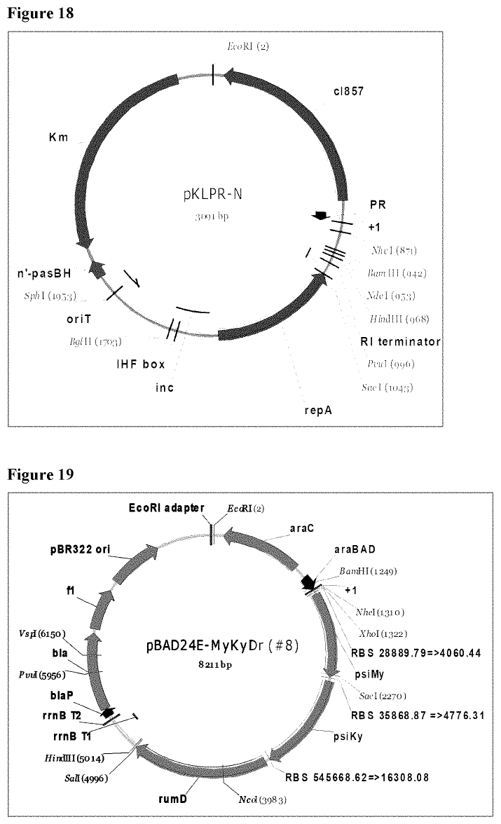

The pKLPR-N vector is a synthetic plasmid with kanamycin selection marker based on the pKL1 replicon (FIG. 18) and has a higher copy number than pBAD24E. This plasmid was designed to induce expression with temperature shift from a lambda phage promoter controlled by C1875 thermosensitive repressor.

### Arabinose Induction

L-arabinose induction was employed to control expression in various production strains. Induction through L-arabinose allows rapid repression of expression by addition of glucose to the cultivation medium at any time. When glucose is consumed and L-arabinose is still present, expression is once again induced. For production purposes, the host strain was engineered to not degrade L-arabinose and to be able to support modulation of expression from the pBAD promoter. Unless otherwise indicated, enzyme production was induced by the addition of 0.15% L-arabinose.

Operon araBAD encodes three enzymes involved in the degradation of L-arabinose: ribulokinase (AraB), L-arabinose isomerase (AraA), and L-ribulose 5-phosphate 4-epimerase (araD). AraA is involved in the first step of the sub-pathway that synthesizes D-xylulose 5-phosphate from L-arabinose. The araA gene was deleted to disrupt this pathway and stop degradation of L-arabinose.

The transcriptional regulator AraC controls both the L-arabinose catabolic genes (araBAD) and transporter genes (araE and araFGH located at different coordinates on the chromosome) via L-arabinose-based induction. The L-arabinose-inducible araBAD promoter (pBAD) induces gene expression in an on/off fashion. As such, the proportion of induced and uninduced cells in the population is altered, rather than modulation of expression levels in individual cells. Thus, the number of induced cells fluctuates with the concentration of L-arabinose in the culture medium. This type of induction of PBAD is due to the L-arabinose induced expression of the gene encoding the low-affinity, high-capacity L-arabinose transporter (araE) as well as high affinity, low-capacity transporters encoded by operon araFGH. Therefore, expression of genes under PBAD control in individual cells can be regulated by controlling araE gene with an L-arabinose-independent promoter, especially if araFGH operon is inactivated. Higher and constant AraE concentrations in cells results in a much lower L-arabinose concentration required to induce the pBAD promoter and allows modulation of expression in individual cells by increasing/decreasing L-arabinose concentration in a culture media. Therefore, several modifications were made to allow for such control in the production strain, such as creation of AaraFGH & (ΔPE ΔRBS-araE):p119-RBS_{syn}. The native araE promoter was replaced with a consensus E. coli promoter, designated p119. This promoter has a moderate constitutive activity, which can be too strong if used in high-copy vectors. A computer algorithm was used to evaluate the activity of the native ribosome binding site (RBS) of the araE gene. Based on this, to further modulate expression of araE, the original RBS-araE was replaced with two different RBS sequences having respective relative activities 9.6× and 47.3× higher than the native RBS-araE predicted activity. Both RBSs were designed specifically for araE ORF to result in a constant and higher concentration of AraE transporter, thus allowing efficient modulation of expression by increasing L-arabinose concentration.

The psiD gene was also cloned into pBAD24 under arabinose control since induction must be done in the absence of L-glucose, which also avoids catabolic repression of TnaB. This allows the resulting strain harboring pBAD24-psiD to work optimally when enzyme activity is analyzed by HPLC comparing to indole and tryptamine standards for the method calibration. The following parameters were used in HPLC analysis:
Column: Zorbax Eclipse 5 XDB-C18 250×4.6 mm
Solution A: HPLC grade water, 0.1% TFA
Solution B: HPLC grade Acetonitrile
Gradient timetable:

| Time [min] | A[%] | B[%] |
|---|---|---|
| 0 | 95 | 5 |
| 1 | 95 | 5 |
| 13 | 81 | 18 |
| 16 | 0 | 100 |
| 16.01 | 95 | 5 |
| 28.46 | 95 | 5 |

The components in the MAM media (per liter) used for cell culture growth are as follows: 800 mL Base Media (3.5 g/L KH₂PO₄, 5 g/L K₂HPO₄, 3.5 g/L (NH₄)₂HPO₄, 2 g/L casamino acids, 6.25 g/L L-methionine, 3.125 g/L L-serine), 100 mL of 10×MOPS Mix (83.7 g/L MOPS, 7.2 g/L Tricine, 28 mg/L FeSO₄×7 H2O, 29.2 g/L NaCl, 1.1 g/L MgCl₂, 0.5 g/L K₂SO₄), 1 mL 1M MgSO₄, 0.1 mL 1M CaCl₂), 1 mL 0.5 g/L Thiamine hydrochloride (optional for deficient strains), 0.2 mL Micronutrient stock (0.2 g/L (NH₄)₆Mo₇O₂₄, 1.2 g/L H₃BO₃, 0.1 g/L CuSO₄, 0.8 g/L MnCl₂, 0.1 g/L ZnSO₄), and 100 mL sugar solution (20% glucose or 15% lactose).

To enable psilocybin synthesis and allow modulation of expression of relevant operons with arabinose, all constructs were expressed in E. coli BL21-ΔtnaA-ΔaraA. Abbreviations for genes used in creation of the plasmids discussed below are specified in the following Table 1:

| Ab- | | | Gene | |
|---|---|---|---|---|
| brevi- | Gene | Original Strain | Cassette | |
| ation | Name | Source | Sequence | ORF Sequence |
| Dr | rumD | Ruminococcus gnavus | SEQ ID NO: 2 | SEQ ID NO: 21 |
| My | psiM | Psilocibe cyanescens | SEQ ID NO: 6 | SEQ ID NO: 25 |
| Mu | psiM | Psilocibe cubensis | SEQ ID NO: 7 | SEQ ID NO: 26 |
| Ky | psiK | Psilocibe cyanescens | SEQ ID NO: 4 | SEQ ID NO: 23 |
| Ku | psiK | Psilocibe cubensis | SEQ ID NO: 5 | SEQ ID NO: 24 |

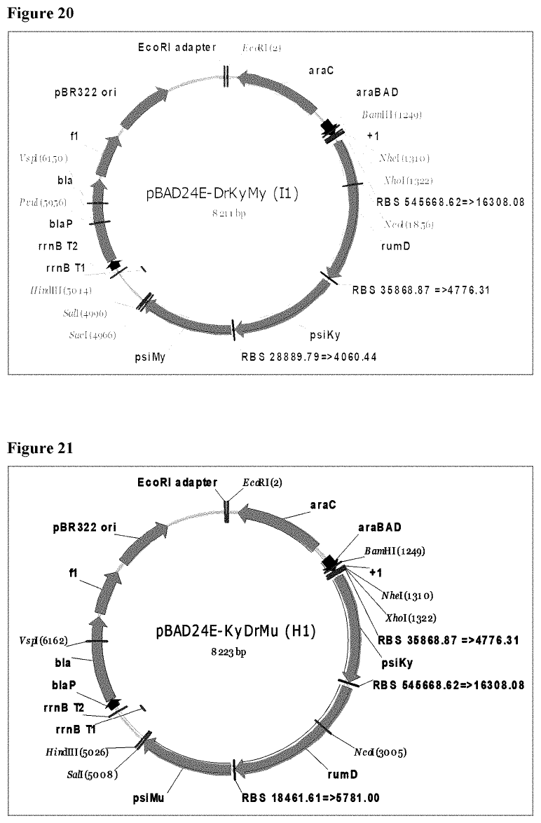

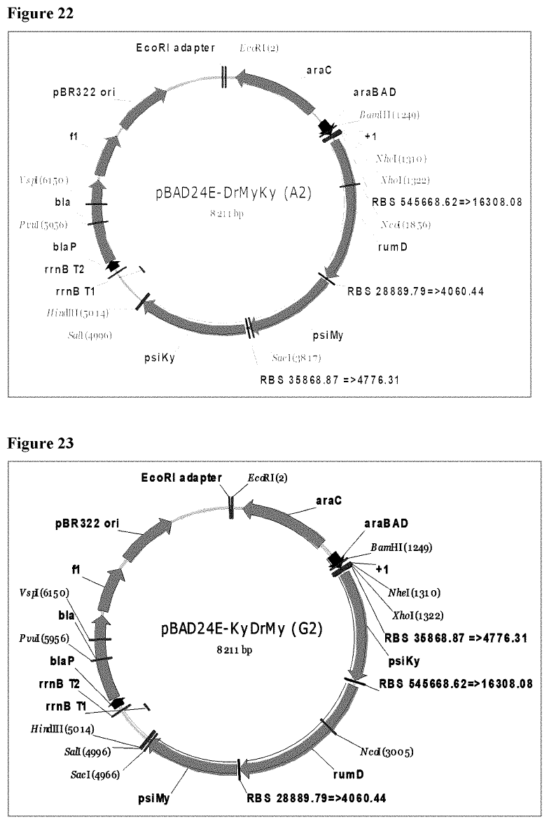

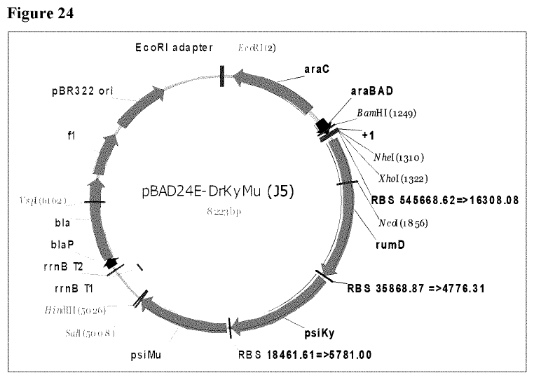

Six plasmids based on pBAD24E containing combinations of three relevant genes in different order and alternated genes PsiM-My and -Mu were constructed: pBAD24E-MyKyDr (FIG. 19), pBAD24E-DrKyMy (FIG. 20), pBAD24E-KyDrMu (FIG. 21), pBAD24E-DrMyKy (FIG. 22), pBAD24E-KyDrMy (FIG. 23) and pBAD24E-DrKyMu (FIG. 24). Each individual gene was designed and optimized to allow expression of the cloned gene in E. coli, and expression levels were verified by Western blots developed with anti-his tag polyclonal serum. 4-hydroxytryptamin kinase PsiK (Ky) and tryptophan decarboxylase RumD (Dr) were selected because they exhibited increased activity. However, other kinases (e.g., Ku) and decarboxylases (e.g., PsiD and CloD) may also be used in plasmids for psilocybin production as well.

Six starter cultures with these six “pBAD24E-operon” plasmids were grown to the late exponential phase in Terrific Broth containing 0.2% glucose and 300 μg/mL of ampicillin. Starter cultures were used to inoculate (2% inoculum) MAM-2%-glucose media (repressed expression from pBAD24E) containing 300 μg/mL of ampicillin overnight. These cultures were used to inoculate (10% inoculum) MAM-1.5%-lactose media containing 300 μg/mL of ampicillin and 150-200 μg/mL of 4-hydroxyindole. All cultures with 4-hydroxyindole were cultivated for 20 hours. All cultivation from start to finish was carried out in tubes filled with 1.5 mL media, 37° C. and shaking at 250 rpm. Induction of expression was achieved by addition of 0.015% or 0.15% arabinose. Final concentration of psilocybin in MAM-lactose media was determined by HPLC analysis and these results are summarized in Table 2 below.

TABLE 2
Concentration of psilocybin produced by BL21-ΔtnaA-
ΔaraA harbouring single plasmids: pBAD24E-MyKyDr,
pBAD24E-DrKyMy, pBAD24E-KyDrMu, pBAD24E-DrMyKy,
pBAD24E-KyDrMy or pBAD24E-DrKyMu.

| | |
|---|---|
| 0.015% arabinose | 0.15% arabinose |

| | |
|---|---|
| Operon | Concentration of Psilocybin (μg/mL) |

| | | |
|---|---|---|
| MyKyDr | ND | ND |
| DrKyMy | 12.0 | 17.0 |
| KyDrMu | 23.5 | 15.8 |
| DrMyKy | 59.7 | 34.2 |
| KyDrMy | 73.2 | 34.3 |
| DrKyMu | 60.6 | 96.7 |

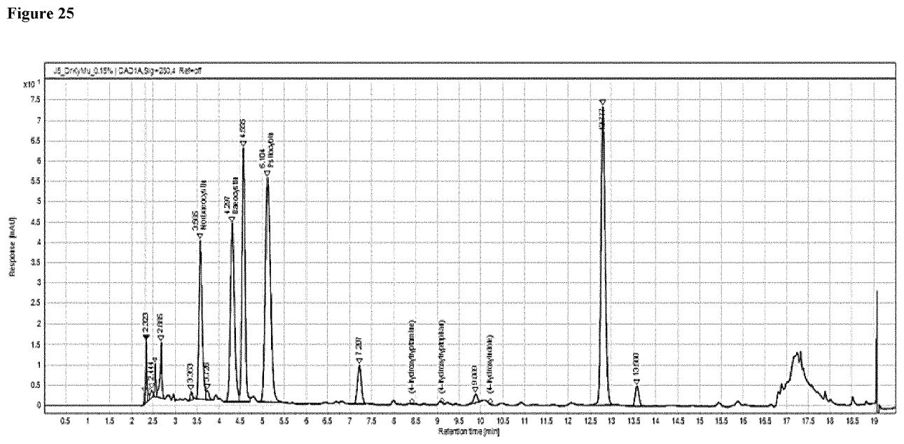

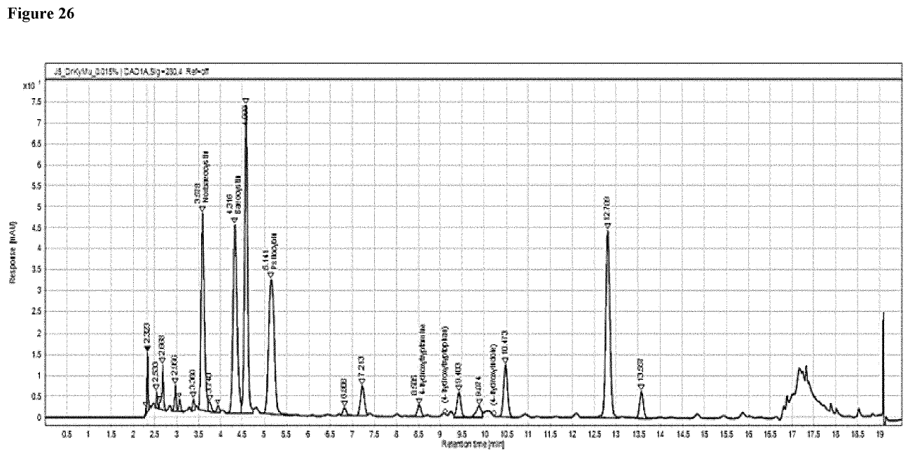

The operon “DrKyMu” produced significantly higher amounts of psilocybin and other intermediates: 96.7 μg/mL of psilocybin, 36.6 μg/mL of Norbaeocystin and 49.9 μg/mL of Baeocystin at 0.15% arabinose (FIG. 25) and 60.6 μg/mL of psilocybin at 0.015% arabinose (FIG. 26). A significant amount of Tryptamine was also detected with operon DrKyMu. The gene product of psiD (Dr) in the operon also decarboxylated L-tryptophan produced by host cells to tryptamine after depletion of 4-hydroxytryptophan. No 4-hydroxytryptophan and 4-hydroxytryptamine were detected when expression was induced with 0.15% arabinose suggesting that constant feeding with 4-hydroxyindole is possible without accumulating 4-hydroxytryptophan and 4-hydroxytryptamine.

The plasmids with operons “KyDrMu”, “DrMyKy”, and “KyDrMy” achieved higher concentrations of psilocybin with 0.015% arabinose than 0.15% arabinose as inducer. These results suggest that induction of operon expression that is too high in some cases may be detrimental for the conversion process. High expression can lead to misfolding and denaturation of produced proteins in E. coli.

### Plasmid Copy Number

Operon expression levels may be modulated via plasmid copy number. Expression vectors for operons “DrKyMu” and “KyDrMy” with copy number higher and lower than pBAD24E were constructed based on the different origins of replication of plasmids pKLPR-N(high copy number) and p15A-Km (low copy number).

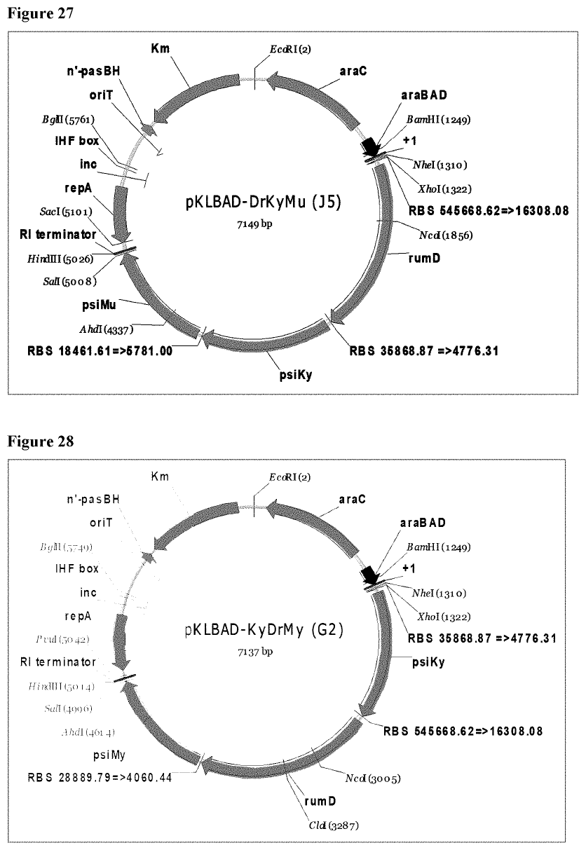

Plasmid pKLPR-N was cut with EcoRI and HindIII and ligated with EcoRI-HindIII fragments of pBAD24E-DrKyMu and pBAD24E-KyDrMy resulting in plasmids pKLBAD-DrKyMu (FIG. 27) and pKLBAD-KyDrMy (FIG. 28), respectively.

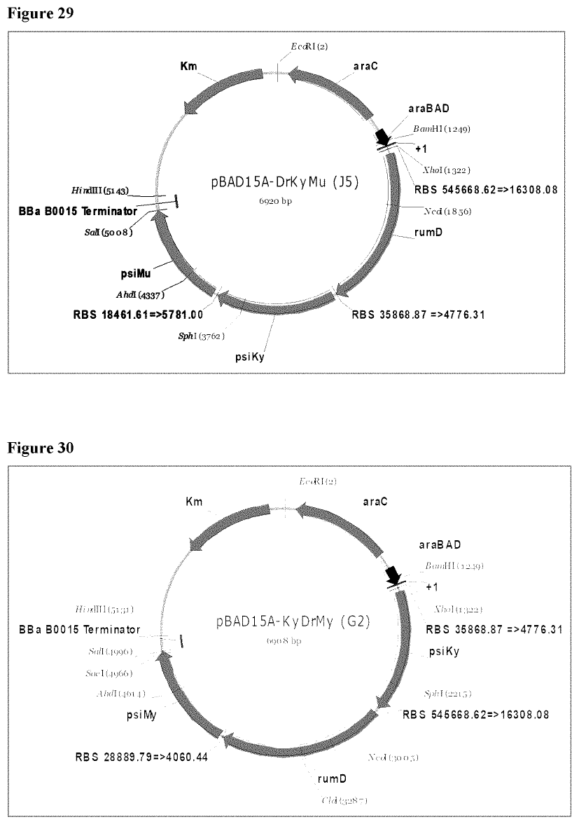

Plasmids based on p15A-Km replicon were constructed from p15Al^{q}tac1-DrKyMu and p15Al^{q}tac1-KyDrMy by cutting them with EcoRI and XhoI and inserting EcoRI-XhoI fragment from pBAD24E-DrKyMu and pBAD24E-KyDrMy containing arabinose induction system resulting in pBAD15A-DrKyMu (FIG. 29) and pBAD15A-KyDrMy (FIG. 30), respectively.

BL21-ΔtnaA-ΔaraA was transformed individually with all four described plasmids. Starter cultures were grown to the late exponential phase in Terrific Broth containing 0.2% glucose and 50 μg/mL of kanamycin. Starter cultures were used to inoculate (2% inoculum) MAM-2%-glucose media (repressed expression from pBAD24E) containing 50 μg/mL of kanamycin overnight. These cultures were used to inoculate (10% inoculum) MAM-1.5%-lactose media containing 50 μg/mL of kanamycin and 150-200 μg/mL of 4-hydroxyindole. All cultures with 4-hydroxyindole were cultivated for 20 hours. All cultivation from start to finish was carried out in tubes filled with 1.5 mL media, 37° C. and shaking at 250 rpm. Induction of expression was achieved by addition of 0.0015%, 0.015%, or 0.15% arabinose. The final concentration of psilocybin in MAM-lactose media was determined by HPLC analysis.

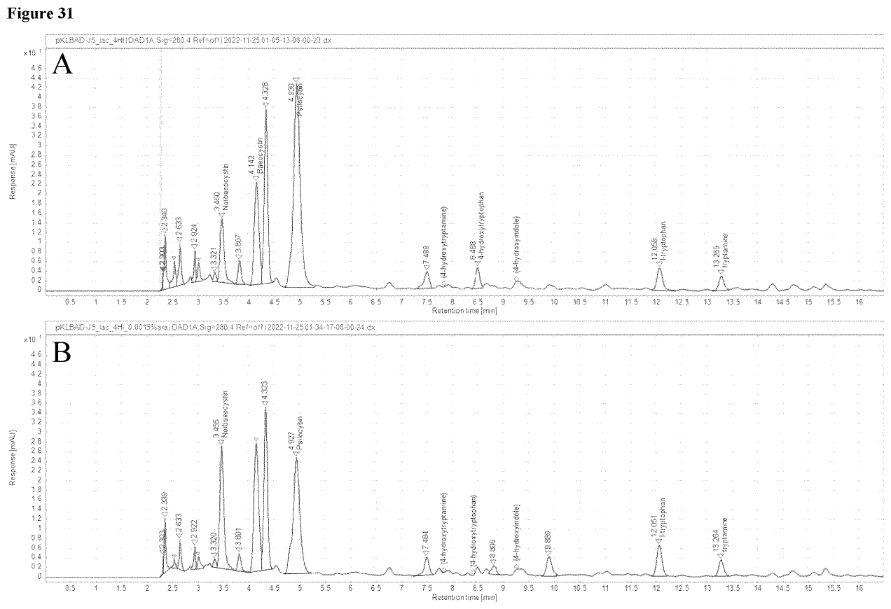

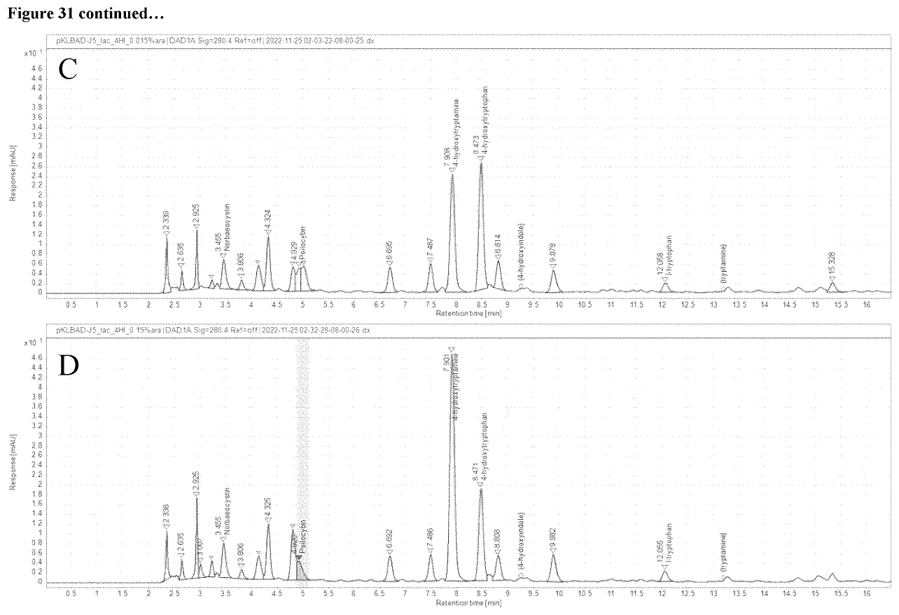

The results from BL21-ΔtnaA-ΔaraA(pKLBAD-DrKyMu) are shown in FIGS. 31A-D. This strain produced 82.9 μg/mL of psilocybin, 14.6 μg/mL of Norbaeocystin and 25.0 μg/mL of Baeocystin in the absence of arabinose. MAM-1.5%-lactose media (no arabinose) did not repress expression and any addition of arabinose significantly decreased production of psilocybin. Therefore, this strain can be used as is in MAM-lactose media based on the constitutive expression level. Strain BL21-ΔtnaA-ΔaraA(pKLBAD-KyDrMy) produced a maximum 22.1 μg/mL of psilocybin with addition of 0.0015% arabinose, strain BL21-ΔtnaA-ΔaraA(pBAD15A-DrKyMu) produced 38.8 μg/mL of psilocybin in the presence of 0.015% arabinose, and BL21-ΔtnaA-ΔaraA(pBAD15A-KyDrMy) produced 11.1 μg/mL of psilocybin in the presence of 0.15% arabinose. None of these strains produced psilocybin in the absence of arabinose.

### Induction in the Presence of Glucose

Alternative expression systems that allow the use of glucose during induction were constructed to provide for higher density cell growth and psilocybin production. These systems are based on tac1 promoter repressed by a repressor encoded by lacl^{q} and PR promoter from lambda phage repressed by a repressor encoded by cI857. These systems can by induced by the addition of IPTG or temperature shift, respectively. The strain BL21-ΔtnaA-ΔaraA was transformed individually with the plasmids described below.

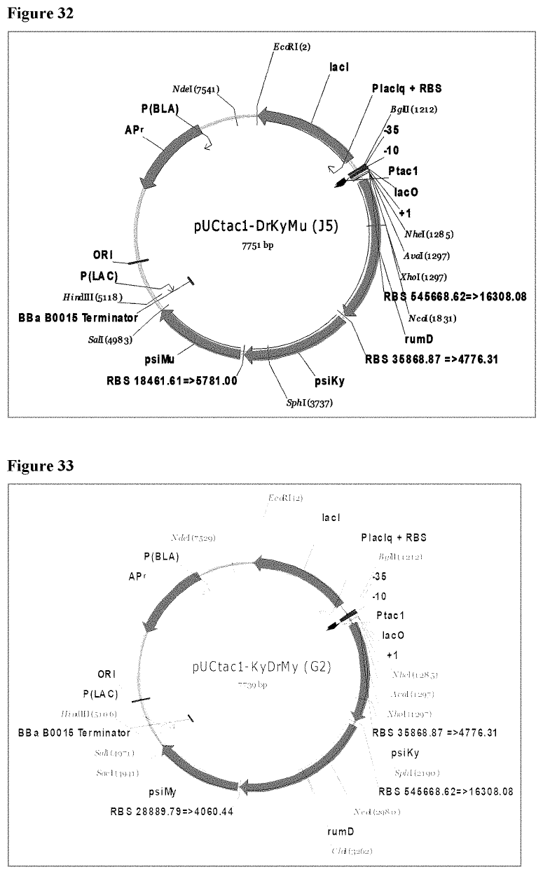

Plasmid pUC19tac1 is a high copy plasmid. Even though the lacI^{q} gene is present in the plasmid, expression from tac1 promoter is not repressed. Expression can be further manipulated by addition of IPTG, also in the presence of glucose. Operons “DrKyMu” and “KyDrMy” were first re-cloned into pCM-BH as BamHI-HindIII fragments of pBAD24E-DrKyMu and pBAD24E-KyDrMy, respectively. Both pUC19-tac1 and pBAD24E have ampicillin selection markers and pCM-BH has a chloramphenicol selection marker that allows easy cloning to pUC19-tac1 without the need to purify DNA restriction fragments. Resulting plasmids pCM-DrKyMu and pCM-KyDrMy were donors of NheI-SalI fragments that were ligated between pUC19-tac1 NheI and SalI sites resulting in plasmids pUCtac1-DrKyMu (FIG. 32) and pUCtac1-KyDrMy (FIG. 33).

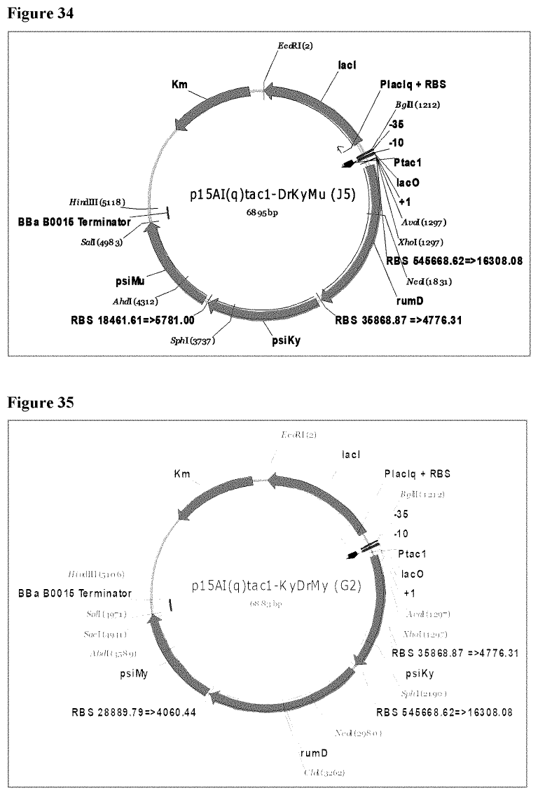

Low copy number equivalents of these plasmids were prepared in p15A-Km. Plasmid p15A-Km was cut with EcoRI and HindIII and ligated with EcoRI-HindIII fragments of pUCtac1-DrKyMu and pUCtac1-KyDrMy containing lacl^{q}-tac1-operon resulting in p15Al^{q}tac1-DrKyMu (FIG. 34) and p15Al^{q}tac1-KyDrMy (FIG. 35), respectively. The low copy number nature of these plasmids allows for better modulation of expression with IPTG in the presence of glucose.

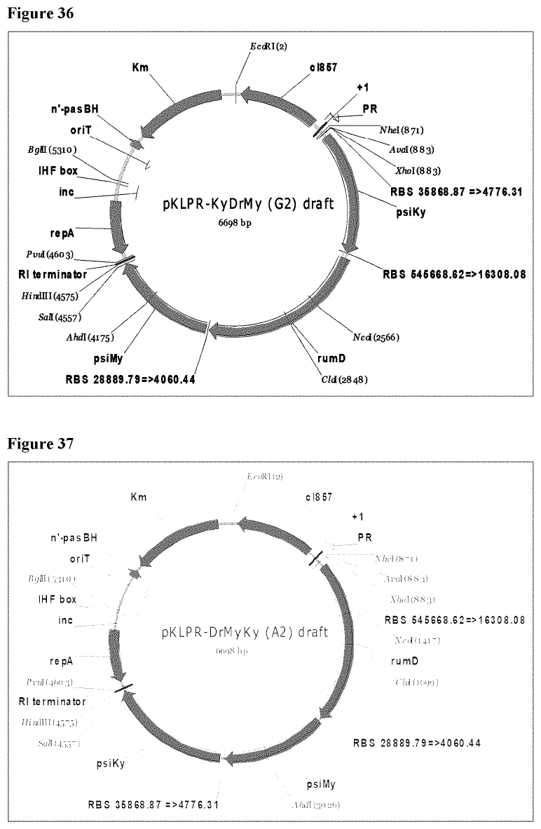

Plasmid pKLPR-N was cut with NheI and HindIII and ligated with NheI-HindIII fragments of pBAD24E-DrKyMu, pBAD24E-KyDrMy and pBAD24E-DrMyKy resulting in pKLPR-DrKyMu, pKLPR-KyDrMy (FIG. 36) and pKLPR-DrMyKy (FIG. 37), respectively.

For growth and cultivation of pUCtac1-based plasmids starter cultures were grown to the late exponential phase in Terrific Broth containing 0.2% glucose and 300 μg/mL of ampicillin or 50 μg/mL of kanamycin, depending on the plasmid. Starter cultures were used to inoculate (2% inoculum) MAM-2%-glucose media that were cultivated overnight. These cultures were used to inoculate (10% inoculum) MAM-2%-glucose media containing 150-200 μg/mL of 4-hydroxyindole. All cultivation from start to finish was carried out in tubes filled with 1.5 mL media, at 37° C. and shaking at 250 rpm. Expression was modulated by addition of IPTG—0.001 mM, 0.01 mM, or 1 mM.

For growth and cultivation of pKLPR-N based plasmids, starter cultures were grown at 28° C. to the late exponential phase in Terrific Broth containing 0.2% glucose and 50 μg/mL of kanamycin. Starter cultures were used to inoculate (2% inoculum) MAM-2%-glucose media containing 50 μg/mL of kanamycin and cultivated overnight at 28° C. These cultures were used to inoculate (10% inoculum) MAM-2%-glucose media containing 50 μg/mL of kanamycin and 150-200 μg/mL of 4-hydroxyindole.

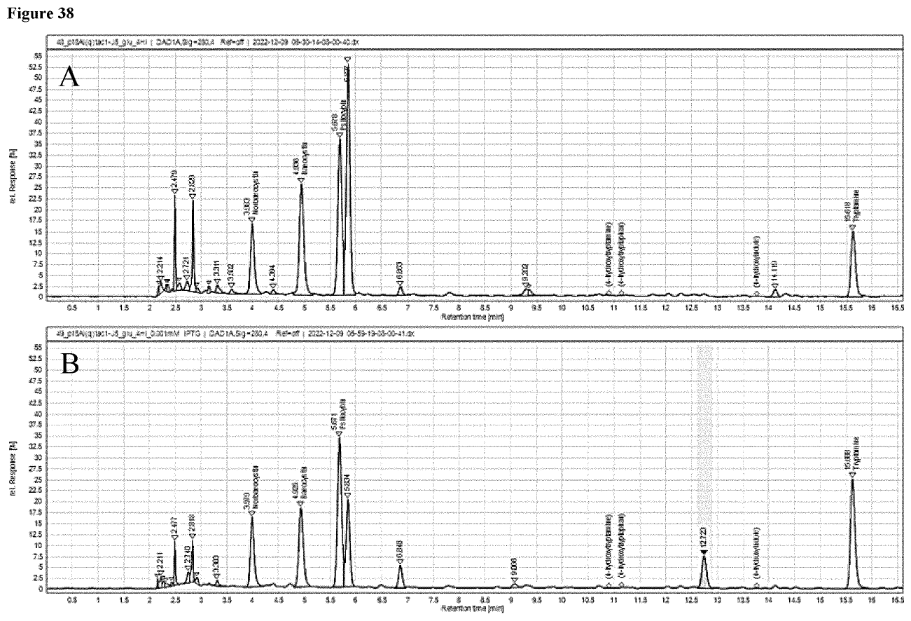

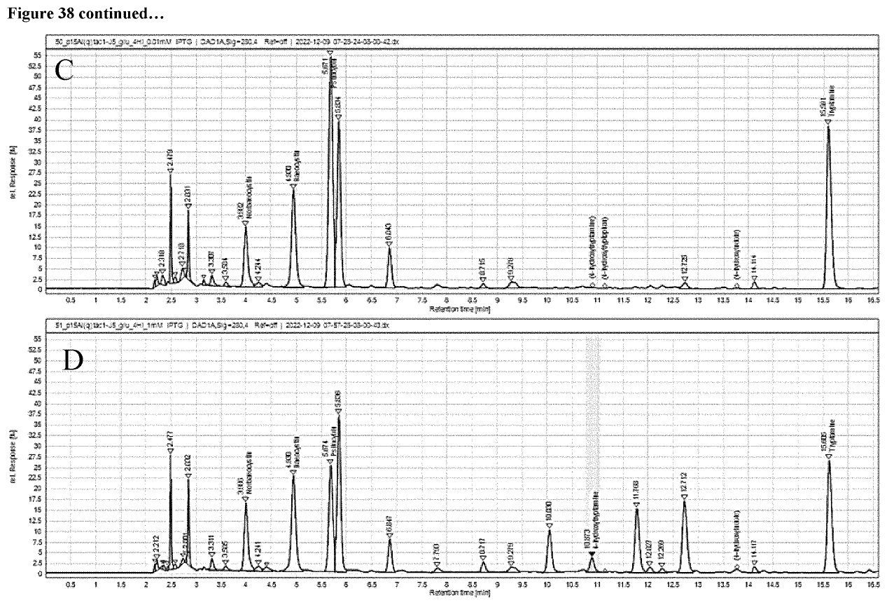

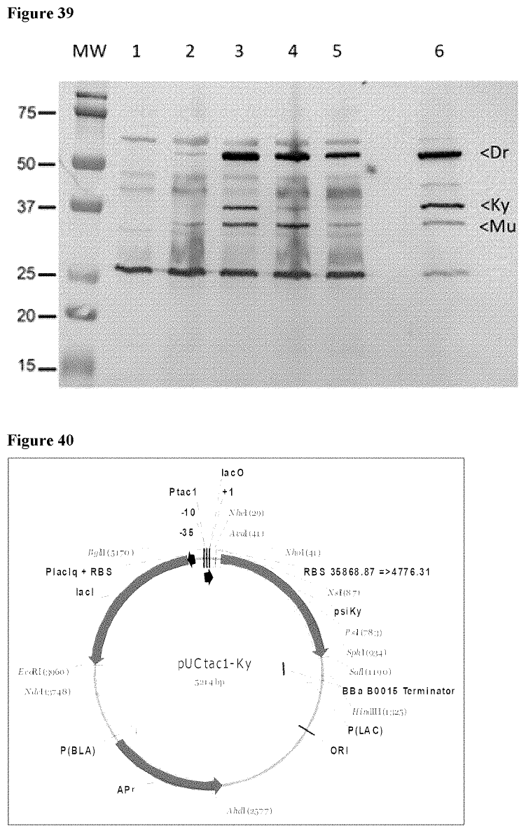

The final concentration of psilocybin in MAM-glucose media was determined by HPLC analysis. E. coli BL21 ΔtnaA ΔaraA (p15Al^{q}tac1-DrKyMu) achieved conversion of 4-hydroxyindole to psilocybin of all tested strains with significant growth in MAM-2%-glucose media (FIGS. 38A-D). More particularly, addition of 0.01 mM IPTG resulted in the production of 103.9 μg/ml of psilocybin. Without IPTG, 65.4 μg/ml of psilocybin was produced. The Western blot showing gene expression at various concentrations of IPTG of BL21-ΔtnaA-ΔaraA(p15Al^{q}tac1-DrKyMu) is shown in FIG. 39. The presence of tryptamine indicated that in all tested conditions, 4-hydroxytryptophan decarboxylase (Dr) was underutilized and more 4-hydroxyindole was required.

BL21-ΔtnaA-ΔaraA (p15Al^{q}tac1-KyDrMy) in the absence of IPTG produced 39 μg/ml of psilocybin. Addition of IPTG did not boost production, rather lowered it slightly.

### Conversion of Psilocin to Psilocybin

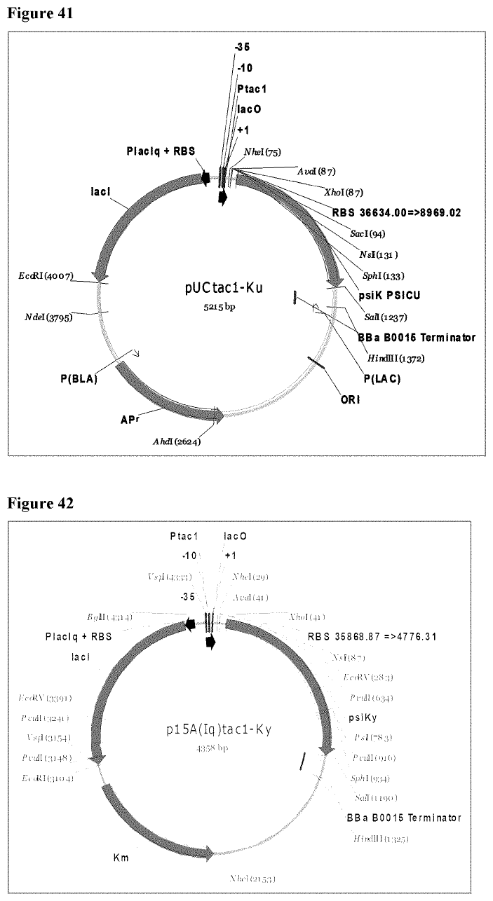

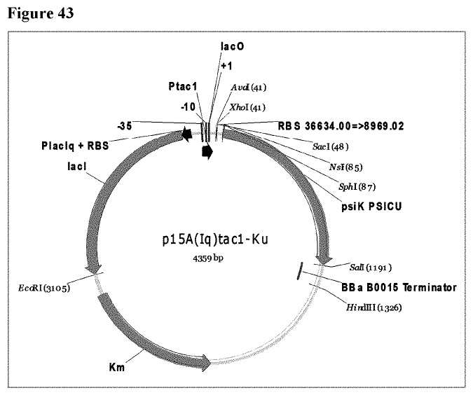

As discussed above, plasmids pBAD24E-Ky and pBAD24E-Ku converted psilocin to psilocybin (FIGS. 13A-C). Additional expression plasmids based on pUCtac1 expressing kinases Ky and Ku were constructed by ligation of NheI and SalI cut pUCtac1 and NheI-SalI fragment of PsiK_PCY gene cassette (Ky) or PsiK_PCU gene cassette (Ku) resulting in pUCtac1-Ky (FIG. 40) and pUCtac1-Ku (FIG. 41), respectively. Low copy analogues were constructed by ligation of p15A-Km cut with EcoRI and HindIII and the EcoRI-HindIII fragment of pUCtac1-Ky or pUCtac1-Ku resulting in p15Al^{q}tac1-Ky (FIG. 42) and p15Al^{q}tac1-Ku (FIG. 43), respectively.

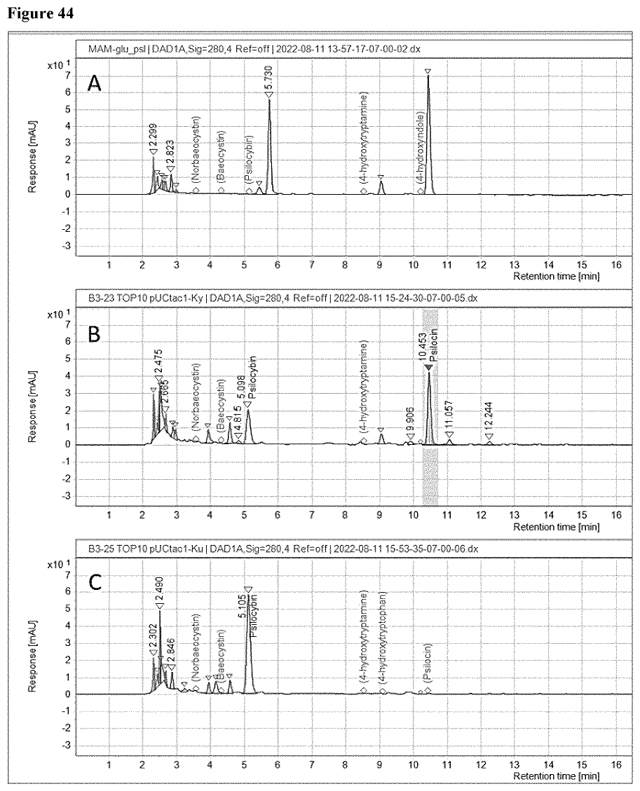

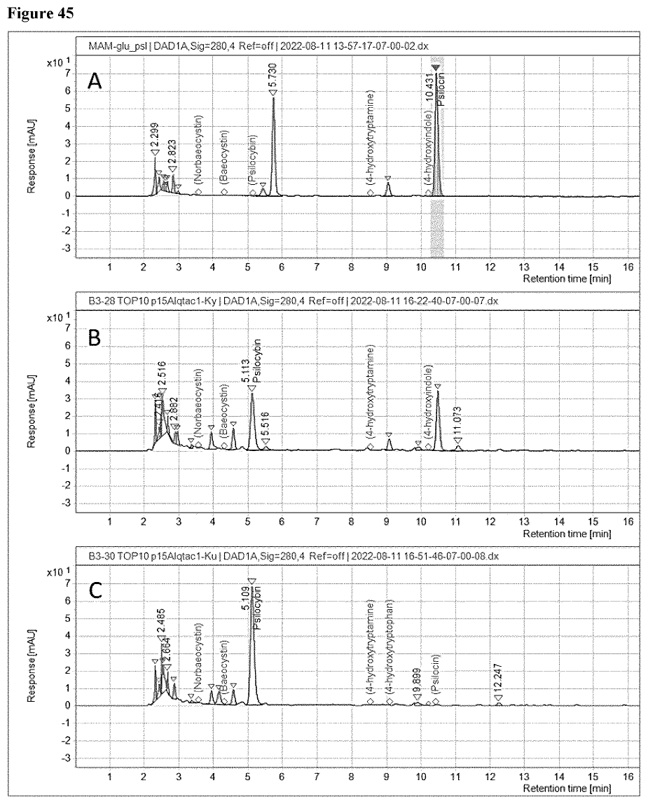

All of these plasmids allow use of glucose and expression can be modulated by addition of IPTG. Measured amounts of psilocybin for strains BL21(pUCtac1-Ky) and BL21(p15Al^{q}tac1-Ky) were 34.1 μg/mL and 53.0 μg/mL, respectively. Strains BL21(pUCtac1-Ku) and BL21(p15Al^{q}tac1-Ku) showed basal expression level that was enough to convert 100 μg/mL of psilocin to psilocybin in 19 hours and measured amounts of psilocybin were 96.5 μg/mL and 103 μg/mL, respectively. These results show that both kinases, Ky and Ku, convert psilocin to psilocybin under conditions that allow the use of glucose. HPLC results are shown in FIGS. 44A-C and 45A-C).

### Culture Media

Overproduction of L-tryptophan can also be supported by the addition of anthranilate into the medium. Anthranilic acid is produced industrially, and is also an inexpensive intermediate in the production of azo dyes and saccharin.

Terrific Broth is supplemented with additives such as bacto-peptone (2 g/L), thiamine (to 1 mmol/L), trace elements (FeCl₃, ZnCl₂, CoCl₂, Na₂MoO₄, CaCl₂), CuCl₂, H₃BO₃ and the appropriate antibiotic for selection of bacteria containing the expression plasmid. Haem supplements such as b-aminolevulinic acid (500 μmoL/L) are helpful in many cases. 2×YT and LB media may also be used. To maintain a working level of NADPH, media is supplemented with gluconate and/or cinnamyl alcohol, substrates for NADPH regenerating enzymes 6-P-gluconate dehydrogenase and cinnamyl alcohol dehydrogenase in E. coli. A temperature below 30° C. and as low as 20° C. is used for better P450 holoprotein expression. At 37° C., apoprotein expression might be elevated but little haemoprotein can be detected and recombinant P450 might be mostly localized in inclusion bodies, likely as aggregates of denatured protein. Without wishing to be bound by theory, cooling is believed to slow the expression of the protein sufficiently to allow time for proper folding and haem incorporation. Aeration of cultures is also provided which affects both bacterial growth and recombinant protein expression, and the skilled person is familiar with techniques for aeration.

Accordingly, the skilled person understands that aspects of the disclosure pertain to a recombinant microbial cell comprising a biosynthetic pathway for producing psilocybin, or intermediates thereof. The microbial cell comprises a heterologous nucleic acid encoding one or more psilocybin production genes. The one or more psilocybin production genes is a tryptophan decarboxylase, a phosphotransferase, a methyltransferase, a monooxygenase, or a combination thereof.

In various embodiments, the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 1 to 3. In various embodiments, the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 20 to 22. In various embodiments, the nucleic acid sequence encoding the phosphotransferase comprises a sequence having at least 70%, at least 80%, at least 90%, at least 95%, at least 96%, at least 97%, at least 98%, at least 99%, or 100% sequence identity to SEQ ID NO: 4 or 5. In various embodiments, the nucleic acid sequence encoding the phosphotransferase comprises a sequence having at least 70%, at least 80%, at least 90%, at least 95%, at least 96%, at least 97%, at least 98%, at least 99%, or 100% sequence identity to SEQ ID NO: 23 or 24. In various embodiments, the nucleic acid sequence encoding the methyltransferase comprises a sequence having at least 70%, at least 80%, at least 90%, at least 95%, at least 96%, at least 97%, at least 98%, or at least 99% sequence identity to SEQ ID NO: 6 or 7. In various embodiments, the nucleic acid sequence encoding the methyltransferase comprises a sequence having at least 70%, at least 80%, at least 90%, at least 95%, at least 96%, at least 97%, at least 98%, or at least 99%, or at least 100% sequence identity to SEQ ID NO: 25 or 26. In various embodiments, the nucleic acid sequence encoding the monooxygenase comprises a sequence having at least 70%, at least 80%, at least 90%, at least 95%, at least 96%, at least 97%, at least 98%, at least 99%, or 100% sequence identity to SEQ ID NO: 14. In various embodiments, the nucleic acid sequence encoding the monooxygenase comprises a sequence having at least 70%, at least 80%, at least 90%, at least 95%, at least 96%, at least 97%, at least 98%, at least 99%, or 100% sequence identity to SEQ ID NO: 33. In various embodiments, the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 2, 4, and 6. In various embodiments, the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 21, 23, and 25. In various embodiments, the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 2, 4, and 7. In various embodiments, the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 21, 23, and 26. In various embodiments, the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 24.

In various embodiments, the microbial cell is an Escherichia coli cell. The microbial cell comprises a tryptophanase A (tnaA) and tryptophanase B (tnaB) gene. In various embodiments, expression of the tnaA gene is reduced or prevented. The skilled person will understand that there are various methods known in the art (such as RNAi, CRISPR, or siRNA, for example) that can be used to reduce or prevent gene expression. In some embodiments, the tnaA gene is mutated to encode a non-functional protein. In some embodiments, the microbial cell is modified to not degrade L-arabinose.

In various embodiments, the one or more psilocybin production genes are in an operon operably linked to a promoter. In various embodiments, the operon is an araBAD operon that encodes the ribulokinase (AraB), L-arabinose isomerase (AraA), and L-ribulose-5-phosphate 4-epimerase (AraD) genes and is operably linked to an L-arabinose-inducible araBAD promoter (pBAD) which controls expression of the one or more psilocybin production genes through L-arabinose induction. In various embodiments, the microbial cell comprises an endogenous araA gene that is deleted or encodes a non-functional protein. The microbial cell comprises an arabinose transport (araE) gene. In various embodiments, the araE gene is operably linked to a constitutive promoter. In various embodiments, the araE gene comprises one or more ribosome binding sites (RBS) having relative activity that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold greater than the wild-type araE RBS. In various embodiments, the microbial cell comprises an L-arabinose binding (araF), L-arabinose import ATP-binding (araG), and L-arabinose transporter (araH) genes are deleted or encode non-functional proteins. In various embodiments, the L-arabinose binding (araF), L-arabinose import ATP-binding (araG), and L-arabinose transporter (araH) genes are endogenous genes.

In various embodiments, the microbial cell comprises a 5′-methylthioadenosine/S-adenosylhomocysteine nucleosidase (mtnN) gene having increased expression relative to a control. In various embodiments, the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 13. In various embodiments, the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 32. The skilled person will appreciate that some heterologous or exogenous genes may be provided in a plasmid vector or may be stably integrated within the bacterial genome.

In various embodiments, the microbial cell further comprises a hemin receptor gene. In various embodiments, the hemin receptor gene comprises a sequence having at least 70%, at least 80%, at least 90%, at least 95%, at least 96%, at least 97%, at least 98%, at least 99%, or 100% sequence identity to SEQ ID NO: 15. In various embodiments, the hemin receptor gene comprises a sequence having at least 70%, at least 80%, at least 90%, at least 95%, at least 96%, at least 97%, at least 98%, at least 99%, or 100% sequence identity to SEQ ID NO: 34.

In various embodiments, the heterologous nucleic acid comprises a NADPH-cytochrome P450 reductase (CPR) gene. In various embodiments, the CPR gene comprises a sequence having at least 70%, at least 80%, at least 90%, at least 95%, at least 96%, at least 97%, at least 98%, at least 99%, or 100% sequence identity to SEQ ID NO: 16, 17, or 18. In various embodiments, the CPR gene comprises a sequence having at least 70%, at least 80%, at least 90%, at least 95%, at least 96%, at least 97%, at least 98%, at least 99%, or 100% sequence identity to SEQ ID NO: 35, 36, or 37.

In various embodiments, expression of an endogenous soluble transhydrogenase (sthA) gene is reduced. In various embodiments, an endogenous soluble transhydrogenase (sthA) gene is mutated to encode a non-functional protein.

In some embodiments, the heterologous nucleic acid comprises one or more expression vectors. In some embodiments, the one or more expression vectors is or comprises one expression vector. In some embodiments, the one or more expression vectors is a high copy number vector or a low copy number vector. In some embodiments, the one or more psilocybin production genes in the operon are arranged in the following order: SEQ ID NO: 6, 4, and 2 or SEQ ID NO: 25, 23, and 21 or SEQ ID NO: 2, 4, and 6 or SEQ ID NO: 21, 23, and 25 or SEQ ID NO: 4, 2, and 7 or SEQ ID NO: 23, 21, and 26 or SEQ ID NO: 2, 6, and SEQ ID NO: 4 or 21, 25, and 23 or SEQ ID NO: 4, 2, and 6 or SEQ ID NO: 23, 21, and 25 or SEQ ID NO: 2, 4, and 7 or SEQ ID NO: 21, 23, and 26.

In some embodiments, the promoter is an inducible promoter. In some embodiments, the inducible promoter is an arabinose inducible promoter.

In various embodiments, the operon is an araBAD operon that encodes ribulokinase (AraB), L-arabinose isomerase (AraA), and L-ribulose-5-phosphate 4-epimerase (AraD) genes and is operably linked to an L-arabinose-inducible araBAD promoter (pBAD) which controls expression of the one or more psilocybin production genes through L-arabinose induction.

In some embodiments, the one or more psilocybin production genes are in an operon operably linked to a promoter which controls expression of the one or more psilocybin production genes. In some embodiments, the araA gene from the araBAD operon is deleted or encodes a non-functional protein.

In some embodiments, the inducible promoter is a glucose inducible promoter. In some embodiments, the inducible promoter is an isopropyl β-D-1-thiogalactopyranoside (IPTG) inducible promoter. In some embodiments, the inducible promoter is a tac1 promoter.

In another aspect, the disclosure pertains to a system for producing psilocybin comprising a bioreactor comprising a growth medium, and the recombinant microbial cell as defined herein.

In various embodiments, the growth medium is supplemented with one or more of the following supplements: L-tryptophan and anthranilate. In various embodiments, the microbial cell is cultured with one or more intermediates of psilocybin. In various embodiments, the intermediate is psilocin or 4-hydroxyindole. In various embodiments, psilocin is produced by chemical synthesis. In various embodiments, additional of L-arabinose induces the pBAD promoter to express genes operably linked to the promoter. In various embodiments, the L-arabinose concentration in the growth medium is 0.15%. In various embodiments, addition of glucose represses gene expression by the pBAD promoter. The skilled person will appreciate that some adjustments in the concentration of L-arabinose or glucose may be needed to induce or repress activity of the pBAD promoter.

In another aspect, the disclosure pertains to a system for producing psilocybin comprising a bioreactor comprising a growth medium, the recombinant microbial cell as defined herein, and arabinose, wherein arabinose induces expression of the one or more psilocybin producing genes. In some embodiments, a concentration of arabinose in the growth medium is between 0.015% to 0.15%. In some embodiments, a concentration of arabinose in the growth medium is about 0.015% or about 0.15%.

In some embodiments, addition of arabinose induces gene expression by the pBAD promoter of the one or more psilocybin producing genes. In some embodiments, addition of glucose represses gene expression of the one or more psilocybin producing genes by the pBAD promoter.

In some embodiments, the growth medium is supplemented with one or more of the following supplements: L-tryptophan and anthranilate.

In another aspect, the disclosure pertains to a system for producing psilocybin comprising a bioreactor comprising a growth medium, the recombinant microbial cell as defined herein, glucose, and IPTG, wherein IPTG induces expression of the one or more psilocybin producing genes. In some embodiments, a concentration of glucose in the growth medium is about 2%. In some embodiments, a concentration of IPTG is about 0.001 mM, 0.01 mM, or 0.1 mM. In some embodiments, IPTG induced gene expression of the one or more psilocybin producing genes is driven by the tac1 promoter.

In another aspect, the disclosure pertains to a method of producing psilocybin comprising providing the recombinant microbial cell as defined herein, and culturing the recombinant microbial cell in a growth medium. In another aspect, the disclosure pertains to a method of producing psilocybin comprising culturing the recombinant microbial cell as defined herein in a growth medium. In some embodiments, the method comprises isolating the psilocybin synthesized by the microbial cell from the growth medium. In some embodiments, the method comprises supplementing the growth medium with one or more of the following supplements: L-tryptophan and anthranilate. In some embodiments, the microbial cell is engineered to produce an increased level of tryptophan. In some embodiments, the microbial cell is cultured with one or more intermediates of psilocybin. In some embodiments, the one or more intermediates is psilocin. In some embodiments, the one or more intermediates is 4-hydroxy tryptamine. In some embodiments, the one or more intermediates are chemically synthesized.

In another aspect, the disclosure pertains to a method for producing psilocybin comprising culturing the recombinant microbial cell as defined herein in a growth medium and adding arabinose to the growth medium to induce expression of the one or more psilocybin producing genes. In some embodiments, adding arabinose induces the pBAD promoter to express the one or more psilocybin producing genes. In some embodiments, adding glucose to the growth medium represses the pBAD promoter.

In another aspect, the disclosure pertains to a method for producing psilocybin comprising culturing the recombinant microbial cell as defined herein in a growth medium comprising glucose and adding IPTG to induce expression of the one or more psilocybin producing genes. In some embodiments, adding IPTG induces the tac1 promoter to express the one or more psilocybin producing genes.

In various embodiments, the step of culturing the recombinant microbial cell in the growth medium to produce psilocybin comprises inducing the pBAD promoter to express genes operably linked to the promoter by adding L-arabinose. In various embodiments, the step of culturing the recombinant microbial cell in the growth medium comprises inducing the pBAD promoter to express genes operably linked to the promoter by adding L-arabinose. In various embodiments, the step of culturing the recombinant microbial cell in the growth medium comprises repressing the pBAD promoter by adding glucose.

In another aspect, the disclosure pertains to a nucleic acid molecule comprising a sequence having at least 70%, at least 80%, at least 90%, at least 95%, at least 96%, at least 97%, at least 98%, at least 99%, or 100% sequence identity to any one of SEQ ID NOs: 1 to 7, 13 to 18, 20 to 26, and 32 to 37, or a combination thereof.

In another aspect, the disclosure pertains to an expression vector for producing psilocybin in a microbial cell, the expression vector comprising the nucleic acid molecule as defined herein.

In another aspect, the disclosure pertains to psilocybin produced from the method as defined herein for treating a mental health condition. In some embodiments, the mental health condition is suicidality, depression, obsessive-compulsive disorder, anxiety, alcohol dependence, tobacco dependence, cocaine-related disorders, cluster headache, and post-traumatic stress disorder.

In another aspect, the disclosure pertains to a pharmaceutical composition comprising psilocybin produced from the method as defined herein and a pharmaceutically acceptable carrier for treating a mental health condition. In some embodiments, the mental health condition is suicidality, depression, obsessive-compulsive disorder, anxiety, alcohol dependence, tobacco dependence, cocaine-related disorders, cluster headache, and post-traumatic stress disorder.

In another aspect, the disclosure pertains to use of psilocybin produced from the method as defined herein for treating a mental health condition.

In another aspect, the disclosure pertains to use of psilocybin produced from the method as defined herein in preparation of a medicament for treating a mental health condition.

In various embodiments, the mental health condition is suicidality, depression, obsessive-compulsive disorder, anxiety, alcohol dependence, tobacco dependence, cocaine-related disorders, cluster headache, and post-traumatic stress disorder.

In another aspect, the disclosure pertains to use of a pharmaceutical composition comprising psilocybin produced from the method as defined herein and a pharmaceutically acceptable carrier for treating a mental health condition.

In another aspect, the disclosure pertains to use of a pharmaceutical composition comprising psilocybin produced from the method as defined herein and a pharmaceutically acceptable carrier in preparation of a medicament for treating a mental health condition.

Various aspects of the disclosure pertain to a recombinant microbial cell comprising a biosynthetic pathway for producing dimethyltryptamine (DMT), or intermediates thereof, the microbial cell comprising a heterologous nucleic acid encoding one or more DMT production genes. In some embodiments, the heterologous nucleic acid comprises one or more expression vectors. In some embodiments, the one or more DMT production genes is a tryptophan decarboxylase, an indolethylamine N-methyltransferase (INMT), or a combination thereof. In some embodiments, the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 1 to 3.

In some embodiments, the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 20 to 22. In some embodiments, the INMT comprises a sequence having at least 70%, at least 80%, at least 90%, at least 95%, at least 96%, at least 97%, at least 98%, at least 99%, or 100% sequence identity to any one of SEQ ID NOs: 8 to 12. In some embodiments, the INMT comprises a sequence having at least 70%, at least 80%, at least 90%, at least 95%, at least 96%, at least 97%, at least 98%, at least 99%, or 100% sequence identity to any one of SEQ ID NOs: 27 to 31.

In various embodiments, wherein the microbial cell comprises a 5′-methylthioadenosine/S-adenosylhomocysteine nucleosidase (mtnN) gene having increased expression relative to a control. In various embodiments, the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 13. In various embodiments, the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 32. In various embodiments, the microbial cell is an Escherichia coli cell. In various embodiments, the microbial cell comprises a tryptophanase A (tnaA) and tryptophanase B (tnaB) gene. In various embodiments, expression of the tnaA gene is reduced or prevented. In various embodiments, the microbial cell is modified to not degrade L-arabinose.

In various embodiments, the microbial cell comprises an araA gene that is deleted or encodes a non-functional protein. In various embodiments, the microbial cell comprises an araE gene.

In some embodiments, the araE gene is operably linked to a constitutive promoter. In some embodiments, the araE gene comprises one or more ribosome binding sites (RBS) having relative activity that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold greater than the native araE RBS. In some embodiments, araF, araG, and araH genes are deleted or encode non-functional proteins.

In some embodiments, the heterologous nucleic acid comprises one or more expression vectors. In some embodiments, the one or more expression vectors is or comprises one expression vector. In some embodiments, the one or more expression vectors is a high copy number vector or a low copy number vector. In some embodiments, the one or more DMT production genes are in an operon operably linked to a promoter which controls expression of the one or more DMT production genes. In some embodiments, the promoter is an inducible promoter. In some embodiments, the inducible promoter is an arabinose inducible promoter. In some embodiments, the operon is an araBAD operon operably linked to an L-arabinose-inducible araBAD promoter (pBAD) which controls expression of the one or more DMT production genes through L-arabinose induction. In some embodiments, the araA gene from the araBAD operon is deleted or encodes a non-functional protein.

In another aspect, the disclosure pertains to a system for producing dimethyltryptamine (DMT) comprising a bioreactor comprising a growth medium, and the recombinant microbial cell as defined herein. In various embodiments, the growth medium is supplemented with one or more of the following supplements: L-tryptophan and anthranilate. In various embodiments, addition of L-arabinose induces the pBAD promoter to express genes operably linked to the promoter. In various embodiments, the L-arabinose concentration is 0.15%. In various embodiments, addition of glucose represses gene expression by the pBAD promoter.

In another aspect, the disclosure pertains to a method of producing dimethyltryptamine (DMT) comprising providing the recombinant microbial cell as defined herein, and cultivating the recombinant microbial cell in a growth medium to produce DMT. In another aspect, the disclosure pertains to a method of producing dimethyltryptamine (DMT) comprising culturing the recombinant microbial cell as defined herein in a growth medium to produce DMT. In various embodiments, the method comprises isolating the psilocybin synthesized by the recombinant microbial cell from the growth medium. In various embodiments, wherein the method comprises supplementing the growth medium with one or more of the following supplements: L-tryptophan and anthranilate. In various embodiments, the microbial cell produces an increased level of tryptophan.

In various embodiments, the step of culturing the recombinant microbial cell in the growth medium to produce DMT comprises inducing the pBAD promoter to express genes operably linked to the promoter by adding L-arabinose. In various embodiments, the step of culturing the recombinant microbial cell in the growth medium to produce DMT comprises repressing the pBAD promoter by adding glucose.

In various embodiments, the method comprises adding arabinose to the growth medium to induce the pBAD promoter to express the psilocybin producing genes operably linked to the promoter. In various embodiments, method comprises adding glucose to the growth medium to repress expression of the psilocybin producing genes operably linked to the pBAD promoter.

In another aspect, the disclosure pertains to a nucleic acid molecule comprising a sequence having at least 70%, at least 80%, at least 90%, at least 95%, at least 96%, at least 97%, at least 98%, at least 99%, or 100% sequence identity to any one of SEQ ID NOs: 1 to 3, 8 to 13, 20 to 22, and 27 to 32, or a combination thereof.

In another aspect, the disclosure pertains to an expression vector for producing psilocybin in a microbial cell, the expression vector comprising the nucleic acid molecule as defined herein.

In another aspect, the disclosure pertains to dimethyltryptamine (DMT) produced from the method as defined herein for treating a mental health condition. In various embodiments, the mental health condition is suicidality, depression, obsessive-compulsive disorder, anxiety, alcohol dependence, tobacco dependence, cocaine-related disorders, cluster headache, and post-traumatic stress disorder.

In another aspect, the disclosure pertains to a pharmaceutical composition comprising dimethyltryptamine (DMT) produced from the method as defined herein and a pharmaceutically acceptable carrier for treating a mental health condition. In various embodiments, the mental health condition is suicidality, depression, obsessive-compulsive disorder, anxiety, alcohol dependence, tobacco dependence, cocaine-related disorders, cluster headache, and post-traumatic stress disorder.

In another aspect, the disclosure pertains to use of dimethyltryptamine (DMT) produced from the method as defined herein for treating a mental health condition.

In another aspect, the disclosure pertains to use of dimethyltryptamine (DMT) produced from the method as defined herein in preparation of a medicament for treating a mental health condition. In some embodiments, the mental health condition is suicidality, depression, obsessive-compulsive disorder, anxiety, alcohol dependence, tobacco dependence, cocaine-related disorders, cluster headache, and post-traumatic stress disorder.

In another aspect, the disclosure pertains to use of a pharmaceutical composition comprising dimethyltryptamine (DMT) produced from the method as defined herein and a pharmaceutically acceptable carrier for treating a mental health condition.

In another aspect, the disclosure pertains to use of a pharmaceutical composition comprising dimethyltryptamine (DMT) produced from the method as defined herein and a pharmaceutically acceptable carrier in preparation of a medicament for treating a mental health condition.

While specific embodiments have been described and illustrated, such embodiments should be considered illustrative of the subject matter described herein and not as limiting the claims as construed in accordance with the relevant jurisprudence.

All applications and publications referred to herein are incorporated by reference in their entirety.

Embodiments in which an exclusive property or privilege is claimed are defined as follows:

1. A recombinant microbial cell comprising a biosynthetic pathway for producing psilocybin, or intermediates thereof, the microbial cell comprising a heterologous nucleic acid encoding one or more psilocybin production genes.

2. The microbial cell of claim 1, wherein the one or more psilocybin production genes is a tryptophan decarboxylase, a phosphotransferase, a methyltransferase, a monooxygenase, or a combination thereof.

3. The microbial cell of claim 2, wherein the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 1 to 3.

4. The microbial cell of claim 2, wherein the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 20 to 22.

5. The microbial cell of any one of claims 2 to 4, wherein the nucleic acid sequence encoding the phosphotransferase comprises a sequence having at least 70%, at least 80%, at least 90%, at least 95%, at least 96%, at least 97%, at least 98%, at least 99%, or 100% sequence identity to SEQ ID NO: 4 or 5.

6. The microbial cell of any one of claims 2 to 4, wherein the nucleic acid sequence encoding the phosphotransferase comprises a sequence having at least 70%, at least 80%, at least 90%, at least 95%, at least 96%, at least 97%, at least 98%, at least 99%, or 100% sequence identity to SEQ ID NO: 23 or 24.

7. The microbial cell of any one of claims 2 to 6, wherein the nucleic acid sequence encoding the methyltransferase comprises a sequence having at least 70%, at least 80%, at least 90%, at least 95%, at least 96%, at least 97%, at least 98%, or at least 99%, or at least 100% sequence identity to SEQ ID NO: 6 or 7.

8. The microbial cell of any one of claims 2 to 6, wherein the nucleic acid sequence encoding the methyltransferase comprises a sequence having at least 70%, at least 80%, at least 90%, at least 95%, at least 96%, at least 97%, at least 98%, or at least 99%, or at least 100% sequence identity to SEQ ID NO: 25 or 26.

9. The microbial cell of any one of claims 2 to 8, wherein the nucleic acid sequence encoding the monooxygenase comprises a sequence having at least 70%, at least 80%, at least 90%, at least 95%, at least 96%, at least 97%, at least 98%, at least 99%, or 100% sequence identity to SEQ ID NO: 14.

10. The microbial cell of any one of claims 2 to 8, wherein the nucleic acid sequence encoding the monooxygenase comprises a sequence having at least 70%, at least 80%, at least 90%, at least 95%, at least 96%, at least 97%, at least 98%, at least 99%, or 100% sequence identity to SEQ ID NO: 33.

11. The microbial cell of claim 2, wherein the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 2, 4, and 6.

12. The microbial cell of claim 2, wherein the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 21, 23, and 25.

13. The microbial cell of claim 2, wherein the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 2, 4, and 7.

14. The microbial cell of claim 2, wherein the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 21, 23, and 26.

15. The microbial cell of claim 2, wherein the nucleic acid encoding the one or more psilocybin production genes comprises a sequence having at least 70%, at least 80%, at least 90%, at least 95%, at least 96%, at least 97%, at least 98%, at least 99%, or 100% sequence identity to SEQ ID NO: 24.

16. The microbial cell of any one of claims 1 to 15, wherein the microbial cell is an Escherichia coli cell.

17. The microbial cell of any one of claims 1 to 16, wherein the microbial cell comprises a tryptophanase A (tnaA) and tryptophanase B (tnaB) gene.

18. The microbial cell of claim 17, wherein expression of the tnaA gene is reduced or prevented, or the tnaA gene is mutated to encode a non-functional protein.

19. The microbial cell of any one of claims 1 to 18, wherein the microbial cell is modified to not degrade L-arabinose.

20. The microbial cell of any one of claims 1 to 19, wherein the microbial cell comprises an endogenous araA gene that is deleted or encodes a non-functional protein.

21. The microbial cell of any one of claims 1 to 20, wherein the microbial cell comprises an arabinose transport (araE) gene.

22. The microbial cell of claim 21, wherein the araE gene is operably linked to a constitutive promoter.

23. The microbial cell of claim 21 or 22, wherein the araE gene comprises one or more ribosome binding sites (RBS) having relative activity that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold greater than the wild-type araE RBS.

24. The microbial cell of claim 21, 22, or 23, wherein the microbial cell comprises an L-arabinose binding protein (araF), L-arabinose import ATP-binding (araG), and L-arabinose transporter (araH) genes that are deleted or encode non-functional proteins.

25. The microbial cell of any one of claims 1 to 24, wherein the microbial cell comprises a 5′-methylthioadenosine/S-adenosylhomocysteine nucleosidase (mtnN) gene having increased expression relative to a control.

26. The microbial cell of any one of claims 1 to 24, wherein the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 13.

27. The microbial cell of any one of claims 1 to 24, wherein the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 32.

28. The microbial cell of any one of claims 1 to 27, wherein the microbial cell further comprises a hemin receptor gene.

29. The microbial cell of claim 28, wherein the hemin receptor gene comprises a sequence having at least 70%, at least 80%, at least 90%, at least 95%, at least 96%, at least 97%, at least 98%, at least 99%, or 100% sequence identity to SEQ ID NO: 15.

30. The microbial cell of claim 28, wherein the hemin receptor gene comprises a sequence having at least 70%, at least 80%, at least 90%, at least 95%, at least 96%, at least 97%, at least 98%, at least 99%, or 100% sequence identity to SEQ ID NO: 34.

31. The microbial cell of any one of claims 1 to 30, wherein the heterologous nucleic acid comprises a NADPH-cytochrome P450 reductase (CPR) gene.

32. The microbial cell of claim 31, wherein the CPR gene comprises a sequence having at least 70%, at least 80%, at least 90%, at least 95%, at least 96%, at least 97%, at least 98%, at least 99%, or 100% sequence identity to SEQ ID NO: 16, 17, or 18.

33. The microbial cell of claim 31, wherein the CPR gene comprises a sequence having at least 70%, at least 80%, at least 90%, at least 95%, at least 96%, at least 97%, at least 98%, at least 99%, or 100% sequence identity to SEQ ID NO: 35, 36, or 37.

34. The microbial cell of any one of claims 1 to 33, wherein expression of an endogenous soluble transhydrogenase (sthA) gene is reduced.

35. The microbial cell of any one of claims 1 to 33, wherein an endogenous soluble transhydrogenase (sthA) gene is mutated to encode a non-functional protein.

36. The microbial cell of any one of claims 1 to 35, wherein the heterologous nucleic acid comprises one or more expression vectors.

37. The microbial cell of claim 36, wherein the one or more expression vectors is or comprises one expression vector.

38. The microbial cell of claim 36 or 37, wherein the one or more expression vectors is a high copy number vector.

39. The microbial cell of claim 36 or 37, wherein the one or more expression vectors is a low copy number vector.

40. The microbial cell of any one of claims 1 to 39, wherein the one or more psilocybin production genes are in an operon operably linked to a promoter which controls expression of the one or more psilocybin production genes.

41. The microbial cell of claim 40, wherein the one or more psilocybin production genes in the operon are arranged in the following order: SEQ ID NO: 6, 4, and 2.

42. The microbial cell of claim 40, wherein the one or more psilocybin production genes in the operon are arranged in the following order: SEQ ID NO: 25, 23, and 21.

43. The microbial cell of claim 40, wherein the one or more psilocybin production genes in the operon are arranged in the following order: SEQ ID NO: 2, 4, and 6.

44. The microbial cell of claim 40, wherein the one or more psilocybin production genes in the operon are arranged in the following order: SEQ ID NO: 21, 23, and 25.

45. The microbial cell of claim 40, wherein the one or more psilocybin production genes in the operon are arranged in the following order: SEQ ID NO: 4, 2, and 7.

46. The microbial cell of claim 40, wherein the one or more psilocybin production genes in the operon are arranged in the following order: SEQ ID NO: 23, 21, and 26.

47. The microbial cell of claim 40, wherein the one or more psilocybin production genes in the operon are arranged in the following order: SEQ ID NO: 2, 6, and 4.

48. The microbial cell of claim 40, wherein the one or more psilocybin production genes in the operon are arranged in the following order: SEQ ID NO: 21, 25, and 23.

49. The microbial cell of claim 40, wherein the one or more psilocybin production genes in the operon are arranged in the following order: SEQ ID NO: 4, 2, and 6.

50. The microbial cell of claim 40, wherein the one or more psilocybin production genes in the operon are arranged in the following order: SEQ ID NO: 23, 21, and 25.

51. The microbial cell of claim 40, wherein the one or more psilocybin production genes in the operon are arranged in the following order: SEQ ID NO: 2, 4, and 7.

52. The microbial cell of claim 40, wherein the one or more psilocybin production genes in the operon are arranged in the following order: SEQ ID NO: 21, 23, and 26.

53. The microbial cell of any one of claims 40 to 52, wherein the promoter is an inducible promoter.

54. The microbial cell of claim 53, wherein the inducible promoter is an arabinose inducible promoter.

55. The microbial cell of claim 53 or 54, wherein the operon is an araBAD operon that encodes ribulokinase (AraB), L-arabinose isomerase (AraA), and L-ribulose-5-phosphate 4-epimerase (AraD) genes and is operably linked to an L-arabinose-inducible araBAD promoter (pBAD) which controls expression of the one or more psilocybin production genes through L-arabinose induction.

56. The microbial cell of claim 55, wherein the araA gene from the araBAD operon is deleted or encodes a non-functional protein.

57. The microbial cell of claim 53, wherein the inducible promoter is a glucose inducible promoter.

58. The microbial cell of claim 57, wherein the inducible promoter is an isopropyl 3-D-1-thiogalactopyranoside (IPTG) inducible promoter.

59. The microbial cell of claim 57, wherein the inducible promoter is a tac1 promoter.

60. A system for producing psilocybin comprising a bioreactor comprising a growth medium, and the recombinant microbial cell as defined in any one of claims 1 to 59.

61. The system of claim 60, wherein the growth medium is supplemented with one or more of the following supplements: L-tryptophan and anthranilate.

62. The system of claim 60 or 61, wherein the microbial cell is cultured with one or more intermediates of psilocybin.

63. The system of claim 62, wherein the intermediate is psilocin or 4-hydroxyindole.

64. The system of claim 63, wherein psilocin is produced by chemical synthesis.

65. A system for producing psilocybin comprising a bioreactor comprising a growth medium, the recombinant microbial cell as defined in any one of claims 1 to 56, and arabinose, wherein arabinose induces expression of the one or more psilocybin producing genes.

66. The system of claim 65, wherein a concentration of arabinose in the growth medium is between 0.015% to 0.15%.

67. The system of claim 65, wherein a concentration of arabinose in the growth medium is about 0.015%.

68. The system of claim 65, wherein a concentration of arabinose in the growth medium is about 0.15%.

69. The system of any one of claims 65 to 68, wherein addition of arabinose induces gene expression by the pBAD promoter of the one or more psilocybin producing genes.

70. The system of any one of claims 65 to 69, wherein addition of glucose represses gene expression of the one or more psilocybin producing genes by the pBAD promoter.

71. The system of any one of claims 65 to 70, wherein the growth medium is supplemented with one or more of the following supplements: L-tryptophan and anthranilate.

72. The system of any one of claims 65 to 71, wherein the microbial cell is cultured with one or more intermediates of psilocybin or 4-hydroxyindole.

73. The system of claim 72, wherein the intermediate is psilocin.

74. The system of claim 73, wherein psilocin is produced by chemical synthesis.

75. A system for producing psilocybin comprising a bioreactor comprising a growth medium, the recombinant microbial cell as defined in any one of claims 1 to 53 and 57 to 59, glucose, and IPTG, wherein IPTG induces expression of the one or more psilocybin producing genes.

76. The system of claim 75, wherein a concentration of glucose in the growth medium is about 2%.

77. The system of claim 75 or 76, wherein a concentration of IPTG is about 0.001 mM, 0.01 mM, or 0.1 mM.

78. The system of any one of claims 75 to 77, wherein IPTG induced gene expression of the one or more psilocybin producing genes is driven by the tac1 promoter.

79. The system of any one of claims 75 to 78, wherein the growth medium is supplemented with one or more of the following supplements: L-tryptophan and anthranilate.

80. The system of any one of claims 75 to 79, wherein the microbial cell is cultured with one or more intermediates of psilocybin.

81. The system of claim 80, wherein the intermediate is psilocin or 4-hydroxyindole.

82. A method of producing psilocybin comprising culturing the recombinant microbial cell as defined in any one of claims 1 to 59 in a growth medium.

83. The method of claim 82, wherein the method comprises isolating the psilocybin synthesized by the microbial cell from the growth medium.

84. The method of claim 82 or 83, wherein the method comprises supplementing the growth medium with one or more of the following supplements: L-tryptophan and anthranilate.

85. The method of claim 82, 83, or 84, wherein the microbial cell is engineered to produce an increased level of tryptophan.

86. The method of any one of claims 82 to 85, wherein the microbial cell is cultured with one or more intermediates of psilocybin.

87. The method of claim 86, wherein the one or more intermediates is psilocin.

88. The method of claim 86 or 87, wherein the one or more intermediates is 4-hydroxy tryptamine.

89. The method of claim 86, 87, or 88, wherein the one or more intermediates are chemically synthesized.

90. A method for producing psilocybin comprising culturing the recombinant microbial cell as defined in any one of claims 1 to 56 in a growth medium and adding arabinose to the growth medium to induce expression of the one or more psilocybin producing genes.

91. The method of claim 90, wherein adding arabinose induces the pBAD promoter to express the one or more psilocybin producing genes.

92. The method of claim 90 or 91, wherein adding glucose to the growth medium represses the pBAD promoter.

93. A method for producing psilocybin comprising culturing the recombinant microbial cell as defined in any one of claims 1 to 53 and 57 to 59 in a growth medium comprising glucose and adding IPTG to induce expression of the one or more psilocybin producing genes.

94. The method of claim 93, wherein adding IPTG induces the tac1 promoter to express the one or more psilocybin producing genes.

95. A nucleic acid molecule comprising a sequence having at least 70%, at least 80%, at least 90%, at least 95%, at least 96%, at least 97%, at least 98%, at least 99%, or 100% sequence identity to any one of SEQ ID NOs: 1 to 7, 13 to 18, 20 to 26, and 32 to 37, or a combination thereof.

96. An expression vector for producing psilocybin in a microbial cell, the expression vector comprising the nucleic acid molecule as defined in claim 95.

97. Psilocybin produced from the method as defined in any one of claims 82 to 94 for treating a mental health condition.

98. The psilocybin compound of claim 97, wherein the mental health condition is suicidality, depression, obsessive-compulsive disorder, anxiety, alcohol dependence, tobacco dependence, cocaine-related disorders, cluster headache, and post-traumatic stress disorder.

99. A pharmaceutical composition comprising psilocybin produced from the method as defined in any one of claims 82 to 94 and a pharmaceutically acceptable carrier for treating a mental health condition.

100. The pharmaceutical composition of claim 99, wherein the mental health condition is suicidality, depression, obsessive-compulsive disorder, anxiety, alcohol dependence, tobacco dependence, cocaine-related disorders, cluster headache, and post-traumatic stress disorder.

101. Use of psilocybin produced from the method as defined in any one of claims 82 to 94 for treating a mental health condition.

102. Use of psilocybin produced from the method as defined in any one of claims 82 to 94 in preparation of a medicament for treating a mental health condition.

103. The use of claim 101 or 102, wherein the mental health condition is suicidality, depression, obsessive-compulsive disorder, anxiety, alcohol dependence, tobacco dependence, cocaine-related disorders, cluster headache, and post-traumatic stress disorder.

104. Use of a pharmaceutical composition comprising psilocybin produced from the method as defined in any one of claims 82 to 94 and a pharmaceutically acceptable carrier for treating a mental health condition.

105. Use of a pharmaceutical composition comprising psilocybin produced from the method as defined in any one of claims 82 to 94 and a pharmaceutically acceptable carrier in preparation of a medicament for treating a mental health condition.

106. A recombinant microbial cell comprising a biosynthetic pathway for producing dimethyltryptamine (DMT), or intermediates thereof, the microbial cell comprising a heterologous nucleic acid encoding one or more DMT production genes.

107. The microbial cell of claim 106, wherein the one or more DMT production genes is a tryptophan decarboxylase, an indolethylamine N-methyltransferase (INMT), or a combination thereof.

108. The microbial cell of claim 107, wherein the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 1 to 3.

109. The microbial cell of claim 107, wherein the nucleic acid sequence encoding the tryptophan decarboxylase comprises a sequence having at least 70%, at least 80%, at least 90%, at least 95%, at least 96%, at least 97%, at least 98%, at least 99%, or 100% sequence identity to any one of SEQ ID NOs: 20 to 22.

110. The microbial cell of any one of claims 107 to 109, wherein the INMT comprises a sequence having at least 70%, at least 80%, at least 90%, at least 95%, at least 96%, at least 97%, at least 98%, at least 99%, or 100% sequence identity to any one of SEQ ID NOs: 8 to 12.

111. The microbial cell of any one of claims 107 to 109, wherein the INMT comprises a sequence having at least 70%, at least 80%, at least 90%, at least 95%, at least 96%, at least 97%, at least 98%, at least 99%, or 100% sequence identity to any one of SEQ ID NOs: 27 to 31.

112. The microbial cell of any one of claims 106 to 111, wherein the microbial cell comprises a 5′-methylthioadenosine/S-adenosylhomocysteine nucleosidase (mtnN) gene having increased expression relative to a control.

113. The microbial cell of any one of claims 106 to 111, wherein the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 13.

114. The microbial cell of any one of claims 106 to 111, wherein the heterologous nucleic acid comprises an mtnN gene comprising a sequence having at least 70%, at least 80%, at least 90%, at least 95%, at least 96%, at least 97%, at least 98%, at least 99%, or 100% sequence identity to SEQ ID NO: 32.

115. The microbial cell of any one of claims 106 to 114, wherein the microbial cell is an Escherichia coli cell.

116. The microbial cell of any one of claims 106 to 115, wherein the microbial cell comprises a tryptophanase A (tnaA) and tryptophanase B (tnaB) gene.

117. The microbial cell of claim 116, wherein expression of the tnaA gene is reduced or prevented.

118. The microbial cell of claim 116, wherein the tnaA gene is mutated to encode a non-functional protein.

119. The microbial cell of any one of claims 106 to 118, wherein the microbial cell is modified to not degrade L-arabinose.

120. The microbial cell of any one of claims 106 to 119, wherein the microbial cell comprises an araA gene that is deleted or encodes a non-functional protein.

121. The microbial cell of any one of claims 106 to 120, wherein the microbial cell comprises an araE gene.

122. The microbial cell of claim 121, wherein the araE gene is operably linked to a constitutive promoter.

123. The microbial cell of claim 121 or 122, wherein the araE gene comprises one or more ribosome binding sites (RBS) having relative activity that is at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 35-fold, at least 40-fold, at least 45-fold, or at least 50-fold greater than the native araE RBS.

124. The microbial cell of claim 121, 122, or 123, wherein araF, araG, and araH genes are deleted or encode non-functional proteins.

125. The microbial cell of any one of claims 106 to 124, wherein the heterologous nucleic acid comprises one or more expression vectors.

126. The microbial cell of claim 125, wherein the one or more expression vectors is or comprises one expression vector.

127. The microbial cell of claim 125 or 126, wherein the one or more expression vectors is a high copy number vector.

128. The microbial cell of claim 125 or 126, wherein the one or more expression vectors is a low copy number vector.

129. The microbial cell of any one of claims 106 to 128, wherein the one or more DMT production genes are in an operon operably linked to a promoter which controls expression of the one or more DMT production genes.

130. The microbial cell of claim 129, wherein the promoter is an inducible promoter.

131. The microbial cell of claim 130, wherein the inducible promoter is an arabinose inducible promoter.

132. The microbial cell of claim 131, wherein the operon is an araBAD operon operably linked to an L-arabinose-inducible araBAD promoter (pBAD) which controls expression of the one or more DMT production genes through L-arabinose induction.

133. The microbial cell of claim 132, wherein the araA gene from the araBAD operon is deleted or encodes a non-functional protein.

134. A system for producing dimethyltryptamine (DMT) comprising a bioreactor comprising a growth medium, and the recombinant microbial cell as defined in any one of claims 106 to 133.

135. The system of claim 134, wherein the growth medium is supplemented with one or more of the following supplements: L-tryptophan and anthranilate.

136. The system of claim 134 or 135, wherein addition of L-arabinose induces the pBAD promoter to express genes operably linked to the promoter.

137. The system of claim 136, wherein the L-arabinose concentration is 0.15%.

138. The system of any one of claims 134 to 137, wherein addition of glucose represses gene expression by the pBAD promoter.

139. A method of producing dimethyltryptamine (DMT) comprising culturing the recombinant microbial cell as defined in any one of claims 106 to 133 in a growth medium to produce DMT.

140. The method of claim 139, wherein the method comprises isolating the DMT synthesized by the recombinant microbial cell from the growth medium.

141. The method of claim 139 or 140, wherein the method comprises supplementing the growth medium with one or more of the following supplements: L-tryptophan and anthranilate.

142. The method of any one of claims 139 to 141, wherein the microbial cell produces an increased level of tryptophan.

143. The method of any one of claims 139 to 142, wherein the method comprises adding arabinose to the growth medium to induce the pBAD promoter to express the DMT producing genes operably linked to the promoter.

144. The method of any one of claims 139 to 142, wherein the method comprises adding glucose to the growth medium to repress expression of the DMT producing genes operably linked to the pBAD promoter.

145. A nucleic acid molecule comprising a sequence having at least 70%, at least 80%, at least 90%, at least 95%, at least 96%, at least 97%, at least 98%, at least 99%, or 100% sequence identity to any one of SEQ ID NOs: 1 to 3, 8 to 13, 20 to 22, and 27 to 32, or a combination thereof.

146. An expression vector for producing dimethyltryptamine (DMT) in a microbial cell, the expression vector comprising the nucleic acid molecule as defined in claim 145.

147. Dimethyltryptamine (DMT) produced from the method as defined in any one of claims 139 to 144 for treating a mental health condition.

148. The dimethyltryptamine compound of claim 147, wherein the mental health condition is suicidality, depression, obsessive-compulsive disorder, anxiety, alcohol dependence, tobacco dependence, cocaine-related disorders, cluster headache, and post-traumatic stress disorder.

149. A pharmaceutical composition comprising dimethyltryptamine (DMT) produced from the method as defined in any one of claims 139 to 144 and a pharmaceutically acceptable carrier for treating a mental health condition.

150. The pharmaceutical composition of claim 149, wherein the mental health condition is suicidality, depression, obsessive-compulsive disorder, anxiety, alcohol dependence, tobacco dependence, cocaine-related disorders, cluster headache, and post-traumatic stress disorder.

151. Use of dimethyltryptamine (DMT) produced from the method as defined in any one of claims 139 to 144 for treating a mental health condition.

152. Use of dimethyltryptamine (DMT) produced from the method as defined in any one of claims 139 to 144 in preparation of a medicament for treating a mental health condition.

153. The use of claim 151 or 152, wherein the mental health condition is suicidality, depression, obsessive-compulsive disorder, anxiety, alcohol dependence, tobacco dependence, cocaine-related disorders, cluster headache, and post-traumatic stress disorder.

154. Use of a pharmaceutical composition comprising dimethyltryptamine (DMT) produced from the method as defined in any one of claims 139 to 144 and a pharmaceutically acceptable carrier for treating a mental health condition.

155. Use of a pharmaceutical composition comprising dimethyltryptamine (DMT) produced from the method as defined in any one of claims 135 to 140 and a pharmaceutically acceptable carrier in preparation of a medicament for treating a mental health condition.
